(19) United States
(12) Patent Application Publication
(10) Pub. No.: US 2015/0056707 A1
(43) Pub. Date: Feb. 26, 2015

(54) ENGINEERING SALT TOLERANCE IN PHOTOSYNTHETIC MICROORGANISMS

(71) Applicant: SAPPHIRE ENERGY, INC., SAN DIEGO, CA (US)

(72) Inventors: MICHAEL MENDEZ, SAN DIEGO, CA (US); SU-CHIUNG FANG, SINSHIH TOWNSHIP (TW); STEPHANE RICHARD, SAN DIEGO, CA (US)

(73) Assignee: SAPPHIRE ENERGY, INC., SAN DIEGO, CA (US)

(21) Appl. No.: 14/532,681

(22) Filed: Nov. 4, 2014

Related U.S. Application Data
(63) Continuation of application No. 13/255,878, filed on Dec. 5, 2011.
(60) Provisional application No. 61/159,384, filed on Mar. 11, 2009.

(51) Int. Cl. C12N 15/82; C12N 9/88; C12N 15/74; C12N 9/14

(52) U.S. Cl. CPC C12N 15/8273; C12N 9/14; C12N 9/88; C12N 15/74; C12Y 306/01003; C12Y 402/03016; C12Y 402/0302
USPC 435/471; 435/257.2

(57) ABSTRACT

Provided herein are compositions and methods for engineering salt tolerance and producing products by photosynthetic organisms. The photosynthetic organisms can be genetically modified to be salt tolerant as compared to an unmodified organism and to produce useful products. The methods and compositions of the disclosure are useful in many therapeutic and industrial applications.

### CROSS REFERENCE TO RELATED APPLICATION

This application is a continuation of U.S. patent application Ser. No. 13/255,878 filed Dec. 5, 2011 which is the national phase of International Patent Application Number PCT/US2010/027039, filed Mar. 11, 2010, which claims the benefit of U.S. Provisional Application No. 61/159,384, filed Mar. 11, 2009, each of which is incorporated by reference in its entirety for all purposes.

### INCORPORATION BY REFERENCE

All publications, patents, patent applications, public databases, public database entries, and other references cited in this application are herein incorporated by reference in their entirety as if each individual publication, patent, patent application, public database, public database entry, or other reference was specifically and individually indicated to be incorporated by reference.

### BACKGROUND

Large scale cultivation of photosynthetic organisms requires a relatively controlled environment with a large input of light energy. Most commercial production techniques use large open ponds, taking advantage of natural sunlight. These systems have a relatively low surface area to volume ratio with corresponding low cell densities. It is very difficult to prevent contaminating organisms from invading an open pond. The potential for contamination restricts the usefulness of open ponds to organisms that thrive in conditions not suitable for the growth of most contaminating organisms.

The ability of organisms to grow and metabolize under high salt growth conditions is desirable for the cultivation of such organisms, in order to minimize microbial contaminations. Salinity has been shown to be a serious environmental stress, limiting the productivity of an organism. Genes conferring a high salt tolerant phenotype could be utilized as a non-antibiotic based, selectable marker for the genetic manipulation of strains of organisms. Additionally, it is recognized that by modification of an organism to improve particular characteristics, the use of the modified organism for the production of biofuels or other useful products is more commercially viable. To this end, strains of organisms can be developed which have improved characteristics, for example, increased salt tolerance over wild-type strains. Increasing the salt tolerance of a strain can facilitate its growth in media containing high salt concentrations, resulting in the production of commercially valuable products.

Therefore, there is a need to engineer photosynthetic organisms to be able to survive in conditions wherein contaminating organisms would normally not survive or have their growth rate decreased. One such condition is a saline environment. Thus, genetically engineering photosynthetic organisms to be salt tolerant would allow the organism to be grown in, for example, open ponds with high salinity, for the production of commercially valuable products.

### SUMMARY

1. An isolated polynucleotide capable of transforming a photosynthetic organism, wherein the polynucleotide comprises a nucleic acid sequence of SEQ ID NO: 1, SEQ ID NO: 3, SEQ ID NO: 4, SEQ ID NO: 6, SEQ ID NO: 8, SEQ ID NO: 10, SEQ ID NO: 12, SEQ ID NO: 14, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 26, SEQ ID NO: 29, SEQ ID NO: 31, SEQ ID NO: 33, SEQ ID NO: 35. SEQ ID NO: 37, SEQ ID NO: 39, SEQ ID NO:41, SEQ ID NO: 43, SEQ ID NO: 45, SEQ ID NO: 47, SEQ ID NO: 49, SEQ ID NO: 51, SEQ ID NO:53, SEQ ID NO: 55, SEQ ID NO: 57, SEQ ID NO: 59, or SEQ ID NO: 61. 2. The isolated polynucleotide of claim 1, wherein the polynucleotide comprises a nucleic acid sequence of SEQ ID NO: 26, SEQ ID NO: 31, SEQ ID NO: 35, SEQ ID NO: 39, SEQ ID NO: 43, SEQ ID NO: 47, SEQ ID NO: 51, SEQ ID NO: 55, or SEQ ID NO: 59. 3. The isolated polynucleotide of claim 1, wherein the photosynthetic organism is an alga. 4. The isolated polynucleotide of claim 3, wherein the alga is an alga from the genus Nannochloropsis or from the genus Chlamydomonas. 5. The isolated polynucleotide of claim 1, wherein the photosynthetic organism is a cyanobacteria. 6. The isolated polynucleotide of claim 1, wherein the photosynthetic organism is a Dunaliella. 7. The isolated polynucleotide of claim 1, wherein the photosynthetic organism is an obligatory phototroph and expression of the transporter does not alter the phototrophic state of the photosynthetic organism. 8. The isolated polynucleotide of claim 1, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

9. An isolated polynucleotide capable of transforming a photosynthetic organism, wherein the polynucleotide comprises a nucleic acid sequence of SEQ ID NO: 26, SEQ ID NO: 31, or SEQ ID NO: 35. 10. The isolated polynucleotide of claim 9, wherein the photosynthetic organism is an alga. 11. The isolated polynucleotide of claim 10, wherein the alga is an alga from the genus Nannochloropsis or from the genus Chlamydomonas. 12. The isolated polynucleotide of claim 9, wherein the photosynthetic organism is a cyanobacteria. 13. The isolated polynucleotide of claim 9, wherein the photosynthetic organism is a Dunaliella. 14. The isolated polynucleotide of claim 9, wherein the photosynthetic organism is an obligatory phototroph and expression of the transporter does not alter the phototrophic state of the photosynthetic organism. 15. The isolated polynucleotide of claim 9, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

16. An isolated polynucleotide capable of transforming a photosynthetic organism, comprising a nucleic acid encoding a protein that when expressed in the organism results in a salt tolerant organism as compared to a photosynthetic organism that is not transformed by the nucleic acid, wherein the protein comprises, (a) an amino acid sequence of SEQ ID NO: 2, SEQ ID NO: 5, SEQ ID NO: 7, SEQ ID NO: 9, SEQ ID NO: 11, SEQ ID NO: 13, SEQ ID NO: 16, SEQ ID NO: 25, SEQ ID NO: 27, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, or SEQ ID NO: 62; or (b) a homolog of the amino acid sequence of SEQ ID NO: 2, SEQ ID NO: 5, SEQ ID NO: 7, SEQ ID NO: 9, SEQ ID NO: 11, SEQ ID NO: 13, SEQ ID NO: 16, SEQ ID NO: 25, SEQ ID NO: 27, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, or SEQ ID NO: 62. 17. The isolated polynucleotide of claim 16, wherein the protein comprises, (a) an amino acid sequence of SEQ ID NO: 27, SEQ ID NO: 32, SEQ ID NO: 36, SEQ ID NO: 40, SEQ ID NO: 44, SEQ ID NO: 48, SEQ ID NO: 52, SEQ ID NO: 56, or SEQ ID NO: 60; or (b) a homolog of the amino acid sequence of SEQ ID NO: 27, SEQ ID NO: 32, SEQ ID NO: 36, SEQ ID NO: 40, SEQ ID NO: 44, SEQ ID NO: 48, SEQ ID NO: 52, SEQ ID NO: 56, or SEQ ID NO: 60. 18. The isolated polynucleotide of claim 16, wherein the photosynthetic organism is an alga. 19. The isolated polynucleotide of claim 18, wherein the alga is an alga from the genus Nannochloropsis or from the genus Chlamydomonas. 20. The isolated polynucleotide of claim 16, wherein the photosynthetic organism is a cyanobacteria. 21. The isolated polynucleotide of claim 16, wherein the photosynthetic organism is a Dunaliella. 22. The isolated polynucleotide of claim 16, wherein the photosynthetic organism is an obligatory phototroph and expression of the transporter does not alter the phototrophic state of the photosynthetic organism. 23. The isolated polynucleotide of claim 16, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

24. An isolated polynucleotide capable of transforming a photosynthetic organism, comprising a nucleic acid encoding a glutathione peroxidase (GPX) protein that when expressed in the organism results in a salt tolerant organism as compared to a photosynthetic organism that is not transformed by the nucleic acid, wherein the protein comprises, (a) an amino acid sequence of SEQ ID NO: 27, SEQ ID NO: 32, or SEQ ID NO: 36; or (b) a homolog of the amino acid sequence of SEQ ID NO: 27, SEQ ID NO: 32, or SEQ ID NO: 36. 25. The isolated polynucleotide of claim 24, wherein the photosynthetic organism is an alga. 26. The isolated polynucleotide of claim 25, wherein the alga is an alga from the genus Nannochloropsis or from the genus Chlamydomonas. 27. The isolated polynucleotide of claim 24, wherein the photosynthetic organism is a cyanobacteria. 28. The isolated polynucleotide of claim 24, wherein the photosynthetic organism is a Dunaliella. 29. The isolated polynucleotide of claim 24, wherein the photosynthetic organism is an obligatory phototroph and expression of the transporter does not alter the phototrophic state of the photosynthetic organism. 30. The isolated polynucleotide of claim 24, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

31. A vector comprising a polynucleotide capable of transforming a photosynthetic organism, comprising at least one nucleic acid sequence encoding a protein that when expressed in the photosynthetic organism, results in the photosynthetic organism becoming a salt tolerant photosynthetic organism as compared to a photosynthetic organism that is not transformed by the nucleic acid. 32. The vector of claim 31, wherein the nucleic acid is codon biased for a nuclear genome of the photosynthetic organism. 33. The vector of claim 31, wherein the nucleic acid is codon biased for a chloroplast genome of the photosynthetic organism. 34. The vector of claim 31, wherein the protein is a glutathione peroxidase (GPX) protein, an NHX protein, an SOS protein, or a BBC protein. 35. The vector of claim 34, wherein the GPX protein comprises an amino acid sequence of SEQ ID NO: 27, SEQ ID NO: 32, or SEQ ID NO: 36; or a homolog of SEQ ID NO: 27, SEQ ID NO: 32, or SEQ ID NO: 36. 36. The vector of claim 34, wherein the NHX protein comprises an amino acid sequence of SEQ ID NO: 40 or SEQ ID NO: 44; or a homolog of SEQ ID NO: 40 or SEQ ID NO: 44. 37. The vector of claim 34, wherein the SOS protein comprises an amino acid sequence of SEQ ID NO: 48; or a homolog of SEQ ID NO: 48. 38. The vector of claim 34, wherein the SOS protein comprises an amino acid sequence of SEQ ID NO: 52; or a homolog of SEQ ID NO: 52. 39. The vector of claim 31, wherein the protein comprises an amino acid sequence of SEQ ID NO: 56; or a homolog of SEQ ID NO: 56. 40. The vector of claim 31, wherein the protein comprises an amino acid sequence of SEQ ID NO: 60; or a homolog of SEQ ID NO: 60. 41. The vector of claim 31, wherein the protein is a voltage gated ion channel. 42. The vector of claim 31, wherein the protein is a protein that regulates the expression of a transporter. 43. The vector of claim 31, wherein the protein is a transporter 44. The vector of claim 43, wherein the transporter is an ion transporter. 45. The vector of claim 43, wherein the transporter transports Li+, Na+, or K+. 46. The vector of claim 43, wherein the transporter is an ATPase. 47. The vector of claim 46, wherein the ATPase is a Na+ ATPase, a Li+ ATPase, or a P-type ATPase. 48. The vector of claim 47, wherein the P-type ATPase is a yeast, plant, or algal P-type ATPase, or an ENA1 or a functional homolog of ENA1. 49. The vector of claim 43, wherein the transporter is an antiporter. 50. The vector of claim 49, wherein the antiporter is a Na+ antiporter. 51. The vector of claim 43, wherein the transporter is a CAX or a functional homolog of a CAX, a NHX or a functional homolog of a NHX, or a SOS or a functional homolog of a SOS, or a Nha protein or a functional homolog of a Nha protein, or a Nap protein or a functional homolog of a Nap protein. 52. The vector of claim 31, wherein the protein is a non-algal transporter, a non-algal protein that regulates the expression of a transporter, a vacuolar transporter, a protein that regulates expression of a vacuolar transporter, a H+-pyrophosphatase, a component of the SOS pathway, a BBC protein or a functional homolog of a BBC protein, or a SCSR protein or a functional homolog of a SCSR protein. 53. The vector of claim 52, wherein the H+-pyrophosphatase is AVP1 or a functional homolog of AVP1. 54. The vector of claim 52, wherein the component of the SOS pathway is SOS2, SOS3, or a functional homolog of SOS2 or SOS3. 55. The vector of claim 31, wherein the polynucleotide further comprises a second nucleic acid sequence. 56. The vector of claim 55, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 57. The vector of claim 56, wherein the antioxidant is glutathione peroxidase, ascorbate peroxidase, catalase, alternative oxidase, or superoxide dismutase. 58. The vector of claim 31, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 59. The vector of claim 31, wherein the nucleic acid sequence comprising a nucleotide sequence of SEQ ID NO: 26, SEQ ID NO: 31, SEQ ID NO: 35, SEQ ID NO: 39, SEQ ID NO: 43, SEQ ID NO: 47, SEQ ID NO: 51, SEQ ID NO: 55, or SEQ ID NO: 59. 60. The vector of any one of claims 31 to 59, wherein the nucleic acid sequence and/or second nucleic acid sequence are operably linked to a promoter. 61. The vector of claim 60, wherein the promoter is an RBCS promoter, an LHCP promoter, a tubulin promoter, or a pSAD promoter. 62. The vector of claim 60, wherein the promoter is a chimeric promoter. 63. The vector of claim 62, wherein the chimeric promoter is HSP70A/rbcS2. 64. The vector of claim 60, wherein the promoter is a constitutive promoter. 65. The vector of claim 60, wherein the promoter is an inducible promoter. 66. The vector of claim 60, wherein the promoter is a NIT1 promoter, a CYC6 promoter, or a CA1 promoter. 67. The vector of claim 31, wherein the polynucleotide further comprises a tag for isolation of purification of the transporter. 68. The vector of claim 67, wherein the tag is used to purify or isolate a protein or product. 69. The vector of claim 67, wherein the tag comprises an amino acid sequence of TGDYKDDDDKSGENLYFQGHNHRHKHTG (SEQ ID NO: 28) or comprises an amino acid sequence of PGDYKDDDDKSGENLYFQGHNHRHKHTG (SEQ ID NO:62). 70. The vector of claim 31, wherein the photosynthetic organism is an alga. 71. The vector of claim 70, wherein the nucleic acid is integrated into a chloroplast genome of the alga. 72. The vector of claim 70, wherein the nucleic acid is integrated into a nuclear genome of the alga. 73. The vector of claim 70, wherein the alga is an alga from the genus Nannochloropsis or from the genus Chlamydomonas. 74. The vector of claim 31, wherein the photosynthetic organism is a cyanobacteria. 75. The vector of claim 31, wherein the photosynthetic organism is a Dunaliella. 76. The vector of claim 31, wherein the photosynthetic organism is an obligatory phototroph and expression of the transporter does not alter the phototrophic state of the photosynthetic organism. 77. The vector of claim 31, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

8. A vector comprising a polynucleotide capable of transforming a photosynthetic organism, comprising at least one nucleic acid sequence encoding a glutathione peroxidase (GPX) protein that when expressed in the photosynthetic organism, results in the photosynthetic organism becoming a salt tolerant photosynthetic organism as compared to a photosynthetic organism that is not transformed by the nucleic acid. 79. The vector of claim 78, wherein the nucleic acid is codon biased for a nuclear genome of the photosynthetic organism. 80. The vector of claim 78, wherein the nucleic acid is codon biased for a chloroplast genome of the photosynthetic organism. 81. The vector of claim 78, wherein the nucleic acid comprises a nucleotide sequence of SEQ ID NO: 26, SEQ ID NO: 31, or SEQ ID NO: 35. 82. The vector of claim 78, wherein the polynucleotide further comprises a second nucleic acid sequence. 83. The vector of claim 82, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 84. The vector of claim 83, wherein the antioxidant is glutathione peroxidase, ascorbate peroxidase, catalase, alternative oxidase, or superoxide dismutase. 85. The vector of claim 78, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 86. The vector of any one of claims 78 to 85, wherein the nucleic acid sequence and/or second nucleic acid sequence are operably linked to a promoter. 87. The vector of claim 86, wherein the promoter is an RBCS promoter, an LHCP promoter, a tubulin promoter, or a pSAD promoter. 88. The vector of claim 86, wherein the promoter is a chimeric promoter. 89. The vector of claim 88, wherein the chimeric promoter is HSP70A/rbcS2. 90. The vector of claim 86, wherein the promoter is a constitutive promoter. 91. The vector of claim 86, wherein the promoter is an inducible promoter. 92. The vector of claim 86, wherein the promoter is a NIT1 promoter, a CYC6 promoter, or a CA1 promoter. 93. The vector of claim 78, wherein the polynucleotide further comprises a tag for isolation of purification of the transporter. 94. The vector of claim 93, wherein the tag is used to purify or isolate a protein or product. 95. The vector of claim 93, wherein the tag comprises an amino acid sequence of TGDYKDDDDKSGENLYFQGHNHRHKHTG (SEQ ID NO: 28) or comprises an amino acid sequence of PGDYKDDDDKSGENLYFQGHNHRHKHTG (SEQ ID NO: 62). 96. The vector of claim 78, wherein the GPX protein comprises an amino acid sequence of SEQ ID NO: 27, SEQ ID NO: 32, or SEQ ID NO: 36; or a homolog of SEQ ID NO: 27, SEQ ID NO: 32, or SEQ ID NO: 36. 97. The vector of claim 78, wherein the photosynthetic organism is an alga. 98. The vector of claim 97, wherein the nucleic acid is integrated into a chloroplast genome of the alga. 99. The vector of claim 97, wherein the nucleic acid is integrated into a nuclear genome of the alga. 100. The vector of claim 97, wherein the alga is an alga from the genus Nannochloropsis or from the genus Chlamydomonas. 101. The vector of claim 78, wherein the photosynthetic organism is a cyanobacteria. 102. The vector of claim 78, wherein the photosynthetic organism is a Dunaliella. 103. The vector of claim 78, wherein the photosynthetic organism is an obligatory phototroph and expression of the transporter does not alter the phototrophic state of the photosynthetic organism. 104. The vector of claim 78, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

105. An isolated photosynthetic organism comprising an exogenous polynucleotide capable of transforming the photosynthetic organism, wherein the exogenous polynucleotide comprises at least one nucleic acid sequence encoding a protein that when expressed in the photosynthetic organism, results in the photosynthetic organism becoming a salt tolerant photosynthetic organism as compared to a photosynthetic organism that is not transformed by the nucleic acid. 106. The isolated photosynthetic organism of claim 105, wherein the nucleic acid is codon biased for a nuclear genome of the photosynthetic organism. 107. The isolated photosynthetic organism of claim 105, wherein the nucleic acid is codon biased for a chloroplast genome of the photosynthetic organism. 108. The isolated photosynthetic organism of claim 105, wherein the protein is a glutathione peroxidase (GPX) protein, an NHX protein, an SOS protein, or a BBC protein. 109. The isolated photosynthetic organism of claim 108, wherein the GPX protein comprises an amino acid sequence of SEQ ID NO: 27, SEQ ID NO: 32, or SEQ ID NO: 36; or a homolog of SEQ ID NO: 27, SEQ ID NO: 32, or SEQ ID NO: 36. 110. The isolated photosynthetic organism of claim 108, wherein the NHX protein comprises an amino acid sequence of SEQ ID NO: 40 or SEQ ID NO: 44; or a homolog of SEQ ID NO: 40 or SEQ ID NO: 44. 111. The isolated photosynthetic organism of claim 108, wherein the SOS protein comprises an amino acid sequence of SEQ ID NO: 48; or a homolog of SEQ ID NO: 48. 112. The isolated photosynthetic organism of claim 108, wherein the SOS protein comprises an amino acid sequence of SEQ ID NO: 52; or a homolog of SEQ ID NO: 52. 113. The isolated photosynthetic organism of claim 105, wherein the protein comprises an amino acid sequence of SEQ ID NO: 56; or a homolog of SEQ ID NO: 56. 114. The isolated photosynthetic organism of claim 105, wherein the protein comprises an amino acid sequence of SEQ ID NO: 60; or a homolog of SEQ ID NO: 60. 115. The isolated photosynthetic organism of claim 105, wherein the protein is a voltage gated ion channel. 116. The isolated photosynthetic organism of claim 105, wherein the protein is a protein that regulates the expression of a transporter. 117. The isolated photosynthetic organism of claim 105, wherein the protein is a transporter. 118. The isolated photosynthetic organism of claim 117, wherein the transporter is an ion transporter. 119. The isolated photosynthetic organism of claim 117, wherein the transporter transports Li+, Na+, or K+. 120. The isolated photosynthetic organism of claim 117, wherein the transporter is an ATPase. 121. The isolated photosynthetic organism of claim 120, wherein the ATPase is a Na+ ATPase, a Li+ ATPase, or a P-type ATPase. 122. The isolated photosynthetic organism of claim 121, wherein the P-type ATPase is a yeast, plant, or algal P-type ATPase, or an ENA1 or a functional homolog of ENA1. 123. The isolated photosynthetic organism of claim 117, wherein the transporter is an antiporter. 124. The isolated photosynthetic organism of claim 123, wherein the antiporter is a Na+ antiporter. 125. The isolated photosynthetic organism of claim 117, wherein the transporter is a CAX or a functional homolog of a CAX, a NHX or a functional homolog of a NHX, or a SOS or a functional homolog of a SOS, or a Nha protein or a functional homolog of a Nha protein, or a Nap protein or a functional homolog of a Nap protein. 126. The isolated photosynthetic organism of claim 105, wherein the protein is a non-algal transporter, a non-algal protein that regulates the expression of a transporter, a vacuolar transporter, a protein that regulates expression of a vacuolar transporter, a H+-pyrophosphatase, a component of the SOS pathway, a BBC protein or a functional homolog of a BBC protein, or a SCSR protein or a functional homolog of a SCSR protein. 127. The isolated photosynthetic organism of claim 126, wherein the H+-pyrophosphatase is AVP1 or a functional homolog of AVP1. 128. The isolated photosynthetic organism of claim 126, wherein the component of the SOS pathway is SOS2, SOS3, or a functional homolog of SOS2 or SOS3. 129. The isolated photosynthetic organism of claim 105, wherein the nucleic acid sequence comprising a nucleotide sequence of SEQ ID NO: 26, SEQ ID NO: 31, SEQ ID NO: 35, SEQ ID NO: 39, SEQ ID NO: 43, SEQ ID NO: 47, SEQ ID NO: 51, SEQ ID NO: 55, or SEQ ID NO: 59. 130. The isolated photosynthetic organism of claim 105, wherein the polynucleotide further comprises a second nucleic acid sequence. 131. The isolated photosynthetic organism of claim 130, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 132. The isolated photosynthetic organism of claim 131, wherein the antioxidant is glutathione peroxidase, ascorbate peroxidase, catalase, alternative oxidase, or superoxide dismutase. 133. The isolated photosynthetic organism of claim 105, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 134. The isolated photosynthetic organism of any one of claims 105 to 133, wherein the nucleic acid sequence and/or second nucleic acid sequence are operably linked to a promoter. 135. The isolated photosynthetic organism of claim 134, wherein the promoter is an RBCS promoter, an LHCP promoter, a tubulin promoter, or a pSAD promoter. 136. The isolated photosynthetic organism of claim 134, wherein the promoter is a chimeric promoter. 137. The isolated photosynthetic organism of claim 136, wherein the chimeric promoter is HSP70A/rbcS2. 138. The isolated photosynthetic organism of claim 134, wherein the promoter is a constitutive promoter. 139. The isolated photosynthetic organism of claim 134, wherein the promoter is an inducible promoter. 140. The isolated photosynthetic organism of claim 134, wherein the promoter is a NIT1 promoter, a CYC6 promoter, or a CA1 promoter. 141. The isolated photosynthetic organism of claim 105, wherein the polynucleotide further comprises a tag for isolation of purification of the transporter. 142. The isolated photosynthetic organism of claim 141, wherein the tag is used to purify or isolate a protein or product. 143. The isolated photosynthetic organism of claim 141, wherein the tag comprises an amino acid sequence of TGDYKDDDDKSGENLYFQGHNHRHKHTG (SEQ ID NO: 28) or comprises an amino acid sequence of PGDYKDDDDKSGENLYFQGHNHRHKHTG (SEQ ID NO: 62). 144. The isolated photosynthetic organism of claim 105, wherein the photosynthetic organism is an alga. 145. The isolated photosynthetic organism of claim 144, wherein the nucleic acid is integrated into a chloroplast genome of the alga. 146. The isolated photosynthetic organism of claim 144, wherein the nucleic acid is integrated into a nuclear genome of the alga. 147. The isolated photosynthetic organism of claim 144, wherein the alga is an alga from the genus Nannochloropsis or from the genus Chlamydomonas. 148. The isolated photosynthetic organism of claim 105, wherein the photosynthetic organism is a cyanobacteria. 149. The isolated photosynthetic organism of claim 105, wherein the photosynthetic organism is a Dunaliella. 150. The isolated photosynthetic organism of claim 105, wherein the photosynthetic organism is an obligatory phototroph and expression of the transporter does not alter the phototrophic state of the photosynthetic organism. 151. The isolated photosynthetic organism of claim 105, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta. 152. The isolated photosynthetic organism of claim 105, wherein the photosynthetic organism is cultured or grown in a media. 153. The isolated photosynthetic organism of claim 152, wherein a concentration of at least 25 mM NaCl is added to the media. 154. The isolated photosynthetic organism of claim 152, wherein a concentration of at least 2 mM lithium is added to the media.

155. A isolated photosynthetic organism comprising an exogenous polynucleotide capable of transforming the photosynthetic organism, wherein the exogenous polynucleotide comprises at least one nucleic acid sequence encoding a glutathione peroxidase (GPX) protein that when expressed in the photosynthetic organism, results in the photosynthetic organism becoming a salt tolerant photosynthetic organism as compared to a photosynthetic organism that is not transformed by the nucleic acid. 156. The isolated photosynthetic organism of claim 155, wherein the nucleic acid is codon biased for a nuclear genome of the photosynthetic organism. 157. The isolated photosynthetic organism of claim 155, wherein the nucleic acid is codon biased for a chloroplast genome of the photosynthetic organism. 158. The isolated photosynthetic organism of claim 155, wherein the nucleic acid comprises a nucleotide sequence of SEQ ID NO: 26, SEQ ID NO: 31, or SEQ ID NO: 35. 159. The isolated photosynthetic organism of claim 155, wherein the GPX protein comprises an amino acid sequence of SEQ ID NO: 27, SEQ ID NO: 32, or SEQ ID NO: 36; or a homolog of ID NO: 27, SEQ ID NO: 32, or SEQ ID NO: 36. 160. The isolated photosynthetic organism of claim 155, wherein the polynucleotide further comprises a second nucleic acid sequence. 161. The isolated photosynthetic organism of claim 160, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 162. The isolated photosynthetic organism of claim 161, wherein the antioxidant is glutathione peroxidase, ascorbate peroxidase, catalase, alternative oxidase, or superoxide dismutase. 163. The isolated photosynthetic organism of claim 155, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 164. The isolated photosynthetic organism of any one of claims 155 to 163, wherein the nucleic acid sequence and/or second nucleic acid sequence are operably linked to a promoter. 165. The isolated photosynthetic organism of claim 164, wherein the promoter is an RBCS promoter, an LHCP promoter, a tubulin promoter, or a pSAD promoter. 166. The isolated photosynthetic organism of claim 164, wherein the promoter is a chimeric promoter. 167. The isolated photosynthetic organism of claim 166, wherein the chimeric promoter is HSP70A/rbcS2. 168. The isolated photosynthetic organism of claim 164, wherein the promoter is a constitutive promoter. 169. The isolated photosynthetic organism of claim 164, wherein the promoter is an inducible promoter. 170. The isolated photosynthetic organism of claim 164, wherein the promoter is a NIT1 promoter, a CYC6 promoter, or a CA promoter. 171. The isolated photosynthetic organism of claim 155, wherein the polynucleotide further comprises a tag for isolation of purification of the transporter. 172. The isolated photosynthetic organism of claim 171, wherein the tag is used to purify or isolate a protein or product. 173. The isolated photosynthetic organism of claim 171, wherein the tag comprises an amino acid sequence of TGDYKDDDDKSGENLYFQGHNHRHKHTG (SEQ ID NO: 28) or comprises an amino acid sequence of PGDYKDDDDKSGENLYFQGHNHRHKHTG (SEQ ID NO: 62). 174. The isolated photosynthetic organism of claim 155, wherein the photosynthetic organism is an alga. 175. The isolated photosynthetic organism of claim 174, wherein the nucleic acid is integrated into a chloroplast genome of the alga. 176. The isolated photosynthetic organism of claim 174, wherein the nucleic acid is integrated into a nuclear genome of the alga. 177. The isolated photosynthetic organism of claim 174, wherein the alga is an alga from the genus Nannochloropsis or from the genus Chlamydomonas. 178. The isolated photosynthetic organism of claim 155, wherein the photosynthetic organism is a cyanobacteria. 179. The isolated photosynthetic organism of claim 155, wherein the photosynthetic organism is a Dunaliella. 180. The isolated photosynthetic organism of claim 155, wherein the photosynthetic organism is an obligatory phototroph and expression of the transporter does not alter the phototrophic state of the photosynthetic organism. 181. The isolated photosynthetic organism of claim 155, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta. 182. The isolated photosynthetic organism of claim 155, wherein the photosynthetic organism is cultured or grown in a media. 183. The isolated photosynthetic organism of claim 182, wherein a concentration of at least 25 mM NaCl is added to the media. 184. The isolated photosynthetic organism of claim 182, wherein a concentration of at least 2 mM lithium is added to the media.

185. A method for increasing salt tolerance of a photosynthetic organism comprising, (a) transforming the photosynthetic organism with an exogenous nucleic acid sequence, wherein the nucleic acid sequence encodes a protein that when expressed in the photosynthetic organism, results in increased salt tolerance of the photosynthetic organism as compared to a photosynthetic organism that is not transformed by the nucleic acid. 186. The method of claim 185, wherein the nucleic acid is codon biased for a nuclear genome of the photosynthetic organism. 187. The method of claim 185, wherein the nucleic acid is codon biased for a chloroplast genome of the photosynthetic organism. 188. The method of claim 185, wherein the protein is a voltage gated ion channel. 189. The method of claim 185, wherein the protein is a protein that regulates the expression of a transporter. 190. The method of claim 185, wherein the protein is a transporter. 191. The method of claim 190, wherein the transporter is an ion transporter. 192. The method of claim 190, wherein the transporter transports Li+, Na+, or K+. 193. The method of claim 190, wherein the transporter is an ATPase. 194. The method of claim 193, wherein the ATPase is a Na+ ATPase, a Li+ ATPase, or a P-type ATPase. 195. The method of claim 194, wherein the P-type ATPase is a yeast, plant, or algal P-type ATPase, or an ENA1 or a functional homolog of ENA1. 196. The method of claim 190, wherein the transporter is an antiporter. 197. The method of claim 196, wherein the antiporter is a Na+ antiporter. 198. The method of claim 190, wherein the transporter is a CAX or a functional homolog of a CAX, a NHX or a functional homolog of a NHX, or a SOS or a functional homolog of a SOS, or a Nha protein or a functional homolog of a Nha protein, or a Nap protein or a functional homolog of a Nap protein. 199. The method of claim 185, wherein the protein is a non-algal transporter, a non-algal protein that regulates the expression of a transporter, a vacuolar transporter, a protein that regulates expression of a vacuolar transporter, a H+-pyrophosphatase, a component of the SOS pathway, a BBC protein or a functional homolog of a BBC protein, or a SCSR protein or a functional homolog of a SCSR protein. 200. The method of claim 199, wherein the H+-pyrophosphatase is AVP1 or a functional homolog of AVP1. 201. The method of claim 199, wherein the component of the SOS pathway is SOS2, SOS3, or a functional homolog of SOS2 or SOS3. 202. The method of claim 185, wherein the polynucleotide further comprises a second nucleic acid sequence. 203. The method of claim 202, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1.8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 204. The method of claim 203, wherein the antioxidant is glutathione peroxidase, ascorbate peroxidase, catalase, alternative oxidase, or superoxide dismutase. 205. The method of claim 185, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 206. The method of claim 185, wherein the nucleic acid sequence comprising a nucleotide sequence of SEQ ID NO: 26, SEQ ID NO: 31, SEQ ID NO: 35, SEQ ID NO: 39, SEQ ID NO: 43, SEQ ID NO: 47, SEQ ID NO: 51, SEQ ID NO: 55, or SEQ ID NO: 59. 207. The method of claim 185, wherein the protein is a glutathione peroxidase (GPX) protein, an NHX protein, an SOS protein, or a BBC protein. 208. The method of claim 207, wherein the GPX protein comprises an amino acid sequence of SEQ ID NO: 27, SEQ ID NO: 32, or SEQ ID NO: 36; or a homolog of SEQ ID NO: 27, SEQ ID NO: 32, or SEQ ID NO: 36. 209. The method of claim 207, wherein the NHX protein comprises an amino acid sequence of SEQ ID NO: 40 or SEQ ID NO: 44; or a homolog of SEQ ID NO: 40 or SEQ ID NO: 44. 210. The method of claim 207, wherein the SOS protein comprises an amino acid sequence of SEQ ID NO: 48; or a homolog of SEQ ID NO: 48. 211. The method of claim 207, wherein the SOS protein comprises an amino acid sequence of SEQ ID NO: 52; or a homolog of SEQ ID NO: 52. 212. The method of claim 185, wherein the protein comprises an amino acid sequence of SEQ ID NO: 56; or a homolog of SEQ ID NO: 56. 213. The method of claim 185, wherein the protein comprises an amino acid sequence of SEQ ID NO: 60; or a homolog of SEQ ID NO: 60. 214. The method of claim 185, wherein the photosynthetic organism is an alga. 215. The method of claim 214, wherein the nucleic acid is integrated into a chloroplast genome of the alga. 216. The method of claim 214, wherein the nucleic acid is integrated into a nuclear genome of the alga. 217. The method of claim 214, wherein the alga is an alga from the genus Nannochloropsis or from the genus Chlamydomonas. 218. The method of claim 185, wherein the photosynthetic organism is a cyanobacteria. 219. The method of claim 185, wherein the photosynthetic organism is a Dunaliella. 220. The method of claim 185, wherein the photosynthetic organism is an obligatory phototroph and expression of the transporter does not alter the phototrophic state of the photosynthetic organism. 221. The method of claim 185, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

222. A method for increasing salt tolerance of a photosynthetic organism comprising, (a) transforming the photosynthetic organism with an exogenous nucleic acid, wherein the nucleic acid sequence encodes a glutathione peroxidase (GPX) protein that when expressed in the photosynthetic organism, results in increased salt tolerance of the photosynthetic organism as compared to a photosynthetic organism that is not transformed by the nucleic acid. 223. The method of claim 222, wherein the nucleic acid is codon biased for a nuclear genome of the photosynthetic organism. 224. The method of claim 222, wherein the nucleic acid is codon biased for a chloroplast genome of the photosynthetic organism. 225. The method of claim 222, wherein the polynucleotide further comprises a second nucleic acid sequence. 226. The method of claim 225, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 227. The method of claim 226, wherein the antioxidant is glutathione peroxidase, ascorbate peroxidase, catalase, alternative oxidase, or superoxide dismutase. 228. The method of claim 222, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 229. The method of claim 222, wherein the nucleic acid comprises a nucleotide sequence of SEQ ID NO: 26, SEQ ID NO: 31, or SEQ ID NO: 35. 230. The method of claim 222, wherein the GPX protein comprises an amino acid sequence of SEQ ID NO: 27, SEQ ID NO: 32, or SEQ ID NO: 36; or a homolog of SEQ ID NO: 27, SEQ ID NO: 32, or SEQ ID NO: 36. 231. The method of claim 222, wherein the photosynthetic organism is an alga. 232. The method of claim 231, wherein the nucleic acid is integrated into a chloroplast genome of the alga. 233. The method of claim 231, wherein the nucleic acid is integrated into a nuclear genome of the alga. 234. The method of claim 231, wherein the alga is an alga from the genus Nannochloropsis or from the genus Chlamydomonas. 235. The method of claim 222, wherein the photosynthetic organism is a cyanobacteria. 236. The method of claim 222, wherein the photosynthetic organism is a Dunaliella. 237. The method of claim 222, wherein the photosynthetic organism is an obligatory phototroph and expression of the transporter does not alter the phototrophic state of the photosynthetic organism. 238. The method of claim 222, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

239. A method of selecting a photosynthetic organism capable of expressing a protein of interest, comprising: (a) introducing a first nucleic acid sequence encoding a first protein into the photosynthetic organism, wherein the first protein is the protein of interest; (b) introducing a second nucleic acid sequence encoding a second protein into the photosynthetic organism, wherein expression of the second protein confers salt tolerance to the photosynthetic organism as compared to a photosynthetic organism in which the second nucleic acid has not been introduced; (c) plating the photosynthetic organism on media or inoculating the photosynthetic organism in media, wherein the media comprises a concentration of salt that does not permit growth of the photosynthetic organism in which the second nucleic acid has not been introduced; (d) growing the photosynthetic organism; and (d) selecting at least one photosynthetic organism that grows on or in the medium. 240. The method of claim 239, wherein the second nucleic acid is codon biased for a nuclear genome of the photosynthetic organism. 241. The method of claim 239, wherein the second nucleic acid is codon biased for a chloroplast genome of the photosynthetic organism. 242. The method of claim 239, wherein the second protein is a voltage gated ion channel. 243. The method of claim 239, wherein the second protein is a protein that regulates the expression of a transporter. 244. The method of claim 239, wherein the second protein is a transporter. 245. The method of claim 244 wherein the transporter is an ion transporter. 246. The method of claim 244, wherein the transporter transports Li+, Na+, or K+. 247. The method of claim 244, wherein the transporter is an ATPase. 248. The method of claim 247, wherein the ATPase is a Na+ ATPase, a Li+ ATPase, or a P-type ATPase. 249. The method of claim 248, wherein the P-type ATPase is a yeast, plant, or algal P-type ATPase, or an ENA1 or a functional homolog of ENA1. 250. The method of claim 244, wherein the transporter is an antiporter. 251. The method of claim 250, wherein the antiporter is a Na+ antiporter. 252. The method of claim 244, wherein the transporter is a CAX or a functional homolog of a CAX, a NHX or a functional homolog of a NHX, or a SOS or a functional homolog of a SOS, or a Nha protein or a functional homolog of a Nha protein, or a Nap protein or a functional homolog of a Nap protein. 253. The method of claim 239, wherein the second protein is a non-algal transporter, a non-algal protein that regulates the expression of a transporter, a vacuolar transporter, a protein that regulates expression of a vacuolar transporter, a H+-pyrophosphatase, a component of the SOS pathway, a BBC protein or a functional homolog of a BBC protein, or a SCSR protein or a functional homolog of a SCSR protein. 254. The method of claim 253, wherein the H+-pyrophosphatase is AVP1 or a functional homolog of AVP1. 255. The method of claim 253, wherein the component of the SOS pathway is SOS2, SOS3, or a functional homolog of SOS2 or SOS3. 256. The method of claim 239, wherein the second nucleic acid sequence comprises a nucleotide sequence of SEQ ID NO: 26, SEQ ID NO: 31, SEQ ID NO: 35. SEQ ID NO: 39, SEQ ID NO: 43, SEQ ID NO: 47, SEQ ID NO: 51, SEQ ID NO: 55, or SEQ ID NO: 59. 257. The method of claim 239, wherein the second protein comprises, (a) an amino acid sequence of SEQ ID NO: 27, SEQ ID NO: 32, SEQ ID NO: 36, SEQ ID NO: 40, SEQ ID NO: 44, SEQ ID NO: 48, SEQ ID NO: 52, SEQ ID NO: 56, or SEQ ID NO: 60; or (b) a homolog of the amino acid sequence of SEQ ID NO: 27, SEQ ID NO: 32, SEQ ID NO: 36, SEQ ID NO: 40, SEQ ID NO: 44, SEQ ID NO: 48, SEQ ID NO: 52, SEQ ID NO: 56, or SEQ ID NO: 60. 258. The method of claim 239, wherein the photosynthetic organism is an alga. 259. The method of claim 258, wherein the nucleic acid is integrated into a chloroplast genome of the alga. 260. The method of claim 258, wherein the nucleic acid is integrated into a nuclear genome of the alga. 261. The method of claim 239, wherein the alga is an alga from the genus Nannochloropsis or from the genus Chlamydomonas. 262. The method of claim 239, wherein the photosynthetic organism is a cyanobacteria. 263. The method of claim 239, wherein the photosynthetic organism is a Dunaliella. 264. The method of claim 239, wherein the photosynthetic organism is an obligatory phototroph and expression of the transporter does not alter the phototrophic state of the photosynthetic organism. 265. The method of claim 239, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta. 266. The method of claim 239, wherein the first nucleic acid sequence encodes a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product.

267. A method of selecting a photosynthetic organism capable of expressing a protein of interest, comprising: (a) introducing a first nucleic acid sequence encoding a first protein into the photosynthetic organism, wherein the first protein is the protein of interest: (b) introducing a second nucleic acid sequence encoding a second protein into the photosynthetic organism, wherein expression of the second protein confers salt tolerance to the photosynthetic organism as compared to a photosynthetic organism in which the second nucleic acid has not been introduced, and wherein the second protein is a glutathione peroxidase (GPX) protein; (c) plating the photosynthetic organism on media or inoculating the photosynthetic organism in media, wherein the media comprises a concentration of salt that does not permit growth of the photosynthetic organism in which the second nucleic acid has not been introduced; (d) growing the photosynthetic organism; and (e) selecting at least one photosynthetic organism that grows on or in the medium. 268. The method of claim 267, wherein the second nucleic acid is codon biased for a nuclear genome of the photosynthetic organism. 269. The method of claim 267, wherein the second nucleic acid is codon biased for a chloroplast genome of the photosynthetic organism. 270. The method of claim 267, wherein the second nucleic acid sequence comprises a nucleotide sequence of SEQ ID NO: 26, SEQ ID NO: 31, or SEQ ID NO: 35. 271. The method of claim 267, wherein the second protein comprises, (a) an amino acid sequence of SEQ ID NO: 27, SEQ ID NO: 32, or SEQ ID NO: 36; or (b) a homolog of the amino acid sequence of SEQ ID NO: 27, SEQ ID NO: 32, or SEQ ID NO: 36. 272. The method of claim 267, wherein the photosynthetic organism is an alga. 273. The method of claim 272, wherein the nucleic acid is integrated into a chloroplast genome of the alga. 274. The method of claim 272, wherein the nucleic acid is integrated into a nuclear genome of the alga. 275. The method of claim 272, wherein the alga is an alga from the genus Nannochloropsis or from the genus Chlamydomonas. 276. The method of claim 267, wherein the photosynthetic organism is a cyanobacteria. 277. The method of claim 267, wherein the photosynthetic organism is a Dunaliella. 278. The method of claim 267, wherein the photosynthetic organism is an obligatory phototroph and expression of the transporter does not alter the phototrophic state of the photosynthetic organism. 279. The method of claim 267, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

280. A method for producing one or more products, comprising: (a) growing a photosynthetic organism transformed with a polynucleotide comprising a nucleic acid encoding a protein that when expressed in the photosynthetic organism, results in the photosynthetic organism becoming a salt tolerant photosynthetic organism as compared to a photosynthetic organism that is not transformed by the nucleic acid; and (b) harvesting one or more products from the photosynthetic organism. 281. The method of claim 280, wherein the nucleic acid is codon biased for a nuclear genome of the photosynthetic organism. 282. The method of claim 280, wherein the nucleic acid is codon biased for a chloroplast genome of the photosynthetic organism. 283. The method of claim 280, wherein the protein is a voltage gated ion channel. 284. The method of claim 280, wherein the protein is a protein that regulates the expression of a transporter. 285. The method of claim 280, wherein the protein is a transporter. 286. The method of claim 285, wherein the transporter is an ion transporter. 287. The method of claim 285, wherein the transporter transports Li+, Na+, or K+. 288. The method of claim 285, wherein the transporter is an ATPase. 289. The method of claim 288, wherein the ATPase is a Na+ ATPase, a Li+ ATPase, or a P-type ATPase. 290. The method of claim 289, wherein the P-type ATPase is a yeast, plant, or algal P-type ATPase, or an ENA1 or a functional homolog of ENA1. 291. The method of claim 285, wherein the transporter is an antiporter. 292. The method of claim 291, wherein the antiporter is a Na+ antiporter. 293. The method of claim 285, wherein the transporter is a CAX or a functional homolog of a CAX, a NHX or a functional homolog of a NHX, or a SOS or a functional homolog of a SOS, or a Nha protein or a functional homolog of a Nha protein, or a Nap protein or a functional homolog of a Nap protein. 294. The method of claim 280, wherein the protein is a non-algal transporter, a non-algal protein that regulates the expression of a transporter, a vacuolar transporter, a protein that regulates expression of a vacuolar transporter, a H+-pyrophosphatase, a component of the SOS pathway, a BBC protein or a functional homolog of a BBC protein, or a SCSR protein or a functional homolog of a SCSR protein. 295. The method of claim 294, wherein the H+-pyrophosphatase is AVP1 or a functional homolog of AVP1. 296. The method of claim 294, wherein the component of the SOS pathway is SOS2, SOS3, or a functional homolog of SOS2 or SOS3. 297. The method of claim 280, wherein the polynucleotide further comprises a second nucleic acid sequence. 298. The method of claim 297, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-ar-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 299. The method of claim 298, wherein the antioxidant is glutathione peroxidase, ascorbate peroxidase, catalase, alternative oxidase, or superoxide dismutase. 300. The method of claim 280, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 301. The method of claim 280, wherein the nucleic acid sequence comprises a nucleotide sequence of SEQ ID NO: 26, SEQ ID NO: 31, SEQ ID NO: 35, SEQ ID NO: 39, SEQ ID NO: 43, SEQ ID NO: 47, SEQ ID NO: 51, SEQ ID NO: 55, or SEQ ID NO: 59. 302. The method of claim 280, wherein the protein comprises an amino acid sequence of SEQ ID NO: 27, SEQ ID NO: 32, SEQ ID NO: 36, SEQ ID NO: 40, SEQ ID NO: 44, SEQ ID NO: 48, SEQ ID NO: 52, SEQ ID NO: 56, or SEQ ID NO: 60. 303. The method of claim 280, wherein the photosynthetic organism is an alga. 304. The method of claim 303, wherein the nucleic acid is integrated into a chloroplast genome of the alga. 305. The method of claim 303, wherein the nucleic acid is integrated into a nuclear genome of the alga. 306. The method of claim 303, wherein the alga is an alga from the genus Nannochloropsis or from the genus Chlamydomonas. 307. The method of claim 280, wherein the photosynthetic organism is a cyanobacteria. 308. The method of claim 280, wherein the photosynthetic organism is a Dunaliella. 309. The method of claim 280, wherein the photosynthetic organism is an obligatory phototroph and expression of the transporter does not alter the phototrophic state of the photosynthetic organism. 310. The method of claim 280, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

311. A method for producing one or more products, comprising: (a) growing a photosynthetic organism transformed with a polynucleotide comprising a nucleic acid encoding a glutathione peroxidase (GPX) protein that when expressed in the photosynthetic organism, results in the photosynthetic organism becoming a salt tolerant photosynthetic organism as compared to a photosynthetic organism that is not transformed by the nucleic acid; and (b) harvesting one or more products from the photosynthetic organism. 312. The method of claim 311, wherein the nucleic acid is codon biased for a nuclear genome of the photosynthetic organism. 313. The method of claim 311, wherein the nucleic acid is codon biased for a chloroplast genome of the photosynthetic organism. 314. The method of claim 311, wherein the polynucleotide further comprises a second nucleic acid sequence. 315. The method of claim 314, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 316. The method of claim 315, wherein the antioxidant is glutathione peroxidase, ascorbate peroxidase, catalase, alternative oxidase, or superoxide dismutase. 317. The method of claim 311, wherein the nucleic acid sequence encodes for an ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 318. The method of claim 311, wherein the nucleic acid sequence comprises a nucleotide sequence of SEQ ID NO: 26, SEQ ID NO: 31, or SEQ ID NO: 35. 319. The method of claim 311, wherein the protein comprises an amino acid sequence of SEQ ID NO: 27, SEQ ID NO: 32, or SEQ ID NO: 36. 320. The method of claim 311, wherein the photosynthetic organism is an alga. 321. The method of claim 320, wherein the nucleic acid is integrated into a chloroplast genome of the alga. 322. The method or claim 320, wherein the nucleic acid is integrated into a nuclear genome of the alga. 323. The method of claim 320, wherein the alga is an alga from the genus Nannochloropsis or from the genus Chlamydomonas. 324. The method of claim 311, wherein the photosynthetic organism is a cyanobacteria. 325. The method of claim 311, wherein the photosynthetic organism is a Dunaliella. 326. The method of claim 311, wherein the photosynthetic organism is an obligatory phototroph and expression of the transporter does not alter the phototrophic state of the photosynthetic organism. 327. The method of claim 311, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

328. An isolated polynucleotide capable of transforming a photosynthetic organism, wherein the polynucleotide comprises a nucleic acid sequence of SEQ ID NO: 55. 329. The isolated polynucleotide of claim 328, wherein the photosynthetic organism is an alga. 330. The isolated polynucleotide of claim 329, wherein the alga is an alga from the genus Nannochloropsis or from the genus Chlamydomonas. 331. The isolated polynucleotide of claim 328, wherein the photosynthetic organism is a cyanobacteria. 332. The isolated polynucleotide of claim 328, wherein the photosynthetic organism is a Dunaliella. 333. The isolated polynucleotide of claim 328, wherein the photosynthetic organism is an obligatory phototroph and expression of the transporter does not alter the phototrophic state of the photosynthetic organism. 334. The isolated polynucleotide of claim 328, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

335. An isolated polynucleotide capable of transforming a photosynthetic organism, wherein the polynucleotide comprises a nucleic acid encoding a protein that when expressed in the organism results in a salt tolerant organism as compared to a photosynthetic organism that is not transformed by the nucleic acid, wherein the protein comprises, an amino acid sequence of SEQ ID NO: 56 or a homolog of the amino acid sequence of SEQ ID NO: 56. 336. The isolated polynucleotide of claim 335, wherein the photosynthetic organism is an alga. 337. The isolated polynucleotide of claim 336, wherein the alga is an alga from the genus Nannochloropsis or from the genus Chlamydomonas. 338. The isolated polynucleotide of claim 335, wherein the photosynthetic organism is a cyanobacteria. 339. The isolated polynucleotide of claim 355, wherein the photosynthetic organism is a Dunaliella. 340. The isolated polynucleotide of claim 355, wherein the photosynthetic organism is an obligatory phototroph and expression of the transporter does not alter the phototrophic state of the photosynthetic organism. 341. The isolated polynucleotide of claim 335, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

342. A vector comprising a polynucleotide capable of transforming a photosynthetic organism, wherein the polynucleotide comprises at least one nucleic acid sequence encoding a protein comprising an amino acid sequence of SEQ ID NO: 56, wherein when the protein is expressed in the photosynthetic organism, the photosynthetic organism becomes a salt tolerant photosynthetic organism as compared to a photosynthetic organism that is not transformed by the nucleic acid. 343. The vector of claim 342, wherein the polynucleotide further comprises a second nucleic acid sequence. 344. The vector of claim 343, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 345. The vector of claim 344, wherein the antioxidant is glutathione peroxidase, ascorbate peroxidase, catalase, alternative oxidase, or superoxide dismutase. 346. The vector of claim 342, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 347. The vector of claim 342, wherein the nucleic acid sequence comprises the nucleotide sequence of SEQ ID NO: 55. 348. The vector of claim 342, wherein the photosynthetic organism is an alga. 349. The vector of claim 348, wherein the alga is an alga from the genus Nannochloropsis or from the genus Chlamydomonas. 350. The vector of claim 342, wherein the photosynthetic organism is a cyanobacteria. 351. The vector of claim 342, wherein the photosynthetic organism is a Dunaliella. 352. The vector of claim 342, wherein the photosynthetic organism is an obligatory phototroph and expression of the transporter does not alter the phototrophic state of the photosynthetic organism. 353. The vector of claim 342, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta. 354. The vector of any one of claims 342 to 353, wherein the nucleic acid sequence and/or second nucleic acid sequence are operably linked to a promoter. 355. The vector of claim 354, wherein the promoter is an RBCS promoter, an LHCP promoter, a tubulin promoter, or a pSAD promoter. 356. The vector of claim 354, wherein the promoter is a chimeric promoter. 357. The vector of claim 356, wherein the chimeric promoter is HSP70A/rbcS2. 358. The vector of claim 354, wherein the promoter is a constitutive promoter. 359. The vector of claim 354, wherein the promoter is an inducible promoter. 360. The vector of claim 354, wherein the promoter is a NIT1 promoter, a CYC6 promoter, or a CA1 promoter. 361. The vector of claim 354, wherein the polynucleotide further comprises a tag for isolation of purification of the transporter. 362. The vector of claim 361, wherein the tag is used to purify or isolate a protein or product. 363. The vector of claim 361, wherein the tag comprises an amino acid sequence of TGDYKDDDDKSGENLYFQGHNHRHKHTG (SEQ ID NO: 28) or comprises an amino acid sequence of PGDYKDDDDKSGENLYFQGHNHRHKHTG (SEQ ID NO: 62).

364. An isolated photosynthetic organism comprising an exogenous polynucleotide capable of transforming the photosynthetic organism, wherein the exogenous polynucleotide comprises at least one nucleic acid sequence encoding a protein comprising an amino acid sequence of SEQ ID NO: 56, wherein when the protein is expressed in the photosynthetic organism, the photosynthetic organism becomes a salt tolerant photosynthetic organism as compared to a photosynthetic organism that is not transformed by the nucleic acid. 365. The isolated photosynthetic organism of claim 364, wherein the polynucleotide further comprises a second nucleic acid sequence. 366. The isolated photosynthetic organism of claim 365, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 367. The isolated photosynthetic organism of claim 366, wherein the antioxidant is glutathione peroxidase, ascorbate peroxidase, catalase, alternative oxidase, or superoxide dismutase. 368. The isolated photosynthetic organism of claim 364, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 369. The isolated photosynthetic organism of claim 364, wherein the nucleic acid sequence comprises the nucleotide sequence of SEQ ID NO: 55. 370. The isolated photosynthetic organism of claim 364, wherein the photosynthetic organism is an alga. 371. The isolated photosynthetic organism of claim 370, wherein the alga is an alga from the genus Nannochloropsis or from the genus Chlamydomonas. 372. The isolated photosynthetic organism of claim 364, wherein the photosynthetic organism is a cyanobacteria. 373. The isolated photosynthetic organism of claim 364, wherein the photosynthetic organism is a Dunaliella. 374. The isolated photosynthetic organism of claim 364, wherein the photosynthetic organism is an obligatory phototroph and expression of the transporter does not alter the phototrophic state of the photosynthetic organism. 375. The isolated photosynthetic organism of claim 364, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta.

376. A method for increasing salt tolerance of a photosynthetic organism comprising, (a) transforming the photosynthetic organism with an exogenous polynucleotide sequence comprising a nucleic acid sequence, wherein the nucleic acid sequence encodes a protein comprising an amino acid sequence of SEQ ID NO: 56, wherein expression of the protein in the photosynthetic organism results in increased salt tolerance of the photosynthetic organism as compared to a photosynthetic organism that is not transformed by the nucleic acid. 377. The method of claim 376, wherein the polynucleotide further comprises a second nucleic acid sequence. 378. The method of claim 377, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 379. The method of claim 378, wherein the antioxidant is glutathione peroxidase, ascorbate peroxidase, catalase, alternative oxidase, or superoxide dismutase. 380. The method of claim 376, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 381. The method of claim 376, wherein the tolerance of the photosynthetic organism is at least twice, at least three times, or at least four times that of the photosynthetic organism that is not transformed by the nucleic acid. 382. The method of claim 376, wherein the nucleic acid sequence comprises the nucleotide sequence of SEQ ID NO: 55. 383. The method of claim 376, wherein the photosynthetic organism is an alga. 384. The method of claim 383, wherein the alga is an alga from the genus Nannochloropsis or from the genus Chlamydomonas. 385. The method of claim 376, wherein the photosynthetic organism is a cyanobacteria. 386. The method of claim 376, wherein the photosynthetic organism is a Dunaliella. 387. The method of claim 376, wherein the photosynthetic organism is an obligatory phototroph and expression of the transporter does not alter the phototrophic state of the photosynthetic organism. 388. The method of claim 376, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta. 389. The method of any one of claims 376 to 388, wherein the nucleic acid is integrated into a chloroplast genome of the alga. 390. The method of any one of claims 376 to 388, wherein the nucleic acid is integrated into a nuclear genome of the alga.

391. A method of selecting a photosynthetic organism capable of expressing a protein of interest, comprising: (a) introducing a first nucleic acid sequence encoding a first protein into the photosynthetic organism, wherein the first protein is the protein of interest: (b) introducing a second nucleic acid sequence encoding a second protein into the photosynthetic organism, wherein expression of the second protein confers salt tolerance to the photosynthetic organism as compared to a photosynthetic organism in which the second nucleic acid has not been introduced, and wherein the second protein comprises an amino acid sequence of SEQ ID NO: 56; (c) plating the photosynthetic organism on media or inoculating the photosynthetic organism in media, wherein the media comprises a concentration of salt that does not permit growth of the photosynthetic organism in which the second nucleic acid has not been introduced; (d) growing the photosynthetic organism; and (e) selecting at least one photosynthetic organism that grows on or in the medium. 392. The method of claim 391, wherein the second nucleic acid sequence comprises the nucleotide sequence of SEQ ID NO: 55. 393. The method of claim 391, wherein the photosynthetic organism is an alga. 394. The method of claim 393, wherein the alga is an alga from the genus Nannochloropsis or from the genus Chlamydomonas. 395. The method of claim 391, wherein the photosynthetic organism is a cyanobacteria. 396. The method of claim 391, wherein the photosynthetic organism is a Dunaliella. 397. The method of claim 391, wherein the photosynthetic organism is an obligatory phototroph and expression of the transporter does not alter the phototrophic state of the photosynthetic organism. 398. The method of claim 391, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta. 399. The method of any one of claims 391 to 398, wherein the nucleic acid is integrated into a chloroplast genome of the alga. 400. The method of any one of claims 391 to 398, wherein the nucleic acid is integrated into a nuclear genome of the alga.

401. A method for producing one or more products, comprising: (a) growing a photosynthetic organism transformed with a polynucleotide comprising a nucleic acid encoding a protein comprising an amino acid sequence of SEQ ID NO: 56, wherein when the protein is expressed in the photosynthetic organism the photosynthetic organism becomes a salt tolerant photosynthetic organism as compared to a photosynthetic organism that is not transformed by the nucleic acid; and (b) harvesting one or more products from the photosynthetic organism. 402. The method of claim 401, wherein the polynucleotide further comprises a second nucleic acid sequence. 403. The method of claim 402, wherein the second nucleic acid sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a protein that confers salt tolerance to an organism, a BBC protein or a functional homolog of a BBC protein, or a SCSR protein or a functional homolog of a SCSR protein. 404. The method of claim 403, wherein the antioxidant is glutathione peroxidase, ascorbate peroxidase, catalase, alternative oxidase, or superoxide dismutase. 405. The method of claim 401, wherein the nucleic acid sequence encodes for a ATPase and the second nucleic acid sequence encodes for an antiporter, or the nucleic acid sequence encodes for a plasma membrane ATPase and the second nucleic acid sequence encodes for a vacuolar antiporter, or the nucleic acid sequence encodes for a plasma membrane ATPase and the second nucleic acid sequence encodes for a plasma membrane antiporter, or the nucleic acid sequence encodes for a H+-pyrophosphatase and the second nucleic acid sequence encodes for an antiporter, or the nucleic acid sequence encodes for a vacuolar H+-pyrophosphatase and the second nucleic acid sequence encodes for a vacuolar antiporter, or the nucleic acid sequence encodes for a transporter or a protein that regulates expression of a transporter, or a protein that confers salt tolerance to an organism, and the second nucleic acid sequence encodes for a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product. 406. The method of claim 401, wherein the nucleic acid comprises the nucleotide sequence of SEQ ID NO: 55. 407. The method of claim 401, wherein the photosynthetic organism is an alga. 408. The method of claim 407, wherein the alga is an alga from the genus Nannochloropsis or from the genus Chlamydomonas. 409. The method of claim 401, wherein the photosynthetic organism is a cyanobacteria. 410. The method of claim 401, wherein the photosynthetic organism is a Dunaliella. 411. The method of claim 401, wherein the photosynthetic organism is an obligatory phototroph and expression of the transporter does not alter the phototrophic state of the photosynthetic organism. 412. The method of claim 401, wherein the photosynthetic organism is a cyanophyta, a rhodophyta, a chlorophyta, a phaeophyta, a baccilariophyta, a chrysophyta, a heterokontophyta, a tribophyta, a glaucophyta, a chlorarachniophyta, a euglenophyta, a haptophyta, a cryptophyla, or a dinophyta. 413. The method of any one of claims 401 to 411, wherein the nucleic acid is integrated into a chloroplast genome of the alga. 414. The method of any one of claims 401 to 411, wherein the nucleic acid is integrated into a nuclear genome of the alga. 415. The method of any one of claims 401 to 414, wherein the product is a therapeutic protein, a nutritional protein, an industrial enzyme, a protein that participates in or promotes the synthesis of at least one nutritional product, therapeutic product, commercial product, or fuel product, or a protein that facilitates the isolation of at least one nutritional product, therapeutic product, commercial product, or fuel product.

In one aspect, the present disclosure provides an expression vector comprising a polynucleotide encoding a transporter or a protein that regulates the expression of a transporter, wherein the polynucleotide is codon biased for the nuclear genome of an algal host, wherein the transporter does not transport a reduced carbon source. In another aspect, the present disclosure provides an expression vector comprising a polynucleotide encoding a transporter or a protein that regulates the expression of a transporter, operably linked to an exogenous or endogenous promoter that functions in an algal cell, wherein the transporter does not transport a reduced carbon source. In another aspect, the present disclosure provides an expression vector comprising a polynucleotide encoding a non-algal transporter or a non-algal protein that regulates the expression of a transporter, operably linked to an algal regulatory sequence, wherein the transporter does not transport a reduced carbon source. The transporter can be an ion transporter. In some embodiments, the polynucleotide is operably linked an algal promoter. The promoter may be an RBCS promoter, an LHCP promoter, a tubulin promoter, or a PsaD promoter. In some embodiments, the promoter is an inducible promoter, for example, a NIT1 promoter, a CYC6 promoter or a CA1 promoter. In some embodiments, the polynucleotide comprises a sequence that encodes an ion transporter. The transporter can be an ATPase such as a Na+ ATPase or a P-type ATPase. In some embodiments, the P-type ATPase is a yeast, plant, or algal P-type ATPase. The P-type ATPase may be ENA1 or a functional homolog thereof. In some embodiments, the ion transporter is an antiporter. The antiporter is a Na+ antiporter. Examples of the antiporter include but are not limited to NHX1 or a functional homolog thereof, SOS1 or a functional homolog thereof. In some embodiments, the exogenous or endogenous polynucleotide encodes an H+-pyrophosphatase. The H+-pyrophosphatase can be AVP1 or a functional homolog thereof. In some embodiments, the polynucleotide encodes a protein that regulates the expression of a transporter. The polynucleotide may encode at least one component of the SOS pathway. Component of the SOS pathway can be SOS2, SOS3, or a functional homolog thereof.

In another aspect, the present disclosure provides a transgenic alga comprising an exogenous or endogenous polynucleotide encoding an ATPase ion transporter. In another aspect, the present disclosure provides a transgenic alga comprising an exogenous or endogenous polynucleotide encoding a transporter or a protein that regulates expression of a transporter. In another aspect, the present disclosure provides a transgenic alga comprising an exogenous or endogenous polynucleotide encoding a vacuolar transporter. In another aspect, the present disclosure provides a transgenic alga comprising an exogenous or endogenous polynucleotide encoding a transporter or a protein that regulates expression of a transporter, wherein the transporter does not transport a catabolizable carbon source, further wherein the polynucleotide is codon biased for the nuclear genome of the algal cell. In another aspect, the present disclosure provides a transgenic alga comprising an exogenous or endogenous polynucleotide encoding an ion transporter or a protein that regulates expression of an ion transporter, wherein the polynucleotide is codon biased for the nuclear genome of the algal cell. The present disclosure also provides a transgenic alga comprising an exogenous or endogenous polynucleotide encoding a transporter or a protein that regulates expression of a transporter, wherein the algal cell is an obligatory phototroph and the exogenous or endogenous polynucleotide does not alter the phototrophic state of the alga. In another aspect, the present disclosure provides a transgenic alga comprising two or more exogenous or endogenous polynucleotides, wherein at least one of the exogenous or endogenous polynucleotides encodes a transporter or a protein that regulates expression of a transporter, wherein the transporter does not transport a catabolizable carbon source. In yet another aspect, the present disclosure provides a transgenic alga comprising two or more exogenous or endogenous polynucleotides, wherein at least one of the exogenous or endogenous polynucleotides encodes an ion transporter or a protein that regulates expression of an ion transporter. In yet another aspect, the present disclosure provides a transgenic eukaryotic unicellular alga comprising an exogenous or endogenous polynucleotide encoding a transporter or a protein that regulates expression of a transporter, wherein the transporter does not transport a catabolizable carbon source. In still another aspect, the present disclosure provides a transgenic eukaryotic unicellular alga comprising an exogenous or endogenous polynucleotide encoding an ion transporter or a protein that regulates expression of an ion transporter.

In any of the subject compositions disclosed herein, the transgenic alga may be a cyanophyta, a rhodophyta, chlorophyta, phaeophyta, baccilariophyta, chrysophyta, heterokontophyta, tribophyta, glaucophyta, chlorarachniophyta, euglenophyta, haptophyta, cryptophyla, or dinophyta species. In some embodiments, the transgenic alga is a rhodophyta, chlorophyta, rhodophyta, phaeophyta, baccilariophyta, chrysophyta, heterokontophyta, tribophyta, glaucophyta, chlorarachniophyta, euglenophyta, haptophyta, cryptophyla, or dinophyta species. In some embodiments, the algal cell has increased salt tolerance with respect to an algal cell that does not comprise the exogenous or endogenous polynucleotide encoding the transporter. In some embodiments, the tolerance is at least twice, at least three times, or at least four times that of a wild-type alga. In some embodiments, the exogenous or endogenous polynucleotide is operably linked to an algal promoter. In some embodiments, the algal promoter is an inducible promoter. In some embodiments, the algal promoter is a constitutive promoter. In some embodiments, the promoter is a chimeric promoter. In some embodiments, the transporter transports Li+, Na+, or K+. In some embodiments, the transporter is an ATPase. In some embodiments, the ATPase is a Na+ ATPase, a Li+ ATPase, or a P-type ATPase. In some embodiments, the P-type ATPase is ENA1 or a functional homolog thereof. In some embodiments, the transporter is an antiporter. In some embodiments, the transporter is a Na+ antiporter. In some embodiments, the antiporter is a CAX antiporter, a NHX antiporter, or a functional homolog thereof. In some embodiments, the transporter is an SOS1 protein, an Nha protein, or an Nap protein, or a functional homolog of any of the above. In some embodiments, the exogenous or endogenous polynucleotide encodes a H+-pyrophosphatase. In some embodiments, the H+-pyrophosphatase is AVP1 or a functional homolog thereof. In some embodiments, the exogenous or endogenous polynucleotide encodes a protein that regulates the expression of a transporter. In some embodiments, the exogenous or endogenous polynucleotide encodes SOS2, SOS3, or a functional homolog thereof.

In some embodiments, the transgenic alga comprises two or more exogenous or endogenous polynucleotides, wherein each of the exogenous or endogenous polynucleotides encodes an ATPase, an antiporter, or an H+-pyrophosphatase. In some embodiments, the transgenic alga comprises a first exogenous or endogenous polynucleotide encoding an ATPase and a second exogenous or endogenous polynucleotide encoding an antiporter. In some embodiments, the transgenic alga comprises a first exogenous or endogenous polynucleotide encoding a plasma membrane ATPase and a second exogenous or endogenous polynucleotide encoding a vacuolar antiporter. In some embodiments, the transgenic alga comprises a first exogenous or endogenous polynucleotide encoding a plasma membrane ATPase and a second exogenous or endogenous polynucleotide encoding a plasma membrane antiporter. In some embodiments, the transgenic alga comprises a first exogenous or endogenous polynucleotide encoding an H+-pyrophosphatase and second exogenous or endogenous polynucleotide encoding an antiporter. In some embodiments, the transgenic alga comprises a first exogenous or endogenous polynucleotide encoding a vacuolar H+-pyrophosphatase and a second exogenous or endogenous polynucleotide encoding a vacuolar antiporter. In some embodiments, the transgenic alga further comprises a third exogenous or endogenous polynucleotide encoding a vacuolar chloride channel protein. In some embodiments, the transgenic alga comprises an exogenous or endogenous polynucleotide encoding a BBC protein or a functional homolog thereof, SCSR protein or a functional homolog thereof, a chaperonin, or an antioxidant enzyme. In some embodiments, the antioxidant protein is glutathione peroxidase, ascorbate peroxidase, catalase, alternative oxidase, or superoxide dismutase.

In some embodiments, the transgenic alga comprises a first exogenous or endogenous polynucleotide encoding a transporter or a protein that regulates expression of a transporter and a second exogenous or endogenous polynucleotide encoding: a therapeutic protein, a nutritional protein, or an industrial enzyme; a protein that participates in or promotes the synthesis of at least one nutritional, therapeutic, commercial, or fuel product by the photosynthetic unicellular organism; or a protein that facilitates the isolation of at least one nutritional, therapeutic, commercial, or fuel product from the photosynthetic unicellular organism. In some embodiments, the second exogenous or endogenous polynucleotide encodes a biodegradative enzyme. In some embodiments, the second exogenous or endogenous polynucleotide encodes exo-β-glucanase, endo-β-glucanase, β-glucosidase, endoxylanase or lignase. In some embodiments, the second exogenous or endogenous polynucleotide encodes a flocculating moiety. In some embodiments, the second exogenous or endogenous polynucleotide encodes a botryococcene synthase, limonene synthase, 1,8 cineole synthase, α-pinene synthase, camphene synthase, (+)-sabinene synthase, myrcene synthase, abietadiene synthase, taxadiene synthase, farnesyl pyrophosphate synthase, amorphadiene synthase, (E)-α-bisabolene synthase, diapophytoene synthase, or diapophytoene desaturase.

In another aspect, the present disclosure provides a method for increasing salt tolerance of a eukaryotic microalga comprising introducing an exogenous or endogenous sequence into a photosynthetic unicellular organism, wherein the exogenous or endogenous sequence encodes an ion transporter or a protein that regulates the expression of a transporter, to produce a eukaryotic microalga having increased salt tolerance. The method further comprises plating the eukaryotic microalga on solid or semisolid selection media or inoculating the photosynthetic unicellular organism into a liquid selection media, wherein the selection media comprises a concentration of salt that does not permit growth of the organism not comprising the exogenous or endogenous sequence conferring salt resistance; and selecting at least one eukaryotic microalga comprising the exogenous or endogenous sequence conferring salt resistance by the viability of at least one eukaryotic microalga on or in the selection media. In some embodiments, the second exogenous or endogenous sequence is an ion transporter. In some embodiments, the transporter protein is an ATPase, an antiporter, or an H+ pyrophosphatase.

In another aspect, the present disclosure provides a method of selecting a transformant comprising an exogenous or endogenous polynucleotide sequence encoding a protein of interest, comprising: introducing a first polynucleotide encoding a protein of interest into an alga; introducing a second exogenous or endogenous polynucleotide into the alga, wherein the second exogenous or endogenous sequence confers salt tolerance; plating the alga on solid or semisolid selection media or inoculating the photosynthetic unicellular organism into liquid selection media, wherein the selection media comprises a concentration of salt that does not permit growth of the alga not comprising the exogenous or endogenous sequence conferring salt tolerance, and selecting at least one alga comprising the first exogenous or endogenous sequence by the viability of the at least one alga on or in the selection medium. In some embodiments, the first and the second exogenous or endogenous polynucleotides are on different nucleic acid molecules. In some embodiments, the first and the second exogenous or endogenous polynucleotides are on the same nucleic acid molecule. In some embodiments, the second exogenous or endogenous polynucleotide encodes a transporter, a protein that regulates the expression of a transporter, bbc protein or a functional homolog thereof, SCSR protein or a functional homolog thereof, a chaperonin, or an antioxidant enzyme. In some embodiments, the second exogenous or endogenous polynucleotide encodes an ion transporter. In some embodiments, the ion transporter is an ATPase, an antiporter, or a H+ pyrophosphatase. In some embodiments, the first polynucleotide encodes a therapeutic protein, a nutritional protein, or an industrial enzyme; a protein that participates in or promotes the synthesis of at least one nutritional, therapeutic, commercial, or fuel product by the photosynthetic unicellular organism; or a protein that facilitates the isolation of at least one nutritional, therapeutic, commercial, or fuel product from the photosynthetic unicellular organism. In some embodiments, the salt is a sodium salt. In some embodiments, the concentration of sodium in the selection media is at least 200 mM. In other embodiments, the salt is a lithium salt. In some embodiments, the concentration of lithium in the selection medium is at least 2 mM.

In yet another aspect, the present disclosure provides a method for producing one or more biomolecules, comprising: growing transgenic alga transformed with a polynucleotide encoding an ion transporter or protein that regulates the expression of an ion transporter, at a concentration of salt that inhibits the growth of non-transformed alga; and harvesting one or more biomolecules from the alga. In some embodiments, one or more biomolecules is a nutritional, therapeutic, commercial, or fuel product. In some embodiments, the method further comprises transforming the alga with an exogenous or endogenous polynucleotide encoding a therapeutic protein, a nutritional protein, or an industrial enzyme; a protein that participates in or promotes the synthesis of at least one nutritional, therapeutic, commercial, or fuel product by the photosynthetic unicellular organism; or a protein that facilitates the isolation of at least one nutritional, therapeutic, commercial, or fuel product from the photosynthetic unicellular organism. In some embodiments, the salt is a lithium salt. In some embodiments, the concentration of lithium in the selection media is at least 2 mM. In some embodiments, the salt is a sodium salt. In some embodiments, the concentration of sodium in the selection media is at least 200 mM.

### BRIEF DESCRIPTION OF THE DRAWINGS

These and other features, aspects, and advantages of the present disclosure will become better understood with regard to the following description, appended claims and accompanying figures where:

FIG. 1 shows a nuclear expression vector useful in the disclosed embodiments.

FIG. 2 shows growth of untransformed algae and algae transformed with SR8 in the presence of 250 mM added NaCl.

FIG. 3 shows growth curves of algae transformed with several SR genes in the presence of varying concentrations of added NaCl, as compared to untransformed algae.

FIG. 4 shows salt tolerant phenotypes of progeny from matings of untransformed algae with algae transformed with SR8.

FIG. 5 shows growth of untransformed algae and algae transformed with several SR genes in the presence of varying concentrations of added NaCl.

FIG. 6 shows salt tolerant phenotypes of progeny from matings of untransformed algae with algae transformed with SR1.

FIG. 7 shows salt tolerant phenotypes of progeny from matings of untransformed algae with algae transformed with SR2.

FIG. 8 shows salt tolerant phenotypes of progeny from matings of untransformed algae with algae transformed with SR3.

FIG. 9 shows PCR results for screening for the presence of the SR3 gene in transformed algae.

FIG. 10 shows PCR results for screening for the presence of the SR8 gene in transformed algae.

FIG. 11 shows growth curves of algae transformed with SR8 in the presence of varying concentrations of added NaCl, as compared to untransformed algae.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present disclosure.

As used in this specification and the appended claims, the singular forms “a”, “an” and “the” include plural reference unless the context clearly dictates otherwise.

Endogenous

An endogenous nucleic acid, nucleotide, polypeptide, or protein as described herein is defined in relationship to the host organism. An endogenous nucleic acid, nucleotide, polypeptide, or protein is one that naturally occurs in the host organism.

Exogenous

An exogenous nucleic acid, nucleotide, polypeptide, or protein as described herein is defined in relationship to the host organism. An exogenous nucleic acid, nucleotide, polypeptide, or protein is one that does not naturally occur in the host organism or is a different location in the host organism.

Salt Tolerance

A salt tolerant organism is able to grow in a saline environment that a wild-type or unmodified or untransformed organism of the same type cannot grow in. For example, a salt tolerant organism will be able to grow in a media containing a certain concentration of salt that its untransformed counterpart would not be able to grow in.

A salt tolerant organism is an organism that has been transformed with a nucleic acid that confers salt tolerance to the organism and the transformed organism is able to live and/or grow in an environment (for example, media) that has a salt concentration that an untransformed organism would not be able to live and/or grow in.

A protein can, for example, confer salt tolerance to an organism by reducing the effects of a stressful environment, such as salinity, on an organism. Such a gene or protein can be called a stress response gene or protein. One such exemplary protein is a glutathione peroxidase protein.

Disclosed herein is a vector comprising a polynucleotide capable of transforming a photosynthetic organism, comprising at least one nucleic acid sequence encoding a protein that when expressed in the photosynthetic organism, results in the photosynthetic organism becoming a salt tolerant photosynthetic organism as compared to a photosynthetic organism that is not transformed by the nucleic acid.

Disclosed herein is a vector comprising a polynucleotide capable of transforming a photosynthetic organism, comprising at least one nucleic acid sequence encoding a glutathione peroxidase (GPX) protein that when expressed in the photosynthetic organism, results in the photosynthetic organism becoming a salt tolerant photosynthetic organism as compared to a photosynthetic organism that is not transformed by the nucleic acid.

Disclosed herein is an isolated photosynthetic organism comprising an exogenous polynucleotide capable of transforming the photosynthetic organism, wherein the exogenous polynucleotide comprises at least one nucleic acid sequence encoding a protein that when expressed in the photosynthetic organism, results in the photosynthetic organism becoming a salt tolerant photosynthetic organism as compared to a photosynthetic organism that is not transformed by the nucleic acid.

Disclosed herein is an isolated photosynthetic organism comprising an exogenous polynucleotide capable of transforming the photosynthetic organism, wherein the exogenous polynucleotide comprises at least one nucleic acid sequence encoding a glutathione peroxidase (GPX) protein that when expressed in the photosynthetic organism, results in the photosynthetic organism becoming a salt tolerant photosynthetic organism as compared to a photosynthetic organism that is not transformed by the nucleic acid.

Disclosed herein is a method for increasing salt tolerance of a photosynthetic organism comprising, (a) transforming the photosynthetic organism with an exogenous nucleic acid sequence, wherein the nucleic acid sequence encodes a protein that when expressed in the photosynthetic organism, results in increased salt tolerance of the photosynthetic organism as compared to a photosynthetic organism that is not transformed by the nucleic acid.

Disclosed herein is a method for increasing salt tolerance of a photosynthetic organism comprising, (a) transforming the photosynthetic organism with an exogenous nucleic acid, wherein the nucleic acid sequence encodes a glutathione peroxidase (GPX) protein that when expressed in the photosynthetic organism, results in increased salt tolerance of the photosynthetic organism as compared to a photosynthetic organism that is not transformed by the nucleic acid.

Disclosed herein is a vector comprising a polynucleotide capable of transforming a photosynthetic organism, wherein the polynucleotide comprises at least one nucleic acid sequence encoding a protein comprising an amino acid sequence of SEQ ID NO: 56, wherein when the protein is expressed in the photosynthetic organism, the photosynthetic organism becomes a salt tolerant photosynthetic organism as compared to a photosynthetic organism that is not transformed by the nucleic acid.

Disclosed herein is an isolated photosynthetic organism comprising an exogenous polynucleotide capable of transforming the photosynthetic organism, wherein the exogenous polynucleotide comprises at least one nucleic acid sequence encoding a protein comprising an amino acid sequence of SEQ ID NO: 56, wherein when the protein is expressed in the photosynthetic organism, the photosynthetic organism becomes a salt tolerant photosynthetic organism as compared to a photosynthetic organism that is not transformed by the nucleic acid.

Disclosed herein is a method for increasing salt tolerance of a photosynthetic organism comprising, (a) transforming the photosynthetic organism with an exogenous polynucleotide sequence comprising a nucleic acid sequence, wherein the nucleic acid sequence encodes a protein comprising an amino acid sequence of SEQ ID NO: 56, wherein expression of the protein in the photosynthetic organism results in increased salt tolerance of the photosynthetic organism as compared to a photosynthetic organism that is not transformed by the nucleic acid.

Disclosed herein are compositions and methods relating to engineering salt tolerance in photosynthetic microorganisms, for example, microalgae Chlamydomonas reinhardtii. In one aspect, the present disclosure provides a method for increasing salt tolerance of a eukaryotic microalga comprising introducing an exogenous sequence into a photosynthetic unicellular organism, wherein the exogenous sequence encodes an ion transporter or a protein that regulates the expression of an ion transporter to produce a eukaryotic microalga having increased salt tolerance. In another aspect, the present disclosure provides a method of selecting a transformant comprising an exogenous polynucleotide sequence encoding a protein of interest, comprising: introducing a first polynucleotide encoding a protein of interest into an alga, introducing a second polynucleotide into the alga, wherein the second polynucleotide sequence confers salt tolerance; plating the alga on solid or semisolid selection media or inoculating the photosynthetic unicellular organism into liquid selection media, wherein the selection media comprises a concentration of salt that does not permit growth of the alga not comprising the polynucleotide sequence conferring salt tolerance; and selecting at least one alga comprising the first polynucleotide sequence by the viability of the at least one alga on or in the selection medium. In yet another aspect, the present disclosure describes a method for producing one or more biomolecules, comprising: growing transgenic alga transformed with a polynucleotide encoding an ion transporter or protein that regulates the expression of an ion transporter, at a concentration of salt that inhibits the growth of non-transformed alga of the same species; and harvesting one or more biomolecules from the alga.

Plant species vary in how well they tolerate salt. Some plants will tolerate high levels of salinity while others can tolerate little or no salinity. The relative growth of plants in the presence of salinity is termed their salt tolerance. Salt tolerance is the ability of a plant or plant cell to display an improved response to an increase in extracellular and/or intracellular concentration of salt including, but not limited to, Na+, Li+ and K+, as compared to a wild-type plant. Increased salt tolerance may be manifested by phenotypic characteristics including longer life span, apparent normal growth and function of the plant, and/or a decreased level of necrosis, when subjected to an increase in salt concentration, as compared to a wild-type plant. Salt tolerance is measured by methods known in the art such as those described in Inan et al. (July 2004) Plant Physiol. 135:1718, including without limitation, NaCl shock exposure or a gradual increase in NaCl concentration.

In one aspect, the present disclosure provides an engineered photosynthetic microorganism, such as a unicellular transgenic alga, with an increased salt tolerance. In some embodiments, the present disclosure provides a transgenic organism comprising an exogenous polynucleotide encoding an ion transporter, such as an ATPase ion transporter. The present disclosure also provides a transgenic alga comprising an exogenous polynucleotide encoding a transporter or a protein that regulates expression of a transporter. The present disclosure also provides a transgenic organism comprising an exogenous polynucleotide encoding a vacuolar transporter. Also disclosed in the present disclosure is a transgenic organism comprising an exogenous polynucleotide encoding a transporter or a protein that regulates expression of a transporter, wherein the transporter does not transport a catabolizable carbon source, further wherein the polynucleotide is codon biased for the nuclear genome of the organism. In some embodiments, the present disclosure provides a transgenic organism comprising an exogenous polynucleotide encoding an ion transporter or a protein that regulates expression of an ion transporter, wherein the polynucleotide is codon biased for the nuclear genome of the organism. The present disclosure also provides a transgenic organism comprising two or more exogenous polynucleotides, wherein at least one of the exogenous polynucleotides encodes a transporter or a protein that regulates expression of a transporter, wherein the transporter does not transport a catabolizable carbon source. In some embodiments, the present disclosure provides a transgenic organism comprising two or more exogenous polynucleotides, wherein at least one of the exogenous polynucleotides encodes an ion transporter or a protein that regulates expression of an ion transporter. The present disclosure also describes a transgenic eukaryotic organism, for example, a unicellular alga comprising an exogenous polynucleotide encoding a transporter or a protein that regulates expression of a transporter, wherein the transporter does not transport a catabolizable carbon source. In some embodiments, the present disclosure provides a transgenic eukaryotic unicellular alga comprising an exogenous polynucleotide encoding an ion transporter or a protein that regulates expression of an ion transporter.

A transgenic algal cell of the present disclosure has increased salt tolerance with respect to an algal cell that does not comprise the exogenous polynucleotide encoding the transporter. In some embodiments, the salt tolerance is at least twice, at least three times, or at least four times that of a wild type alga. The salt tolerance can be at least 0.5, at least 1.0, at least 1.5, at least 2.0, at least 2.5, at least 3.0, at least 3.5, at least 4.0, at least 5.0, or more than about 5 fold higher than that of a wild type alga.

The salt used in the present disclosure can be, for example, a sodium (Na+) salt, a lithium (Li+) salt, or a potassium (K+) salt. The concentration of NaCl added to the selection media for the transgenic algae of the present disclosure can be, for example, at least 25 mM. The concentration of Li+ added to the selection media for the transgenic algae of the present disclosure can be, for example, at least 2 mM.

The salt concentration depends on the media composition that is used for the experiment. One of skill in the art would be able to determine an appropriate range of salt to use. For example, for NaCl, if the algae (C. reinhardtii) are grown in media (TAP), a range of about 250 to about 300 mM NaCl can be added to the media to select for strains with a higher salt tolerance than a wild type algae. A wild type algae may die, for example, at around 150-200 mM added NaCl. One of skill in the art would be able to select a salt and determine the range of concentrations of the salt without undue experimentation.

Transporters

In one aspect, the present disclosure provides an expression vector comprising a polynucleotide encoding a transporter or a protein that regulates the expression of a transporter, wherein the polynucleotide is codon biased for the nuclear genome of the host, wherein the transporter does not transport a reduced carbon source. In another aspect, the present disclosure provides a transgenic eukaryotic unicellular organism comprising an exogenous polynucleotide encoding an ion transporter or a protein that regulates expression of an ion transporter. The ion transporter can be an ATPase. In another aspect, the present disclosure provides a method for increasing salt tolerance of a eukaryotic organism comprising introducing an exogenous sequence into a photosynthetic unicellular organism, wherein the exogenous sequence encodes a transporter or a protein that regulates the expression of a transporter, to produce a eukaryotic organism having increased salt tolerance.

A transporter can transport, for example, Li+, Na+, or K+, across a membrane. In some embodiments, the transporter is an ATPase. The ATPase may be a Na+ ATPase, a K+ ATPase, or a Li+ ATPase. The transporter can also be a P-type ATPase. The P-type ATPase can be ENA1 or a functional homolog thereof.

ATPases are a class of enzymes that catalyze the decomposition of adenosine triphosphate (ATP) into adenosine diphosphate (ADP) and a free phosphate ion. Transmembrane ATPases import many of the metabolites necessary for cell metabolism and export toxins, wastes, and solutes that can hinder cellular processes. One example is the sodium-potassium exchanger (or Na⁺/K⁺ ATPase), which establishes the ionic concentration balance that maintains a cell's potential. Na⁺/K⁺-ATPase is an enzyme located in the plasma membrane (specifically an electrogenic transmembrane ATPase). It is found in humans and animals. Active transport is responsible for the well-established observation that cells contain relatively high concentrations of potassium ions but low concentrations of sodium ions. The mechanism responsible for this is the sodium-potassium pump which moves these two ions in opposite directions across the plasma membrane. The Na⁺/K⁺-ATPase helps maintain resting potential, avail transport, and regulate cellular volume. It also functions as a signal transducer/integrator to regulate the MAPK pathway, ROS (Reactive Oxygen Species), as well as intracellular calcium levels. Na+ pumping ATPases are a class of membrane bound proteins that actively pump Na+ ions out of cells. They belong to the P-type superfamily of ATP-driven pumps, and in particular to a separate phylogenetic group, the type IID ATPases.

In some embodiments, the ATPase is a P-type ATPase. The P-type ATPase can be a yeast, plant, or algal P-type ATPase, for example. P-ATPases (E1E2-ATPases) are found in bacteria, fungi and in eukaryotic plasma membranes and organelles. P-ATPases function to transport a variety of different compounds, including ions and phospholipids, across a membrane using ATP hydrolysis for energy. There are many different classes of P-ATPases, each of which transports a specific type of ion: H+, Na+, K+, Mg2+, Ca2+, Ag+, Ag2+, Zn2+, Co2+, Pb2+, Ni2+, Cd2+, Cu+ and Cu2+. P-ATPases can be composed of one or two polypeptides, and can usually assume two main conformations called E1 and E2.

In some embodiments, the P-type ATPase is ENA1 or a functional homolog thereof. In Saccharomyces cerevisiae, the ENA1 gene plays an important role in salt tolerance. This gene encodes a P-type Na⁺-ATPase that is an important element in the efflux of Na⁺ and Li⁺. An ena1 mutant can be highly sensitive even to low concentrations of Na⁺ or Li⁺ (Garciadeblás, B., et al. 1993. Mol. Gen. Genet. 236:363-368). The ENA1 gene is barely expressed under standard growth conditions, but it is strongly induced by exposure to high salt concentrations and to an alkaline pH. This transcriptional response of ENA1 is based on a complex regulation of its promoter (Márquez, J. A., and R. Serrano. 1996. FEBS Lett. 382:89-92). Expression of ENA1 is repressed by the presence of glucose in the medium, through a mechanism that involves the general repressor complex Mig1-Ssn6-Tup1 (Proft, M., and R. Serrano. 1999. Mol. Cell. Biol. 19:537-546). Saline induction is mediated by two pathways: the Hog1 mitogen-activated protein kinase pathway and the calcineurin pathway. The Hog1 pathway responds to increased osmolarity and acts through the Sko1 transcriptional inhibitor, which binds to a cyclic AMP response element present in the ENA1 promoter.

Antiporters

Antiporters are discussed in Law, C. J., et al. Annu Rev Microbiol. (2008) 62: 289-305. The major facilitator superfamily (MFS) represents the largest group of secondary active membrane transporters, and its members transport a diverse range of substrates. Recent work shows that MFS antiporters, and perhaps all members of the MFS, share the same three-dimensional structure, consisting of two domains that surround a substrate translocation pore. The advent of crystal structures of three MFS antiporters sheds light on their fundamental mechanism; they operate via a single binding site, alternating-access mechanism that involves a rocker-switch type movement of the two halves of the protein. In the sn-glycerol-3-phosphate transporter (GlpT) from Escherichia coli, the substrate-binding site is formed by several charged residues and a histidine that can be protonated. Salt-bridge formation and breakage are involved in the conformational changes of the protein during transport.

In some embodiments, the transporter is an antiporter. Antiporters (also called exchangers or counter-transporters) are integral membrane proteins which are involved in secondary active transport of two or more different molecules or ions (i.e. solutes) across a phospholipid membrane, such as the plasma membrane, in opposite directions. In secondary active transport, one species of solute moves along its electrochemical gradient, allowing a different species of solute to move against its own electrochemical gradient. This movement is in contrast to primary active transport, in which all solutes are moved against their concentration gradients, fueled by ATP. Transport may involve one or more of each type of solute. For example, the Na⁺/Ca²⁺ exchanger, used by many cells to remove cytoplasmic calcium, exchanges one calcium ion for three sodium ions. Thus, in some embodiments, an organism is transformed with one or more transporters such as a Na+ antiporter, a NHX protein, a SOS1 antiporter, a CAX antiporter, an Nha protein, a Nap protein, or a functional homolog of any of the above.

As used herein, a “homolog” refers to a protein that has similar action, structure, antigenic, and/or immunogenic response as the protein of interest. It is not intended that a homolog and a protein of interest be necessarily related evolutionarily. Thus, it is intended that the term encompass the same functional protein obtained from different species. In some embodiments, it is desirable to identify a homolog that has a tertiary and/or primary structure similar to the protein of interest, as replacement of the epitope in the protein of interest with an analogous segment from the homolog will reduce the disruptiveness of the change. Thus, in some embodiments, closely homologous proteins provide the most desirable sources of epitope substitutions. In addition, it may be advantageous to look at the human analogs of a given protein. The homology between a transporter and its functional homolog can be greater than about 10%, greater than about 200%, greater than about 30%, greater than about 40%, greater than about 50%, greater than about 60%, greater than about 70%, greater than about 80%, greater than about 90%, greater than about 95%, greater than about 98%, or greater than about 99% sequence

GPX Proteins

Glutathione peroxidase is the general name of an enzyme family with peroxidase activity whose main biological role is to protect the organism from oxidative damage. The biochemical function of glutathione peroxidase is to reduce lipid hydroperoxides to their corresponding alcohols and to reduce free hydrogen peroxide to water.

There are several isozymes encoded by different genes, which vary in cellular location and substrate specificity. Glutathione peroxidase 1 (GPx1) is the most abundant version, found in the cytoplasm of nearly all mammalian tissues, whose preferred substrate is hydrogen peroxide. Glutathione peroxidase 4 (GPx4) has a high preference for lipid hydroperoxides; it is expressed in nearly every mammalian cell, though at much lower levels. Glutathione peroxidase 2 is an intestinal and extracellular enzyme, while glutathione peroxidase 3 is extracellular, especially abundant in plasma. So far, eight different isoforms of glutathione peroxidase (GPx1-8) have been identified in humans.

Glutathione peroxidase is an enzyme which catalyzes a reaction of two moles of glutathione and one mole of hydrogen peroxide to form two moles of glutathione-oxide and two moles of water, and is found in mammalian tissues and organs such as liver, kidney, heart, lung, red blood cells and blood plasma (Flohe, L. et al., FEBS Letters, 32: 132-134 (1973)). It plays an important role in the treatment of biological peroxide by catalyzing the reduction by two electrons of lipid-peroxide with glutathione. Glutathione peroxidase is a protein containing selenium which has the amino acid selenocystein (Sec) in its active center. According to a study on a cloned mouse glutathione peroxidase gene, the opal codon, TGA, of the corresponding ribonucleic acid sequence (RNA), which is in general a termination codon, in this enzyme codes for selenocystein (Sec) (EMBO J., Vol. 5, No 6, pp. 1221-1227 (1986)).

An example reaction that glutathione peroxidase catalyzes is: 2GSH+H₂O₂→GS-SG+2H₂O, where GSH represents reduced monomeric glutathione, and GS-SG represents glutathione disulfide. Glutathione reductase then reduces the oxidized glutathione to complete the cycle: GS-SG+NADPH+H⁺→2 GSH+NADP⁺.

Mammalian GPx1, GPx2, GPx3, and GPx4 have been shown to be selenium-containing enzymes, whereas GPx6 is a selenoprotein in humans with cysteine-containing homologues in rodents. GPx1, GPx2, and GPx3 are homotetrameric proteins, whereas GPx4 has a monomeric structure. As the integrity of the cellular and subcellular membranes depends heavily on glutathione peroxidase, the antioxidative protective system of glutathione peroxidase itself depends heavily on the presence of selenium.

A human-type glutathione peroxide (sometimes designated h-GSHPx) has been separated from erythrocyte and blood plasma, and has been known to be a homotetramar, in which the molecular weight of the four erythrocyte type subunits is each 20,600 and that of the blood plasma type subunits is each 21,500 (Archives of Biochemistry and Biophysics, Vol. 256, (2): 677-686 (1987); and The Journal of Biological Chemistry, Vol. 262 (36): pp. 17398-17403 (1987)).

H-GSHPx's derived from erythrocytes, liver and kidney are believed to be identical due to their strong immunological cross reactivity and similar subunit molecular weight of approximately 20,600. The h-GSHPx gene is quite homologous to the mouse GSHPx gene, however the mouse h-GSHPx gene product shares little immunological similarity with h-GSHPx derived from blood plasma.

These h-GSHPx's were cloned from a c-DNA library of m-RNA isolated from liver and kidney cells, and their gene structure has been determined (Nucleic Acids Research, Vol. 15, No. 13, pp. 5484 (1987); and Nucleic Acids Research, Vol. 15, No. 17, pp. 7178 (1987)). h-GSHPx protein of blood plasma has been isolated.

Glutathione peroxidase has been described by Muller, F. L., et al. (2007) Free Radic. Biol. Med. 43 (4): 477-503; Ran, Q., et al. (2007) J. Gerontol. A Biol. Sci. Med. Sci. 62 (9): 932-42; and Mills, G. C. (1957), J. Biol. Chem. 229 (1): 189-97.

Several genes encoding putative glutathione peroxidase have been isolated from a variety of plants, all of which show the highest homology to the phospholipid hydroperoxide isoform. Several observations suggest that the proteins are involved in biotic and abiotic stress responses. Previous studies on the regulation of gpx1, the Citrus sinensis gene encoding phospholipid hydroperoxide isoform, led to the conclusion that salt-induced expression of the gpx1 transcript and its encoded protein is mediated by oxidative stress. Avsian-Kretchmer, O., et al., Plant Physiology 135:1685-1696 (2004) describes the induction of gpx1 promoter:uid4 fusions in stable transformants of tobacco (Nicotiana tabacum) cultured cells and plants. In these studies, it is shown that the induction of gpx1 by salt and oxidative stress occurs at the transcriptional level. Gpx1 promoter analysis confirmed previous assumptions that the salt signal is transduced via oxidative stress. The induction of the fusion construct was used to achieve better insight into, and to monitor salt-induced oxidative stress. The gpx1 promoter responded preferentially to oxidative stress in the form of hydrogen peroxide, rather than to superoxide-generating agents. Antioxidants abolished the salt-induced expression of the gpx1 promoter, but were unable to eliminate the induction by H₂O₂. The commonly employed NADPH-oxidase inhibitor diphenyleneiodonium chloride and catalase inhibited the H₂O₂-induced expression of the gpx1 promoter, but did not affect its induction by salt. These results indicate that salt induces oxidative stress in the form of H₂O₂, its production occurs in the intracellular space, and its signal transduction pathway activating the gpx1 promoter is different from the pathway induced by extracellular H₂O₂.

Detrimental effects of salinity on plants are known to be partially alleviated by external Ca²⁺. Gueta-Dahan, Y., et al., Planta (2008) 228(5):725-734 demonstrated that in citrus cells, phospholipid hydroperoxide glutathione peroxidase (GPX1) is induced by salt and its activation can be monitored by a pGPX1::GUS fusion in transformed tobacco cells. Gueta-Dahan, Y., et al. (Planta (2008) 228(5):725-734) further characterized the induction of GPX1 by additional treatments, which are known to affect Ca²⁺ transport. Omission of Ca²⁺ changed the pattern of the transient salt-induced expression of GPX1 and chelation of Ca²⁺ by EGTA, or treatment with caffeine, abolished the salt-induced GPX1 transcript. On the other hand. La³⁺ was found to be as potent as NaCl in inducing GPX1 transcription and the combined effect of La³⁺ and NaCl seemed to be additive. Pharmacological perturbation of either external or internal Ca²⁺ pools by La³⁺, EGTA, caffeine, Ca²⁺ channel blockers, or a Ca²⁺-ATPase inhibitor rendered the imposed salt stress more severe. Except for La³⁺, all these Ca²⁺ effectors had no effect on their own. In addition, the fluidizer benzyl alcohol dramatically increased the NaCl-induced GPX1 transcription. Taken together, these results show that: 1) the mode of action of La³⁺ on GPX1 expression differs from its established role as a Ca²⁺ channel blocker, 2) membrane integrity has an important role in the perception of salt stress, and 3) internal stores of Ca²⁺ are involved in activating GPX1 expression in response to salt stress.

The GPX gene is also discussed in Miyasaka et al. (2000) World Journal of Microbiology and Biotechnology, 16:23-29.

Voltage Gated Ion Channels

Voltage-gated ion channels are a class of transmembrane ion channels that are activated by changes in electrical potential difference near the channel; these types of ion channels are critical in neurons, but are common in many types of cells.

Voltage-gated ion channels have a crucial role in excitable neuronal and muscle tissues, allowing a rapid and coordinated depolarisation in response to triggering voltage change. Found along the axon and at the synapse, voltage-gated ion channels directionally propagate electrical signals.

Voltage-gated ion channels generally are composed of several subunits arranged in such a way that there is a central pore through which ions can travel down their electrochemical gradients. The channels tend to be ion-specific, although similarly sized and charged ions may sometimes travel through them.

Examples of voltage-gated ion channels are sodium and potassium voltage-gated channels found in nerve and muscle, and the voltage-gated calcium channels that play a role in neurotransmitter release in pre-synaptic nerve endings.

From crystallographic structural studies of a potassium channel, assuming that this structure remains intact in the corresponding plasma membrane, it is possible to surmise that when a potential difference is introduced over the membrane, the associated electromagnetic field induces a conformational change in the potassium channel. The conformational change distorts the shape of the channel proteins sufficiently such that the cavity, or channel, opens to admit ion influx or efflux to occur across the membrane, down its electrochemical gradient. This subsequently generates an electrical current sufficient to depolarise the cell membrane.

Voltage-gated sodium channels and calcium channels are made up of a single polypeptide with four homologous domains. Each domain contains 6 membrane spanning alpha helices. One of these helices, S4, is the voltage sensing helix. It has many positive charges such that a high positive charge outside the cell repels the helix-inducing a conformational change such that ions may flow through the channel. Potassium channels function in a similar way, with the exception that they are composed of four separate polypeptide chains, each comprising one domain.

The voltage-sensitive protein domain of these channels (the “voltage sensor”) generally contains a region composed of S3b and S4 helices, known as the “paddle” due to its shape, which appears to be a conserved sequence, interchangeable across a wide variety of cells and species. Genetic engineering of the paddle region from a species of volcano-dwelling archaebacteria into rat brain potassium channels results in a fully functional ion channel, as long as the whole intact paddle is replaced.

Voltage-gated ion channels are described in Alabi, A. A., et al., Nature (2007) 450 (7168):370-5; and Long, S. B., et al., Nature (2007) 450 (7168):376-382.

An exemplary, predicted voltage dependent potassium channel that can be used in the current embodiments, is shown in SEQ ID NOs 57-62.

NHX Proteins

In some embodiments, the transporter is an antiporter. The antiporter can be NHX1 or a functional homolog thereof. The NHX protein is a sodium (Na+) antiporter and as an active Na+ pump, the NHX protein is involved in extruding Na+ ions from the cytoplasm into the vacuole of a cell. The vacuolar localized NHX1, belonging to the NHX family of proteins, is found in a wide variety of organisms including humans. In plants and fungi, NHX1 mediates the sodium sequestration in the vacuole under salt stress conditions. This is one of the mechanisms used by a plant to protect the cells against high salinity in the soil and in the water.

Na+-H+ exchangers are a family of integral membrane phosphoglycoproteins that play an important role in the regulation of intracellular pH and sodium homeostasis by mediating the counter transport of extracellular sodium and intracellular protons (for example, as described in Wakabayashi, S, and Shigekawa, M., Physiol. Rev. (1997) 77:51-74; and Orlowski, J. and Grinstein, S., J. Biol. Chem. (1997) 272:22373-22376). NHX genes have been isolated from a number of plant species, such as Arabidopsis (for example, as described in Gaxiola et al. (1999) PNAS 96(4), 1485), rice (OsNHX (for example, as described in Fukuda et al., Biochim Biophys Acta. (1999) Jul. 7; 1446(1-2):149-55), and from Atriplex (AgNHX, JP2000157287).

Transgenic plants over expressing the Arabidopsis gene AtNHX have been shown to have increased tolerance to high salinity (for example, about 200 to about 400 mM NaCl) in growth media. Examples of salt-tolerant Arabidopsis plants, tomato plants and Brassica have been described (for example, as described in Apse et al., Science (1999) Aug. 20; 285 (5431): 1256-8; Apse M P, Blumwald E., Curr Opin Biotechnol. (2002) April; 13(2):146-50; Zhang and Blumwald, Nat. Biotechnol. (2001) August; 19(8):7658; and Zhang et al., Proc Natl Acad Sci USA (2001) Oct. 23; 98(22): 12832-6). For example, transgenic Brassica napus plants over expressing AfNHX were able to grow, flower, and produce seeds in the presence of 200 mM sodium chloride. Although transgenic plants grown in high salinity accumulated sodium to up to 6% of their dry weight, growth of the these plants was only marginally affected by the high salt concentration. Moreover, seed yield and the seed oil quality were not affected by the high salinity of the soil. Furthermore, salt tolerant monocots were generated by transformation into plants of an NHX gene. Ohta et al. (FEBS Lett. (2002) Dec. 18; 532(3):279-82) engineered a salt-sensitive rice cultivar (Oryza sativa Kinuhikari) to express a vacuolar-type Na+/H+ antiporter gene from the halophytic plant, Atriplex gmelini (AgNHX). The activity of the vacuolar-type Na+/H+ antiporter in the transgenic rice plants was eight-fold higher than that of wild-type rice plants. Salt tolerance assays followed by non-salt stress treatments showed that the transgenic plants over expressing AgNHX could survive under conditions of 300 mM NaCl for 3 days whilst the wild-type rice plants could not. This indicates that over expression of the Na+/H+ antiporter gene in rice plants significantly improves their salt tolerance. After salt-stress treatments, the surviving transgenic rice plants were transferred to soil conditions without salt stress and were grown in a greenhouse. Although the number of tillers was reduced compared to untreated transgenic rice plants, the transgenic rice plants grew until the flowering stage and set seeds after 3.5 months, demonstrating that the salt shock did not completely damage the fertility of the transgenic rice plants. All these transgenic plants showed better survival capacity when grown on high salinity media and showed “wild-type phenotypes” on the green biomass level and on the level of flowering and seed-production, whist the non-transgenic plants were suffering from salt toxicity. In tomato plants, the fruits of transgenic plants were smaller than the fruits of wild-type non-salt stressed plants. In summary, several reports have established a role for NHX genes in salt tolerance.

Evaluation of the Chlamydomonas genome with the yeast and Arabidopsis NHX1 gene did not clearly reveal the presence of any NHX1 homologs. Therefore, engineering of algae, for example, Chlamydomonas reinhardtii, to over express a plant or yeast NHX1 gene may confer enhanced salt tolerance to C. reinhardtii.

In one aspect, the present disclosure provides an expression vector comprising a polynucleotide encoding a transporter or a protein that regulates the expression of a transporter, wherein the polynucleotide is codon biased for the nuclear genome of an algal host, wherein the transporter does not transport a reduced carbon source. In another aspect, the present disclosure describes an expression vector comprising a polynucleotide encoding a transporter or a protein that regulates the expression of a transporter, operably linked to an exogenous promoter that functions in an algal cell, wherein the transporter does not transport a reduced carbon source. The disclosure also describes an expression vector comprising a polynucleotide encoding a non-algal transporter or a non-algal protein that regulates the expression of a transporter, operably linked to an algal regulatory sequence, wherein the transporter does not transport a reduced carbon source. In some embodiments, the transporter is an ion transporter. The ion transporter can be an antiporter. The antiporter can be NHX1 or a functional homolog thereof.

The sequence of SEQ ID NO 1 has previously been deposited in the GenBank under the accession number AB021878 and the corresponding protein, SEQ ID NO 2, has been deposited in GenBank under accession number BAA83337.

The term “essentially similar to” also includes a complement of the sequences of SEQ ID NO: 1 or SEQ ID NO: 2: RNA, DNA, a cDNA or a genomic DNA corresponding to the sequences of SEQ ID NO: 1 or SEQ ID NO: 2; a variant of the gene or protein due to the degeneracy of the genetic code: allelic variant of the gene or protein; and different splice variants of the gene or protein and variants that are interrupted by one or more intervening sequences. The term “essentially similar to” also includes family members or homologues, orthologues and paralogues of the gene or protein represented by the sequence of SEQ ID NO: 1 or SEQ ID NO: 2. Moreover, the conservation of NHX genes among diverse prokaryotic and eukaryotic species also allows the use of non-plant NHX genes for the methods of the present disclosure, such as NHX genes/proteins from yeast, fungi, molds, algae, plants, insects, animals, and human, for example.

It should be clear that the applicability of the disclosure is not limited to use of a nucleic acid represented by SEQ ID NO 1 nor to the nucleic acid sequence encoding an amino acid sequence of SEQ ID NO 2, but that other nucleic acid sequences encoding homologues, derivatives or active fragments of SEQ ID NO 1, or other amino acid sequences encoding homologues, derivatives or active fragments of SEQ ID NO 2, may be useful in the methods of the present disclosure. Nucleic acids suitable for use in the methods of the disclosure include those encoding NHX proteins according to the aforementioned definition, i.e. having: (i) the following consensus sequence: FFXXLLPPII; and (ii) having at least about 20%, about 25%, about 30%, about 35%, about 40%, about 45%, about 50%, about 55%, about 60%, about 65%, about 70%, about 75%, about 80%, about 85%, about 90%, about 95% or more sequence identity to the sequence represented by SEQ ID NO: 2; and/or (iii) having Na+/H+ activity.

Examples of NHX proteins include but are not limited to: AtNHX1 (AF106324: SEQ ID NO: 44 encoded by the sequence of SEQ ID NO: 3), AtNHX2 (AG009465), AtNHX3 (AC011623), AtNHX4 (AB015479), AtNHX5 (AC005287) and AtNHX6 (AC010793) all from Arabidopsis and described in Yokoi et al. (2002) “Differential expression and function of Arabidopsis thaliana NHX Na/H antiporters in the salt stress response” The Plant Journal, Volume 30, Issue 5, Pages 529-539.

SOS Pathway

In some embodiments, the antiporter is SOS1 or a functional homolog thereof. Another set of genes involved in salt tolerance are the SOS genes. Salt and drought stress signal transduction consists of ionic and osmotic homeostasis signaling pathways, detoxification (i.e., damage control and repair) response pathways, and pathways for growth regulation. The ionic aspect of salt stress is signaled via the SOS pathway where a calcium-responsive SOS3-SOS2 protein kinase complex controls the expression and activity of ion transporters such as SOS1. Osmotic stress activates several protein kinases including mitogen-activated kinases, which may mediate osmotic homeostasis and/or detoxification responses. A number of phospholipid systems are activated by osmotic stress, generating a diverse array of messenger molecules, some of which may function upstream of the osmotic stress-activated protein kinases. Abscisic acid biosynthesis is regulated by osmotic stress at multiple steps. Both ABA-dependent and -independent osmotic stress signaling first modify constitutively expressed transcription factors, leading to the expression of early response transcriptional activators, which then activate downstream stress tolerance effector genes (Zhu, Annu Rev Plant Biol. (2002) 53:247-73).

A regulatory pathway for ion homeostasis and salt tolerance was identified in A. thaliana (Zhu, 2000 and Zhu, Annu Rev Plant Biol. (2002) 53:247-73). Salt stress is known to elicit a rapid increase in the free calcium concentration in the cytoplasm (Knight. H., et al. (1997) Calcium signaling in Arabidopsis thaliana responding to drought and salinity, Plant J. 12: 1067-1078). It is proposed that SOS3, a myristoylated calcium binding protein, senses this calcium signal (Liu, J. and Zhu, J.-K. (1998) Science, 280: 1943-1945; and Ishitani, M., et al. (2000) SOS3 function in plant salt tolerance requires N-myristoylation and calcium-binding, Plant Cell, Vol. 12, 1667-1678). SOS3 physically interacts with the protein kinase SOS2 and activates the substrate phosphorylation activity of SOS2 in a calcium-dependent manner (Halfter, U., et al. (2000) The Arabidopsis SOS2 protein kinase physically interacts with and is activated by the calcium-binding protein SOS3, Proc Natl Acad Sci USA 97: 3735-3740; and Liu, J., et al. (2000) The Arabidopsis thaliana SOS2 gene encodes a protein kinase that is required for salt tolerance, Proc Natl Acad Sci USA 97: 3730-3734). SOS3 also recruits SOS2 to the plasma membrane, where the SOS3-OS2 protein kinase complex phosphorylates SOS1 to stimulate its Na+/H+ antiport activity (Qui, Q., et al. (2002) Proc Natl Acad Sci USA 99: 8436-8441; and Quintero, F. J., et al., Proc Natl Acad Sci USA (2002) 99(13): 9061-9066). Loss-of-function mutations in SOS3, SOS2, or SOS1 cause hypersensitivity to Na+ (for example, as described in Zhu, J.-K., et al. (1998) Genetic analysis of salt tolerance in Arabidopsis thaliana: evidence of a critical role for potassium nutrition, Plant Cell, 10: 1181-1192; Liu, J. and Zhu, J.-K. (1998) A calcium sensor homolog required for plant salt tolerance, Science, 280: 1943-1945; and Zhu, J.-K. (2000) Genetic Analysis of Plant Salt Tolerance Using Arabidopsis, Plant Physiology. November 2000, Vol. 124, pp. 941-948: Knight, H., et al. (1997) Calcium signaling in Arabidopsis thaliana responding to drought and salinity, Plant J. 12: 1067-1078; Ishitani, M., et al. (2000) SOS3 function in plant salt tolerance requires N-myristoylation and calcium-binding, Plant Cell, Vol. 12, 1667-1678; Halfter, U., et al. (2000) The Arabidopsis SOS2 protein kinase physically interacts with and is activated by the calcium-binding protein SOS3, Proc Natl Acad Sci USA 97: 3735-3740; Liu, J., et al. (2000) The Arabidopsis thaliana SOS2 gene encodes a protein kinase that is required for salt tolerance, Proc Natl Acad Sci USA 97: 3730-3734; Qui, Q., et al. (2002) Regulation of SOS1, a plasma membrane Na+/H+ exchanger in Arabidopsis thaliana, by SOS2 and SOS3, Proc Natl Acad Sci USA 99: 8436-8441; and Quintero, F. J., et al., Proc Natl Acad Sci USA (2002) 99(13): 9061-9066).

SOS2 has a highly conserved N-terminal catalytic domain similar to that of Saccharomyces cerevisiae SNF1 and animal AMPK (Liu, J., et al. (2000) The Arabidopsis thaliana SOS2 gene encodes a protein kinase that is required for salt tolerance, Proc Natl Acad Sci USA 97: 3730-3734). Within the SOS2 protein, the N-terminal catalytic region interacts with the C-terminal regulatory domain (Guo, Y., et al. (2001) Plant Cell 13, 1383-1400). SOS3 interacts with the FISL motif in the C-terminal region of SOS2 (Guo, Y., et al. (2001) Plant Cell 13, 1383-1400), which serves as an auto-inhibitory domain. A constitutively active SOS2 kinase, T/DSOS2, can be engineered by a Thr₁₆₈-to-Asp change (to mimic phosphorylation by an upstream kinase) in the putative activation loop. The kinase activity of T/DSOS2 is independent of SOS3 and calcium (Guo, Y., et al. (2001) Plant Cell 13, 1383-1400). Removing the FISL motif (SOS2DF) or the entire C-terminal regulatory domain (SOS2/308) may result in constitutively active forms of SOS2 (Guo, Y., et al. (2001) Plant Cell 13, 1383-1400; and Qui, Q., et al. (2002) Proc Natl Acad Sci USA 99: 8436-8441). The activation loop mutation and the autoinhibitory domain deletions have a synergistic effect on the kinase activity of SOS2, and superactive SOS2 kinases T/DSOS2/308 or T/DSOS2/DF can be created when the two changes are combined (Guo, Y., et al. (2001) Plant Cell 13, 1383-1400; and Qui, Q., et al. (2002) Proc Natl Acad Sci USA 99: 8436-8441). It has been shown that T/DSOS2/DF could activate the transport activity of SOS1 in vitro, whereas the wild-type SOS2 protein could not (for example, as described in Guo, Y., et al. (2001) Plant Cell 13, 1383-1400).

In some embodiments, the polynucleotide of the present disclosure encodes at least one component of the SOS pathway, for example, SOS2 and/or SOS3. The polynucleotide may encode a wild type or a mutant SOS2 or SOS3. Mutations can include, for example, one or more substitution, addition, or deletion of a nucleic acid or amino acid. Exemplary sequences of the present disclosure correspond to mutant SOS2 genes/proteins: T/DSOS2 (polynucleotide sequence=SEQ ID NO: 4; protein sequence=SEQ ID NO: 5), T/DSOS2/308 (polynucleotide sequence=SEQ ID NO: 6; protein sequence=SEQ ID NO: 7), T/DSOS2/329 (polynucleotide sequence=SEQ ID NO: 8; protein sequence=SEQ ID NO: 9), and T/DSOS2DF (polynucleotide sequence=SEQ ID NO: 10; protein sequence=SEQ ID NO: 11). When endogenous polynucleotides and/or proteins are employed, these sequences should encode for a protein possessing or should possess, respectively, serine/threonine kinase activity. In certain embodiments, the polynucleotide of the present disclosure encodes the calcium binding protein SOS3 having a native polynucleotide sequence of SEQ ID NO: 12 and a protein sequence of SEQ ID NO: 13. Of course, the present disclosure also includes homologues, derivatives or active fragments of all of the SEQ IDs disclosed above.

CAX

In some embodiments, the antiporter is a CAX antiporter. Ca²⁺/cation antiporter (CaCA) proteins are integral membrane proteins that transport Ca²⁺ or other cations using the H⁺ or Na⁺ gradient generated by primary transporters. The CAX (for CAtion eXchanger) family is one of the five families that make up the CaCA superfamily. CAX genes have been found in bacteria, Dictyostelium, fungi, plants, and lower vertebrates. It has been demonstrated that there are three major types of CAXs: type I (CAXs similar to Arabidopsis thaliana CAX1, found in plants, fungi, and bacteria), type II (CAXs with a long N-terminus hydrophilic region, found in fungi, Dictyostelium, and lower vertebrates), and type III (CAXs similar to Escherichia coli ChaA, found in bacteria) (for example, as described in Shigaki, T., et al.; Journal of molecular evolution (2006) vol. 63, no 6, pp. 815-825). Some CAXs have secondary structures that are different from the canonical six transmembrane (TM) domains-acidic motif-five TM domain structure.

Cation/Ca²⁺ exchangers are an essential component of Ca²⁺ signaling pathways and function to transport cytosolic Ca²⁺ across membranes against its electrochemical gradient by utilizing the downhill gradients of other cation species such as H⁺, Na⁺ or K⁺. The cation/Ca²⁺ exchanger superfamily is composed of H⁺/Ca²⁺ exchangers and Na⁺/Ca²⁺ exchangers, which have been investigated extensively in both plant cells and animal cells. Information from completely sequenced genomes of bacteria, archaea, and eukaryotes has revealed the presence of genes that encode homologues of cation/Ca²⁺ exchangers in many organisms in which the role of these exchangers has not been clearly demonstrated. A comprehensive sequence alignment and the first phylogenetic analysis of the cation/Ca²⁺ exchanger superfamily of 147 sequences has been reported (Cai, X., Mol. Biol. Evol. (2004) 21(9):1692-1703). These results present a framework for structure-function relationships of cation/Ca²⁺ exchangers, suggesting unique signature motifs of conserved residues that may underlie divergent functional properties. Construction of a phylogenetic tree with inclusion of cation/Ca²⁺ exchangers with known functional properties defines five protein families and the evolutionary relationships between the members. Based on the analysis discussed above (Cai, X., Mol. Biol. Evol. (2004) 21(9):1692-1703), the cation/Ca²⁺ exchanger superfamily is classified into the YRBG, CAX, NCX, and NCKX families, and a newly recognized family, designated CCX. These findings provide guides for future studies concerning structures, functions, and evolutionary origins of the cation/Ca²⁺ exchangers.

ENA1

In some embodiments, the polynucleotide encodes an ENA1. A plasma membrane located sodium ATPase named ENA1 seems to play a role in the sodium tolerance of fungi. ENA1 is thought to be ubiquitous in all fungi, and homologs of this protein have also been described in other systems, for example, moss (e.g. Physcomitrella patens).

In addition, the role of ENA1 in the regulation of salt tolerance in yeast has been studied. ENA1 activity is reported to be one of the primary modes of Na+ efflux from yeast cells and the deletion of ENA1 led to the loss of yeast growth at 500 mM NaCl (Ruiz, A. and Arino, J. (2007), Eukaryotic Cell, p. 2175-2183). Expression of PpENA1 (a sodium ATPase) is able to complement a highly salt sensitive phenotype in yeast cells indicating the importance of ENA proteins in sodium efflux in yeast (Benito, B., Plant J. (2003) 36(3):382-389).

ENA1 is discussed, for example, in Serrano, R. and Rodriguez-Navarro, A., Curr Opin Cell Biol. (2001) 13(4):399-404. In yeast, a transcription repressor, Sko1, mediates regulation of the sodium-pump ENA1 gene by the Hog1 MAP kinase.

The regulatory subunit of S. cerevisiae casein kinase II (CKII) is encoded by two genes, CKB1 and CKB2. Strains harboring deletions of either or both genes exhibit specific sensitivity to high concentrations of Na+ or Li+. Na+ tolerance in S. cerevisiae is mediated primarily by transcriptional induction of ENA1, which encodes the plasma membrane sodium pump, and by conversion of the potassium uptake system to a higher affinity form that discriminates more efficiently against Na⁺. To determine whether reduced ENA1 expression plays a role in the salt sensitivity of ckb mutants. Tenney, K. A. and Glover, C. V. C. (1999, Molecular and Cellular Biochemistry, 191:161-167) integrated an ENA1-lacZ reporter gene into isogenic wild-type, ckb1, ckb2, and ckb1 ckb2 strains and monitored beta-galactosidase activity at different salt concentrations. In all three mutants transcription from the ENA1 promoter remained salt-inducible, but both basal and salt-induced expression was depressed approximately 3- to 4-fold. The degree of reduction in ENA1 expression was comparable to that observed in an isogenic strain carrying a null mutation in protein phosphatase 2B (calcineurin), which is also required for salt tolerance. These results suggest that reduced expression of ENA1 contributes to the salt sensitivity of ckb strains. Consistent with this conclusion, over expression of ENA1 from an exogenous promoter (GAL1) completely suppressed the salt sensitivity of ckb mutants. Induction of ENA1 expression by alkaline pH is also depressed in ckb mutants, but unlike calcineurin mutants, ckb strains are not growth inhibited by alkaline pH.

Therefore, fungal, yeast, or moss sodium ATPases are additional candidate genes to be engineered into organisms, such as Chlamydomonas, to improve salt tolerance.

H+-pyrophosphatase: AVP1

In some embodiments, the polynucleotide encodes an H+-pyrophosphatase. Vacuolar proton pyrophosphatases (V-H(+)-PPases) are electrogenic proton pumps found in many organisms of considerable industrial, environmental, and clinical importance.

The heterologous expression of Arabidopsis H-PPase was shown to enhance salt tolerance in transgenic creeping bentgrass (Agrostis stolonifera L.) (Li, Z., et al., Plant Cell Environ. 2009 Nov. 17, Abstract).

In addition, the heterologous expression of vacuolar H(+)-PPase has been shown to enhance the electrochemical gradient across the vacuolar membrane and improve tobacco cell salt tolerance (Duan, X. G., et al., Protoplasma. 2007; 232(1-2):87-95).

V-H(+)-PPases of several parasites were shown to be associated with acidic vacuoles named acidocalcisomes, which contain polyphosphate and calcium (Lemercier, G., et al., J. Biol. Chem. (2002) 277(40):37369-37376).

The vacuolar H+ pyrophosphatase of mung bean has been cloned and characterized by Nakanishi, Y. and Maeshima, M., Plant Physiology (1998) 116:589-597.

Some transgenic plants over expressing a vacuolar H⁺-pyrophosphatase are much more resistant to high concentrations of NaCl and to water deprivation than the isogenic wild-type strains. These transgenic plants accumulate more Na⁺ and K⁺ in their leaf tissue than their wild type counterparts.

In some embodiments, the H+-pyrophosphoatase is AVP1 or a functional homolog thereof. Overexpression of the vacuolar H⁺-pyrophosphatase (H⁺-PPase) AVP1 in the model plant Arabidopsis thaliana resulted in enhanced performance under soil water deficits (Park S. et al., PNAS (2005) vol. 102 no. 52, pages 18830-5). Direct measurements on isolated vacuolar membrane vesicles derived from AVP1 transgenic plants and from wild type demonstrated that the vesicles from the transgenic plants had enhanced cation uptake (Gaxiola, R. A., et al. PNAS (2001) vol. 98 no. 20 11444-11449). The phenotypes of the AVP1 transgenic plants suggest that increasing the vacuolar proton gradient results in increased solute accumulation and water retention. AVP1 is also able to significantly overcome the ENA1 protein deficiency in yeast in the presence of the NHX1 protein (Gaxiola, R. A., et al., Proc. Natl. Acad. Sci. USA (1999) 96(4):1480-1485). Thus, AVP1 gene is another exemplary candidate gene that can be over expressed in an organism, such as Chlamydomonas, along with a NHX1 homolog to confer salt resistance.

In some embodiments, the transgenic alga expresses a transporter that confers salt tolerance to the transgenic alga. In some embodiments, the transporter transports Li+, Na+, or K+. The transporter can be an ATPase including, but not limited to, a Na+ ATPase, a Li+ ATPase, or a P-type ATPase. The P-type ATPase can be ENA1 or a functional homolog of ENA1. In some embodiments, the transporter is an antiporter including, but not limited to, a Na+ antiporter, a CAX antiporter, a NHX antiporter, or a functional homolog of any of the above. The transporter can also be an SOS1 protein, a Nha protein, or a Nap protein, or a functional homolog of any of the above. In some embodiments, the exogenous or endogenous polynucleotide encodes a H+-pyrophosphatase, for example, AVP1 or a functional homolog of AVP1. The exogenous or endogenous polynucleotide may encode a protein that regulates the expression of a transporter. Examples of such regulators include, but are not limited to, an SOS2 protein, an SOS3 protein, or a functional homolog of either of the above.

More than one gene involved in salt tolerance can be introduced into the organism to confer salt tolerance to the organism. In some embodiments, a transgenic alga comprises two or more exogenous or endogenous polynucleotides, wherein each of the exogenous or endogenous polynucleotides encodes an ATPase, an antiporter, or an H+-pyrophosphatase. The present disclosure also encompasses a transgenic alga comprising a first exogenous or endogenous polynucleotide encoding an ATPase, and a second exogenous or endogenous polynucleotide encoding an antiporter. For example, a transgenic alga can comprise a first exogenous or endogenous polynucleotide encoding a plasma membrane ATPase and a second exogenous or endogenous polynucleotide encoding a vacuolar antiporter. In another example, a transgenic alga can comprise a first exogenous or endogenous polynucleotide encoding a plasma membrane ATPase and a second exogenous or endogenous polynucleotide encoding a plasma membrane antiporter. A transgenic alga may also comprise a first exogenous or endogenous polynucleotide encoding a H+-pyrophosphatase and second exogenous or endogenous polynucleotide encoding an antiporter. In some instances, a transgenic alga comprises a first exogenous or endogenous polynucleotide encoding a vacuolar H+-pyrophosphatase and a second exogenous or endogenous polynucleotide encoding a vacuolar antiporter. A transgenic alga may further comprise a third exogenous or endogenous polynucleotide encoding a vacuolar chloride channel protein.

In some embodiments, a transgenic alga comprises an exogenous or endogenous polynucleotide encoding a bbc protein or a functional homolog thereof, a SCSR protein or a functional homolog thereof, a chaperonin, or an antioxidant enzyme. Antioxidant enzymes provide an important defense against free radicals. Examples of antioxidant enzymes that can be used in this disclosure include, but are not limited to, any one or more of glutathione peroxidase, glutathione reductase, ascorbate peroxidase, catalase, alternative oxidase, and superoxide dismutase.

Examples of genes and proteins that confer salt tolerance and that can be used in the embodiments disclosed herein include, but are not limited to: glutathione peroxidase (GPX) from various organisms, for example, CW80GPX from Chlamydomonas sp. W80 (Takeda, T. M., et al., Physiol Plant (2003) 117(4):467-475, Molecular characterization of glutathione peroxidase-like protein in halotolerant Chlamydomonas sp. W80) (SEQ ID NO: 26 (DNA) and SEQ ID NO: 27 (protein)); CrGPX5 from Chlamydomonas reinhardtii (SEQ ID NO: 14 (DNA) and SEQ ID NO: 32 (protein)); ScGPX1 from Schizosaccharomyces pombe (SEQ ID NO: 36); a Na+/H+ antiporter Nhx1, for example, AgNHX1 from Atriplex gmelini (SEQ ID NO: 40) (Hamada, A., et al., Plant Mol Biol. (2001) 46(1):35-42, Isolation and characterization of a Na+/H+ antiporter gene from the halophyte Atriplex gmelini); AtNHX1 from Arabidopsis thaliana (SEQ ID NO: 44); AtSOS1 from Arabidopsis thaliana (SEQ ID NO: 48); CW80BBC1; a 60S ribosomal protein L13 from Chlamydomonas sp. W80 (SEQ ID NO: 51 (DNA) and SEQ ID NO: 52 (protein)); and a CW80 scsr protein from Chlamydomonas sp. W80 (SEQ ID NO: 56 (protein) and SEQ ID NO: 55 (DNA)).

Examples of genes and proteins that can be used in the embodiments disclosed herein include, but are not limited to:

SEQ ID NO: 1 is the native nucleic acid sequence for NHX1 from Oryza sativa.

SEQ ID NO: 2 is the native protein sequence of NHX1 from Oryza sativa.

SEQ ID NO: 3 is the native nucleic acid sequence for NHX1 from Arabidopsis thaliana.

SEQ ID NO: 4 is the nucleic acid sequence for T/DSOS2 a truncated version of the native SOS2 protein sequence from Arabidopsis thaliana.

SEQ ID NO: 5 is the protein sequence of T/DSOS2 a truncated version of the native SOS2 protein sequence from Arabidopsis thaliana.

SEQ ID NO: 6 is the nucleic acid sequence of T/DSOS2/308 a truncated version of the native SOS2 nucleic acid sequence from Arabidopsis thaliana.

SEQ ID NO: 7 is the protein sequence of T/DSOS2/308 a truncated version of the native SOS2 protein sequence from Arabidopsis thaliana.

SEQ ID NO: 8 is the nucleic acid sequence of T/DSOS2/329 a truncated version of the native SOS2 nucleic acid sequence from Arabidopsis thaliana.

SEQ ID NO: 9 is the protein sequence of T/DSOS2/329 a truncated version of the native SOS2 protein sequence from Arabidopsis thaliana.

SEQ ID NO: 10 is the nucleic acid sequence of TiDSOS2DF a truncated version of the native SOS2 nucleic acid sequence from Arabidopsis thaliana.

SEQ ID NO: 11 is the protein sequence of T/DSOS2DF a truncated version of the native SOS2 protein sequence from Arabidopsis thaliana.

SEQ ID NO: 12 is the native nucleic acid sequence for SOS3 from Arabidopsis thaliana.

SEQ ID NO: 13 is the native protein sequence of SOS3 from Arabidopsis thaliana.

SEQ ID NO: 14 is the native nucleic acid sequence for glutathione peroxidase from Chlamydomonas reinhardtii.

SEQ ID NO: 15 is the native nucleic acid sequence for glutathione peroxidase from Chlamydomonas reinhardtii, modified to remove SalI and NheI restriction sites.

SEQ ID NO: 16 is the native protein sequence of Glutathione-Dependent Phospholipid Peroxidase Hyr1 from Saccharomyces Cerevisiae.

SEQ ID NO: 17 is the native nucleic acid sequence for CW80Cd404 protein from Chlamydomonas sp. W80.

SEQ ID NO: 18 is a synthetic (codon optimized) nucleic acid sequence for GPX5 from Chlamydomonas reinhardtii.

SEQ ID NO: 19 is a synthetic (codon optimized) nucleic acid sequence for GPX1 from S. Pombe.

SEQ ID NO: 20 is a synthetic (codon optimized) nucleic acid sequence for NHX1 from A. gmelini.

SEQ ID NO: 21 is a synthetic (codon optimized) nucleic acid sequence for NHX1 from Arabidopsis thaliana.

SEQ ID NO: 22 is a synthetic (codon optimized) nucleic acid sequence for SOS1 from Arabidopsis thaliana.

SEQ ID NO: 23 is a synthetic (codon optimized) nucleic acid sequence for BBC1 from Chlamydomonas sp. W80.

SEQ ID NO: 24 is a synthetic (codon optimized) nucleic acid sequence for GPX from Chlamydomonas sp. W80 (SR1) with a FLAG-TEV-MAT tag.

SEQ ID NO: 25 is the protein sequence for GPX from Chlamydomonas sp. W80 (SR1) with a FLAG-TEV-MAT tag.

SEQ ID NO: 26 is a synthetic (codon optimized) nucleic acid sequence for GPX from Chlamydomonas sp. W80 (SR1).

SEQ ID NO: 27 is the protein sequence for GPX from Chlamydomonas sp. W80 (SR1).

SEQ ID NO: 28 is the protein sequence for FLAG-TEV-MAT tag.

SEQ ID NO: 29 is the synthetic (codon optimized) nucleic acid sequence for GPX5 from Chlamydomonas reinhardtii (SR2) with a FLAG-TEV-MAT tag.

SEQ ID NO: 30 is the protein sequence for GPX5 from Chlamydomonas reinhardtii (SR2) with a FLAG-TEV-MAT tag.

SEQ ID NO: 31 is the synthetic (codon optimized) nucleic acid sequence for GPX5 from Chlamydomonas reinhardtii (SR2).

SEQ ID NO: 32 is the protein sequence for GPX5 from Chlamydomonas reinhardtii (SR2).

SEQ ID NO: 33 is the synthetic (codon optimized) nucleic acid sequence for GPX1 from S. Pombe (SR3) with a FLAG-TEV-MAT tag.

SEQ ID NO: 34 is the protein sequence for GPX1 from S. Pombe (SR3) with a FLAG-TEV-MAT tag.

SEQ ID NO: 35 is the synthetic (codon optimized) nucleic acid sequence for GPX1 from S. Pombe (SR3).

SEQ ID NO: 36 is the protein sequence for GPX1 from S. Pombe (SR3).

SEQ ID NO: 37 is the synthetic (codon optimized) nucleic acid sequence for NHX1 from A. gmelini (SR4) with a FLAG-TEV-MAT tag.

SEQ ID NO: 38 is the protein sequence for NHX1 from A. gmelini (SR4) with a FLAG-TEV-MAT tag.

SEQ ID NO: 39 is the synthetic (codon optimized) nucleic acid sequence for NHX1 from A. gmelini (SR4).

SEQ ID NO: 40 is the protein sequence for NHX1 from A. gmelini (SR4).

SEQ ID NO: 41 is the synthetic (codon optimized) nucleic acid sequence for NHX1 from A. thaliana (SR5) with a FLAG-TEV-MAT tag.

SEQ ID NO: 42 is the protein sequence for NHX1 from A. thaliana (SR5) with a FLAG-TEV-MAT tag.

SEQ ID NO: 43 is the synthetic (codon optimized) nucleic acid sequence for NHX1 from A. thaliana (SR5).

SEQ ID NO: 44 is the protein sequence for NHX1 from A. thaliana (SR5).

SEQ ID NO: 45 is the synthetic (codon optimized) nucleic acid sequence for SOS1 from Arabidopsis thaliana (SR6) with a FLAG-TEV-MAT tag.

SEQ ID NO: 46 is the protein sequence for SOS1 from Arabidopsis thaliana (SR6) with a FLAG-TEV-MAT tag.

SEQ ID NO: 47 is the synthetic (codon optimized) nucleic acid sequence for SOS1 from Arabidopsis thaliana (SR6).

SEQ ID NO: 48 is the protein sequence for SOS1 from Arabidopsis thaliana (SR6).

SEQ ID NO: 49 is the synthetic (codon optimized) nucleic acid sequence for BBC1 from Chlamydomonas sp. W80 (SR7) with a FLAG-TEV-MAT tag.

SEQ ID NO: 50 is the protein sequence for BBC1 from Chlamydomonas sp. W80 (SR7) with a FLAG-TEV-MAT tag.

SEQ ID NO: 51 is the synthetic (codon optimized) nucleic acid sequence for BBC from Chlamydomonas sp. W80 (SR7).

SEQ ID NO: 52 is the protein sequence for BBC1 from Chlamydomonas sp. W80 (SR7).

SEQ ID NO: 53 is the synthetic (codon optimized) nucleic acid sequence for CW80Cd404 from Chlamydomonas sp. W80 (SR8) with a FLAG-TEV-MAT tag.

SEQ ID NO: 54 is the protein sequence for CW80Cd404 from Chlamydomonas sp. W80 (SR8) with a FLAG-TEV-MAT tag.

SEQ ID NO: 55 is the synthetic (codon optimized) nucleic acid sequence for CW80Cd404 from Chlamydomonas sp. W80 (SR8).

SEQ ID NO: 56 is the protein sequence for CW80Cd404 from Chlamydomonas sp. W80 (SR8).

SEQ ID NO: 57 is the native nucleic acid sequence of a predicted protein: voltage-dependent potassium channel, protein ID: 189793 from Chlamydomonas reinhardtii.

SEQ ID NO: 58 is the native protein sequence of a predicted protein: voltage-dependent potassium channel, protein ID: 189793 from Chlamydomonas reinhardtii.

SEQ ID NO: 59 is the synthetic (codon optimized) nucleotide sequence of a predicted protein: voltage-dependent potassium channel, protein ID: 189793 from Chlamydomonas reinhardtii.

SEQ ID NO: 60 is the synthetic (codon optimized) nucleotide sequence of a predicted protein: voltage-dependent potassium channel, protein ID: 189793 from Chlamydomonas reinhardtii with a restriction site engineered into the 5′ end and a FL AG-TEV-MAT tag at the 3′ end of the sequence.

SEQ ID NO: 61 is the protein sequence of a predicted protein: voltage-dependent potassium channel, protein ID: 189793 from Chlamydomonas reinhardtii with a restriction site engineered into the 5′ end and a FLAG-TEV-MAT tag at the 3′ end of the sequence.

SEQ ID NO: 62 is the FLAG-TEV-MAT tag used in SEQ ID NO: 61.

A homolog useful in the present disclosure may have at least 50%, at least 60%, at least 70%, at least 75%, at least 80%, at least 85%, at least 90%, at least 95%, at least 98%, or at least 99% sequence identity to, for example, the amino acid sequence of SEQ ID. NO: 2.

Percent Sequence Identity

One example of an algorithm that is suitable for determining percent sequence identity or sequence similarity between nucleic acid or polypeptide sequences is the BLAST algorithm, which is described, e.g., in Altschul et al., J. Mol. Biol. 215:403-410 (1990). Software for performing BLAST analysis is publicly available through the National Center for Biotechnology Information. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (as described, for example, in Henikoff & Henikoff (1989) Proc. Natl. Acad. Sci. USA, 89:10915). In addition to calculating percent sequence identity, the BLAST algorithm also can perform a statistical analysis of the similarity between two sequences (for example, as described in Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, less than about 0.01, or less than about 0.001.

Reduced or Catabolizable Carbon Sources

A “catabolizable carbon source” is a complex molecule, including but not limited to a mono- or oligo-saccharide, an amino acid, or other biochemical molecule, that can undergo catabolism in a biological cell.

Examples of catabolizable carbon sources which can be used in the described embodiments include, but are not limited to, glucose, maltose, sucrose, hydrolyzed starch, molasses, potato extract, malt, peat, vegetable oil, corn steep liquor, fructose, syrup, sugar, liquid sugar, invert sugar, alcohol, organic acid, organic acid salts, alkanes, and other general carbon sources known to one of skill in the art. These sources may be used individually or in combination.

A “reduced carbon source” is any molecule in which the average carbon oxidation state is more reduced than in a carbohydrate. Reduced carbon molecules are a subset of catabolizable carbon sources.

Examples of reduced carbon sources which can be used in the described embodiments include, but are not limited to, lipids, acetate, or amino acids.

Reduction is the gain of electrons/hydrogen or a loss of oxygen/decrease in oxidation state by a molecule, atom or ion. Photosynthesis involves the reduction of carbon dioxide into sugars and the oxidation of water into molecular oxygen. The reverse reaction, respiration, oxidizes sugars to produce carbon dioxide and water. As intermediate steps, the reduced carbon compounds are used to reduce nicotinamide adenine dinucleotide (NAD⁺), which then contributes to the creation of a proton gradient, which drives the synthesis of adenosine triphosphate (ATP) and is maintained by the reduction of oxygen.

In one aspect, the present disclosure provides an expression vector comprising a polynucleotide encoding a transporter, a protein that regulates the expression of a transporter, or a polynucleotide encoding a protein that confers salt tolerance to an organism, wherein the polynucleotide is codon biased or optimized for the nuclear genome of an algal host, wherein the transporter does not transport a reduced carbon source and/or does not transport a catabolizable carbon source.

The disclosure also provides an expression vector comprising a polynucleotide encoding a transporter or a protein that regulates the expression of a transporter, operably linked to an exogenous promoter that functions in an algal cell, wherein the transporter does not transport a reduced carbon source and/or does not transport a catabolizable carbon source.

In another aspect, the present disclosure provides a transgenic alga comprising an exogenous polynucleotide encoding a transporter or a protein that regulates expression of a transporter, wherein the transporter does not transport a catabolizable carbon source and/or does not transport a catabolizable carbon source.

The present disclosure also provides a transgenic alga comprising two or more exogenous polynucleotides, wherein at least one of the exogenous polynucleotides encodes a transporter or a protein that regulates expression of a transporter, wherein the transporter does not transport a catabolizable carbon source and/or does not transport a catabolizable carbon source.

In some embodiments, the present disclosure provides an expression vector comprising a polynucleotide encoding a transporter or a protein that regulates the expression of a transporter, wherein the polynucleotide is codon biased or optimized for the chloroplast genome of an algal host, wherein the transporter does not transport a reduced carbon source and/or does not transport a catabolizable carbon source.

Also disclosed in the present disclosure is an expression vector comprising a polynucleotide encoding a non-algal transporter or a non-algal protein that regulates the expression of a transporter, operably linked to an algal regulatory sequence, wherein the transporter does not transport a reduced carbon source and/or does not transport a catabolizable carbon source.

Organisms/Host Cells

Organisms that can be transformed using the compositions and methods disclosed herein include, but are not limited to, photosynthetic microorganisms. A photosynthetic microorganism is a microorganism that is able to use photosynthesis to gain energy from light. These organisms may be prokaryotic or eukaryotic, unicellular or multicellular. Examples of photosynthetic microorganism are described below and include, but are not limited to, algae and cyanobacteria.

Examples of non-vascular photosynthetic microorganisms include bryophtyes, such as marchantiophytes or anthocerotophytes. The photosynthetic organism may be algae (for example, macroalgae or microalgae). The algae can be unicellular or multicellular algae. In some instances the alga is a cyanophyta, a rhodophyta, a chlorophyta, a phaeophyta, a bacillariophyta, a chrysophyta, a heterokontophyta, a tribophyta, a glaucophyta, a chlorarachniophyta, a euglenophyta, a haptophyta, a cryptophyta, a phytoplankton, or a dinophyta species.

The host cell can be prokaryotic. Examples of some prokaryotic organisms of the present disclosure include, but are not limited to, cyanobacteria (e.g., Nostoc, Anabaena, Spirulina, Synechococcus, Synechocystis, Athrospira, Gleocapsa, Oscillatoria, and Pseudoanabaena). In some embodiments, the host organism is a eukaryotic algae (e.g. green algae, red algae, and brown algae). In some embodiments the algae is a green algae, for example algae from the genus Tetraselmis, the genus Micractinium, the genus Desmodesmus, the genus Scenedesmus, the genus Botryococcus, the genus Chlamydomonas, the genus Haematococcus, the genus Chlorella, and the genus Dunaliella. The algae can be unicellular or multicellular algae. In particular embodiments, the organism is a diatom, for example, a diatom from the genus Phaeodactylum, the genus Cyclotella, the genus Nitzschia, and the genus Navicula. In particular embodiments, the host cell is a microalga (e.g., Chlamydomonas reinhardtii, Dunaliella salina, Ilaematococcus pluvialis, Scenedesmus spp. (Scenedesmus dimorphus, Scenedesmus obliquus), Chlorella spp., Dunaliella viridis, or Dunaliella tertiolecta). In addition, there are many species of macroalgae, for example, Cladophora glomerata and Fucus vesiculosus. In some instances, the organism is C. reinhardtii. Chlamydomonas reinhardtii is a green unicellular freshwater alga. In another embodiment, the organism is C. reinhardtii 137c.

Algae are unicellular organisms, producing oxygen by photosynthesis. Algae are useful for biotechnology applications for many reasons, including their high growth rate and tolerance to varying environmental conditions. The use of algae in a variety of industrial processes for commercially important products is known and/or has been suggested. For example, algae are useful in the production of nutritional supplements, pharmaceuticals, and natural dyes. Algae are also used as a food source for fish and crustaceans, to control agricultural pests, in the production of oxygen, in the removal of nitrogen, phosphorus, and toxic substances from sewage, and in controlling pollution, for example, algae can be used to biodegrade plastics or can be involved in the uptake of carbon dioxide. Algae, like other organisms, contain lipids and fatty acids as membrane components, storage products, metabolites and are sources of energy. Algal strains with high oil or lipid content are of great interest in the search for a sustainable feedstock for the production of biofuels.

The host organism can be a member of the genus Nannochloropsis. Nannochloropsis is a genus of alga comprising approximately six species (N. gaditana, N. granulata, N. limnetica, N. oceanica, N. oculata, and N. salina). The species have mostly been found in marine environments but also occur in fresh and brackish water. All of the species are small, nonmotile spheres which do not express any distinct morphological features, and cannot be distinguished by either light or electron microscopy. The characterization of Nannochloropsis is mostly done by rbcL gene and 18S rDNA sequence analysis. Nannochloropsis are different from other related microalgae in that they lack chlorophyll b and c. Nannochloropsis are able to build up a high concentration of a range of pigments such as astaxanthin, zeaxanthin and canthaxanthin. Nannochloropsis have a diameter of about 2 micrometers. Nannochloropsis are considered a promising alga for industrial applications because of their ability to accumulate high levels of polyunsaturated fatty acids.

Some of the host organisms which may be used are halophilic (e.g., Dunaliella salina, D. viridis, or D. tertiolecta). For example, D. salina can grow in ocean water and salt lakes (salinity from 30-300 parts per thousand) and high salinity media (e.g., artificial seawater medium, seawater nutrient agar, brackish water medium, seawater medium, etc.). In some embodiments, a host cell comprising a polynucleotide described herein can be grown in a liquid environment which is 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 31., 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, etc.) may also be present in the liquid environments.

When a halophilic organism is utilized, it may be transformed with any of the vectors described herein. For example, D. salina may be transformed with a vector which is capable of insertion into the chloroplast or nuclear genome and which contain a nucleic acid which encodes a polynucleotide disclosed herein. Transformed halophilic organisms may then be grown in high saline environments (e.g., salt lakes, salt ponds, and high-saline media).

Dunaliella, a unicellular eukaryotic alga, isolated from water/sediment of the Dead Sea, is an obligate halophile, which is an extremophile organism that thrives in environments with very high concentrations of salt. Dunaliella is an obligate phototroph, growing photoheterotrophically in media containing yeast extract and acetate (for example, as described in Mack, E. E., et al. Archives of Microbiology, Volume 160, Number 5. November, 1993). In one embodiment, the present disclosure provides a transgenic alga comprising an exogenous polynucleotide encoding a transporter or a protein that regulates expression of a transporter, wherein the polynucleotide sequence does not alter the phototrophic state of the alga. One example of such an alga is Dunaliella.

A host algae transformed to produce a polypeptide described herein can be grown on land, e.g., ponds, aqueducts, landfills, or in closed or partially closed bioreactor systems. Algae can also be grown directly in water, e.g., in oceans, seas, on lakes, rivers, reservoirs, etc. In embodiments where algae are mass-cultured, the algae can be grown in high density photobioreactors. Methods of mass-culturing algae are known in the art. For example, algae can be grown in high density photobioreactors (see, e.g., Lee et al, Biotech. Bioengineering 44:1161-1167, 1994) and other bioreactors (such as those for sewage and waste water treatments) (e.g., Sawayama et al, Appl. Micro. Biotech., 41:729-731, 1994). Additionally, algae may be mass-cultured to remove heavy metals (e.g., Wilkinson, Biotech. Letters, 11:861-864, 1989), hydrogen (e.g., U.S. Patent Application Publication No. 20030162273), and pharmaceutical compounds.

In a particular embodiment, the host cell is a plant. The term “plant” is used broadly herein to refer to a eukaryotic organism containing plastids, particularly chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like.

In other embodiments the host organism is a vascular plant. Non-limiting examples of such plants include various monocots and dicots, including high oil seed plants such as high oil seed Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nuciera), palm (Elaeis guineensis), oilnut trees such as olive (Olea europaea), sesame, and peanut (Arachis hypogaea), as well as Arabidopsis, tobacco, wheat, barley, oats, amaranth, potato, rice, tomato, and legumes (e.g., peas, beans, lentils, alfalfa, etc.).

The use of an organism, such as microalgae to express a polypeptide or protein complex provides the advantage that large populations of the microalgae can be grown, including at commercial scale (for example, Cyanotech Corp. produces spirulina microalgae products for the consumer; Kailua-Kona Hi.), thus allowing for the production and, and if needed, the isolation of large amounts of a desired product. In addition, the ability to express, for example, functional mammalian polypeptides, including protein complexes, in the chloroplasts of a plant allows for the production of crops of such plants and, therefore, the ability to conveniently produce large amounts of the polypeptides. Accordingly, methods described herein can be practiced using any plant having chloroplasts, including, for example, macroalgae, for example, marine algae and seaweeds, as well as plants that grow in soil.

In one embodiment, the present disclosure provides a transgenic alga comprising an exogenous polynucleotide encoding a transporter or a protein that regulates expression of a transporter, wherein the algal cell is an obligatory phototroph. Obligatory phototrophs are organisms that must carry out photosynthesis to acquire energy. Energy from sunlight, carbon dioxide, and water are converted into organic materials to be used in cellular functions such as biosynthesis and respiration. C. reinhardtii is not an obligatory phototroph. It can grow in the dark in the presence of an organic carbon source such as acetate (for example, as described in Lemaire, S. D., et al. Plant Physiology and Biochemistry Volume 41, Issues 6-7, June 2003, 513-521).

A method as provided herein, for example, particle bombardment, can generate algae containing chloroplasts that are genetically modified to contain a stably integrated polynucleotide (for example, as described in Hager and Bock, Appl. Microbiol. Biotechnol. 54:302-310, 2000). Accordingly, as described herein a method can further provide a transgenic (transplastomic) alga, for example C. reinhardtii, which comprises one or more chloroplasts containing a polynucleotide encoding one or more exogenous polypeptides, including polypeptides that can specifically associate to form a functional protein complex. A photosynthetic organism can comprise at least one host cell that is modified to generate a product.

Expression Vectors and Transformation

In one aspect, the present disclosure provides an expression vector comprising a polynucleotide encoding a transporter or a protein that regulates the expression of a transporter, wherein the polynucleotide is codon biased or optimized for the nuclear genome of an algal host, wherein the transporter does not transport a reduced carbon source. The transporter may be an ion transporter.

“Operably linked” means that two or more molecules are positioned with respect to each other such that they act as a single unit and effect a function attributable to one or both molecules or a combination thereof. For example, a polynucleotide encoding a polypeptide can be operatively linked to a transcriptional or translational regulatory element, in which case the element confers its regulatory effect on the polynucleotide similarly to the way in which the regulatory element would effect a polynucleotide sequence with which it normally is associated with in a cell. A regulatory element refers to a nucleotide that regulates the transcription and/or translation of a nucleic acid or the localization of a polypeptide to which it is operatively linked. A regulatory element may be native or foreign to the nucleotide sequence encoding the polypeptide. In some embodiments, the present disclosure provides an expression vector comprising a polynucleotide encoding a non-algal transporter or a non-algal protein that regulates the expression of a transporter, operably linked to an algal regulatory sequence, wherein the transporter does not transport a reduced carbon source.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR).

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available (e.g., Ambion, Inc.; Austin Tex.), as are polynucleotides containing such nucleotide analogs (Lin et al., Nucl. Acids Res. 22:5220-5234, 1994; Jellinek et al., Biochemistry 34:11363-11372, 1995; and Pagratis et al., Nature Biotechnol. 15:68-73, 1997). The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986, 1994; and Ecker and Crooke, BioTechnology 13:351360, 1995).

A recombinant nucleic acid molecule can contain two or more nucleotide sequences that are linked in a manner such that the product is not found in a cell in nature. In particular, the two or more nucleotide sequences can be operatively linked and, for example, can encode a fusion polypeptide, or can comprise an encoding nucleotide sequence and a regulatory element, for example, a PSII promoter operatively linked to a PSII 5′ UTR. A recombinant nucleic acid molecule also can be based on, but manipulated so as to be different, from a naturally occurring polynucleotide, for example, a polynucleotide having one or more nucleotide changes such that a first codon, which is normally found in the polynucleotide, is biased for chloroplast or nuclear codon usage, or such that a sequence of interest is introduced into the polynucleotide, for example, a restriction endonuclease recognition site or a splice site, a promoter, a DNA origin of replication. In one embodiment, the present disclosure provides a transgenic alga comprising two or more exogenous polynucleotides, wherein at least one of the exogenous polynucleotides encodes a transporter or a protein that regulates expression of a transporter, wherein the transporter does not transport a catabolizable carbon source. In another embodiment, the present disclosure provides a transgenic alga comprising two or more exogenous polynucleotides, wherein at least one of the exogenous polynucleotides encodes an ion transporter or a protein that regulates expression of an ion transporter.

The organisms/host cells herein can be transformed to modify and/or increase the production of a product(s) by use of an expression vector comprising a polynucleotide of interest.

The product(s) can be naturally or not naturally produced by the organism. The expression vector can encode one or more endogenous or exogenous nucleotide sequences. Examples of exogenous nucleotide sequences that can be transformed into an algal host cell include genes from bacteria, fungi, plants, photosynthetic bacteria or other algae. Examples of nucleotide sequences that can be transformed into an algal host cell include, but are not limited to, isoprenoid synthetic genes, endogenous promoters, and 5′ UTRs from rbcS2, psbA, atpA, rbcL or any other appropriate nuclear or chloroplast genes. In some instances, an exogenous sequence is flanked by two endogenous sequences that allow insertion of the exogenous sequence into a genome of the organism by homologous recombination. In some instances, an endogenous sequence is flanked by two exogenous sequences. The first and second flanking sequences can be, for example, at least 100, at least 200, at least 300, at least 400, at least 500, at least 1000, or at least 1500 nucleotides in length.

One or more codons of an encoding polynucleotide can be biased or optimized to reflect chloroplast codon usage or nuclear codon usage. Most amino acids are encoded by two or more different (degenerate) codons, and it is well recognized that various organisms utilize certain codons in preference to others.

Such preferential codon usage, which also is utilized in chloroplasts, is referred to herein as “chloroplast codon usage.” Table 1 (below) shows the chloroplast codon usage for C. reinhardtii (see U.S. Patent Application Publication No.: 2004/0014174, published Jan. 22, 2004).

TABLE 1
Chloroplast Codon Usage in Chlamydomonas reinhardtii

| | | | |
|---|---|---|---|
| UUU 34.1*(348**) | UCU 19.4(198) | UAU 23.7(242) | UGU 8.5(87) |
| UUC 14.2(145) | UCC 4.9(50) | UAC 10.4(106) | UGC 2.6(27) |
| UUA 72.8(742) | UCA 20.4(208) | UAA 2.7(28) | UGA 0.1(1) |
| UUG 5.6(57) | UCG 5.2(53) | UAG 0.7(7) | UGG 13.7(140) |
| CUU 14.8(151) | CCU 14.9(152) | CAU 11.1(113) | CGU 25.5(260) |
| CUC 1.0(10) | CCC 5.4(55) | CAC 8.4(86) | CGC 5.1(52) |
| CUA 6.8(69) | CCA 19.3(197) | CAA 34.8(355) | CGA 3.8(39) |
| CUG 7.2(73) | CCG 3.0(31) | CAG 5.4(55) | CGG 0.5(5) |
| AUU 44.6(455) | ACU 23.3(237) | AAU 44.0(449) | AGU 16.9(172) |
| AUC 9.7(99) | ACC 7.8(80) | AAC 19.7(201) | AGC 6.7(68) |
| AUA 8.2(84) | ACA 29.3(299) | AAA 61.5(627) | AGA 5.0(51) |
| AUG 23.3(238) | ACG 4.2(43) | AAG 11.0(112) | AGG 1.5(15) |
| GUU 27.5(280) | GCU 30.6(312) | GAU 23.8(243) | GGU 40.0(408) |
| GUC 4.6(47) | GCC 11.1(113) | GAC 11.6(118) | GGC 8.7(89) |
| GUA 26.4(269) | GCA 19.9(203) | GAA 40.3(411) | GGA 9.6(98) |
| GUG 7.1(72) | GCG 4.3(44) | GAG 6.9(70) | GGG 4.3(44) |

*Frequency of codon usage per 1,000 codons.
**Number of times observed in 36 chloroplast coding sequences (10,193 codons).

The term “biased” or “optimized”, when used in reference to a codon, means that the sequence of a codon in a polynucleotide has been changed such that the codon is one that is used preferentially in, for example, the chloroplasts of the organism (see Table 1), or the nuclear genome of the organism (see Table 2). “Biased” or codon “optimized” can be used interchangeably throughout the specification.

A polynucleotide that is biased for chloroplast or nuclear codon usage can be synthesized de novo, or can be genetically modified using routine recombinant DNA techniques, for example, by a site-directed mutagenesis method, to change one or more codons.

Table 1 exemplifies codons that are preferentially used in algal chloroplast genes. The term “chloroplast codon usage” is used herein to refer to such codons, and is used in a comparative sense with respect to degenerate codons that encode the same amino acid but are less likely to be found as a codon in a chloroplast gene. The term “biased”, when used in reference to chloroplast codon usage, refers to the manipulation of a polynucleotide such that one or more nucleotides of one or more codons is changed, resulting in a codon that is preferentially used in chloroplasts. Chloroplast codon bias is exemplified herein by the alga chloroplast codon bias as set forth in Table 1. The chloroplast codon bias can, but need not, be selected based on a particular plant in which a synthetic polynucleotide is to be expressed. The manipulation can be a change to a codon, for example, by a method such as site directed mutagenesis, by a method such as PCR using a primer that is mismatched for the nucleotide(s) to be changed such that the amplification product is biased to reflect chloroplast codon usage, or can be the de novo synthesis of polynucleotide sequence such that the change (bias) is introduced as a consequence of the synthesis procedure.

In addition to utilizing chloroplast codon bias as a means to provide efficient translation of a polypeptide, it will be recognized that an alternative means for obtaining efficient translation of a polypeptide in a chloroplast is to re-engineer the chloroplast genome (e.g., a C. reinhardtii chloroplast genome) for the expression of tRNAs not otherwise expressed in the chloroplast genome. Such an engineered algae expressing one or more exogenous tRNA molecules provides the advantage that it would obviate a requirement to modify every polynucleotide of interest that is to be introduced into and expressed from a chloroplast genome; instead, algae such as C. reinhardtii that comprise a genetically modified chloroplast genome can be provided and utilized for efficient translation of a polypeptide according to any method of the disclosure. Correlations between tRNA abundance and codon usage in highly expressed genes is well known (for example, as described in Franklin et al., Plant J. 30:733-744, 2002; Dong et al., J. Mol. Biol. 260:649-663, 1996; Duret, Trends Genet. 16:287-289, 2000; Goldman et. al., J. Mol. Biol. 245:467-473, 1995; and Komar et. al., Biol. Chem. 379:1295-1300, 1998). In E. coli, for example, re-engineering of strains to express underutilized tRNAs resulted in enhanced expression of genes which utilize these codons (see Novy et al., in Novations 12:1-3, 2001). Utilizing endogenous tRNA genes, site directed mutagenesis can be used to make a synthetic tRNA gene, which can be introduced into chloroplasts to complement rare or unused tRNA genes in a chloroplast genome, such as a C. reinhardtii chloroplast genome.

Generally, the chloroplast codon bias selected for purposes of the present disclosure, including, for example, in preparing a synthetic polynucleotide as disclosed herein reflects chloroplast codon usage of a plant chloroplast, and includes a codon bias that, with respect to the third position of a codon, is skewed towards A/T, for example, where the third position has greater than about 66% AT bias, or greater than about 70% AT bias. In one embodiment, the chloroplast codon usage is biased to reflect alga chloroplast codon usage, for example, C. reinhardtii, which has about 74.6% AT bias in the third codon position.

Table 2 exemplifies codons that are preferentially used in algal nuclear genes. The term “nuclear codon usage” is used herein to refer to such codons, and is used in a comparative sense with respect to degenerate codons that encode the same amino acid but are less likely to be found as a codon in a nuclear gene. The term “biased”, when used in reference to nuclear codon usage, refers to the manipulation of a polynucleotide such that one or more nucleotides of one or more codons is changed, resulting in a codon that is preferentially used in the nucleas. Nuclear codon bias is exemplified herein by the alga nuclear codon bias as set forth in Table 2. The nuclear codon bias can, but need not, be selected based on a particular plant in which a synthetic polynucleotide is to be expressed. The manipulation can be a change to a codon, for example, by a method such as site directed mutagenesis, by a method such as PCR using a primer that is mismatched for the nucleotide(s) to be changed such that the amplification product is biased to reflect nuclear codon usage, or can be the de novo synthesis of polynucleotide sequence such that the change (bias) is introduced as a consequence of the synthesis procedure.

In addition to utilizing nuclear codon bias as a means to provide efficient translation of a polypeptide, it will be recognized that an alternative means for obtaining efficient translation of a polypeptide in a nucleus is to re-engineer the nuclear genome (e.g., a C. reinhardtii nuclear genome) for the expression of tRNAs not otherwise expressed in the nuclear genome. Such an engineered algae expressing one or more exogenous tRNA molecules provides the advantage that it would obviate a requirement to modify every polynucleotide of interest that is to be introduced into and expressed from a nuclear genome; instead, algae such as C. reinhardtii that comprise a genetically modified nuclear genome can be provided and utilized for efficient translation of a polypeptide according to any method of the disclosure. Correlations between tRNA abundance and codon usage in highly expressed genes is well known (for example, as described in Franklin et al., Plant J. 30:733-744, 2002; Dong et al., J. Mol. Biol. 260:649-663, 1996; Duret, Trends Genet. 16:287-289, 2000; Goldman et. al., J. Mol. Biol. 245:467-473, 1995; and Komar et. al., Biol. Chem. 379:1295-1300, 1998). In E. coli, for example, re-engineering of strains to express underutilized tRNAs resulted in enhanced expression of genes which utilize these codons (see Novy et al., in Novations 12:1-3, 2001). Utilizing endogenous tRNA genes, site directed mutagenesis can be used to make a synthetic tRNA gene, which can be introduced into the nucleus to complement rare or unused tRNA genes in a nuclear genome, such as a C. reinhardtii nuclear genome.

Generally, the nuclear codon bias selected for purposes of the present disclosure, including, for example, in preparing a synthetic polynucleotide as disclosed herein, can reflect nuclear codon usage of an algal nucleus and includes a codon bias that results in the coding sequence containing greater than 60% G/C content.

TABLE 2
Nuclear Codon Usage in Chlamydomonas reinhardtii
fields: [triplet] [frequency: per thousand] ([number])

| | | | |
|---|---|---|---|
| UUU 5.0(2110) | UCU 4.7(1992) | UAU 2.6(1085) | UGU 1.4(601) |
| UUC 27.1(11411) | UCC 16.1(6782) | UAC 22.8(9579) | UGC |
| | | | 13.1(5498) |
| UUA 0.6(247) | UCA 3.2(1348) | UAA 1.0(441) | UGA 0.5(227) |
| UUG 4.0(1673) | UCG 16.1(6763) | UAG 0.4(183) | UGG |
| | | | 13.2(5559) |
| CUU 4.4(1869) | CCU 8.1(3416) | CAU 2.2(919) | CGU 4.9(2071) |
| CUC 13.0(5480) | CCC 29.5(12409) | CAC 17.2(7252) | CGC |
| | | | 34.9(14676) |
| CUA 2.6(1086) | CCA 5.1(2124) | CAA 4.2(1780) | CGA 2.0(841) |
| CUG 65.2(27420) | CCG 20.7(8684) | CAG 36.3(15283) | CGG |
| | | | 11.2(4711) |
| AUU 8.0(3360) | ACU 5.2(2171) | AAU 2.8(1157) | AGU 2.6(1089) |
| AUC 26.6(11200) | ACC 27.7(11663) | AAC 28.5(11977) | AGC |
| | | | 22.8(9590) |
| AUA 1.1(443) | ACA 4.1(1713) | AAA 2.4(1028) | AGA 0.7(287) |
| AUG 25.7(10796) | ACG 15.9(6684) | AAG 43.3(18212) | AGG 2.7(1150) |
| GUU 5.1(2158) | GCU 16.7(7030) | GAU 6.7(2805) | GGU 9.5(3984) |
| GUC 15.4(6496) | GCC 54.6(22960) | GAC 41.7(17519) | GGC |
| | | | 62.0(26064) |
| GUA 2.0(857) | GCA 10.6(4467) | GAA 2.8(1172) | GGA 5.0(2084) |
| GUG 46.5(19558) | GCG 44.4(18688) | GAG 53.5(22486) | GGG 9.7(4087) |

Coding GC 66.30% 1st letter GC 64.80% 2nd letter GC 47.90% 3rd letter GC 86.21%

The term “exogenous” is used herein in a comparative sense to indicate that a nucleotide sequence (or polypeptide) being referred to is from a source other than a reference source, or is linked to a second nucleotide sequence (or polypeptide) with which it is not normally associated, or is modified such that it is in a form that is not normally associated with a reference material.

The chloroplasts of higher plants and algae likely originated by an endosymbiotic incorporation of a photosynthetic prokaryote into a eukaryotic host. During the integration process genes were transferred from the chloroplast to the host nucleus (for example, as described in Gray, Curr. Opin. Gen. Devel. 9:678-687, 1999). As such, proper photosynthetic function in the chloroplast requires both nuclear encoded proteins and plastid encoded proteins, as well as coordination of gene expression between the two genomes. Expression of nuclear and chloroplast encoded genes in plants is acutely coordinated in response to developmental and environmental factors.

In chloroplasts or the nucleus, regulation of gene expression generally occurs after transcription, and often during translation initiation. This regulation is dependent upon the chloroplast translational apparatus, as well as nuclear-encoded regulatory factors (see, for example, Barkan and Goldschmidt-Clermont, Biochemie 82:559-572, 2000; Zerges, Biochemie 82:583-601, 2000; and Bruick, R. K. and Mayfield, S. P., Trends Plant Sci. (1999) 4(5)190-195). The chloroplast translational apparatus generally resembles that of bacteria; chloroplasts contain 70S ribosomes; have mRNAs that lack 5′ caps and generally do not contain 3′ poly-adenylated tails (Harris et al., Microbiol. Rev. 58:700-754, 1994); and translation is inhibited in chloroplasts and in bacteria by selective agents such as chloramphenicol.

One approach to construction of a genetically manipulated strain of alga involves transformation with a nucleic acid which encodes a gene of interest, typically an enzyme capable of converting a precursor into a fuel product or into a precursor of a fuel product. In some embodiments, a transformation may introduce nucleic acids into any plastid of the host alga cell (for example, chloroplast). Transformed cells are typically plated on selective media following introduction of exogenous nucleic acids. This method may also comprise several steps for screening. Initially, a screen of primary transformants is typically conducted to determine which clones have proper insertion of the exogenous nucleic acids. Clones which show the proper integration may be patched and re-screened to ensure genetic stability. Such methodology ensures that the transformants contain the genes of interest. In many instances, such screening is performed by polymerase chain reaction (PCR); however, any other appropriate technique known in the art may be utilized. Many different methods of PCR are known in the art (for example, nested PCR, and real time PCR). Particular examples are utilized in the examples described herein; however, one of skill in the art will recognize that other PCR techniques may be substituted for the particular protocols described. Following screening for clones with proper integration of exogenous nucleic acids, typically clones are screened for the presence of the encoded protein. Protein expression screening can be performed by Western blot analysis and/or enzyme activity assays, for example.

A recombinant nucleic acid molecule useful in a method or composition described herein can be contained in a vector. Furthermore, where a second (or more) recombinant nucleic acid molecule is used, the second recombinant nucleic acid molecule can also be contained in a vector, which can, but need not be, the same vector as that containing the first recombinant nucleic acid molecule. The vector can be any vector useful for introducing a polynucleotide into a chloroplast and may include a nucleotide sequence of chloroplast genomic DNA that is sufficient to undergo homologous recombination with the chloroplast genomic DNA, for example, a nucleotide sequence comprising about 400 to about 1500 or more substantially contiguous nucleotides of chloroplast genomic DNA. Chloroplast vectors and methods for selecting regions of a chloroplast genome for use as a vector are well known (see, for example, Bock, J. Mol. Biol. 312:425-438, 2001; Staub and Maliga, Plant Cell 4:39-45, 1992; and Kavanagh et al., Genetics 152:1111-1122, 1999, each of which is incorporated herein by reference).

The vector can also be any vector useful for introducing a polynucleotide into the nuclear genome of a cell and may include a nucleotide sequence of nuclear genomic DNA that is sufficient to undergo homologous recombination with the nuclear genomic DNA, for example, a nucleotide sequence comprising about 400 to about 1500 or more substantially contiguous nucleotides of nuclear genomic DNA.

A vector can contain one or more promoters. Promoters useful herein may come from any source (for example, viral, bacterial, fungal, protist, or animal). The promoters contemplated herein can be, for example, specific to photosynthetic organisms, non-vascular photosynthetic organisms, and vascular photosynthetic organisms (for example, algae and flowering plants). As used herein, the term “non-vascular photosynthetic organism,” refers to any macroscopic or microscopic organism, including, but not limited to, algae, cyanobacteria, and photosynthetic bacteria, which does not have a vascular system such as that found in higher plants. In some instances, the nucleic acids described herein are inserted into a vector that comprises a promoter of a photosynthetic organism, for example, an algal promoter. The promoter can be a promoter for expression in a chloroplast and/or other plastid and/or nucleus. In some instances, the nucleic acids that are inserted into the vector are chloroplast codon biased or nuclear codon biased. Examples of promoters contemplated for use in any of the compositions or methods described herein include those disclosed in US Application No. 2004/0014174. A promoter typically includes necessary nucleic acid sequences near the start site of transcription, (for example, a TATA element).

In some embodiments, the promoter is an RBCS promoter, an LHCP promoter, a tubulin promoter, or a PsaD promoter. The promoter may be an inducible promoter or a constitutive promoter. The promoter can also be a chimeric promoter. Examples of promoters include, but are not limited to, a NIT1 promoter, a CYC6 promoter, and a CA1 promoter.

The entire chloroplast genome of C. reinhardtii is available to the public on the world wide web, at the URL “biology.duke.edu/chlamy_genome/-chloro.html” (see “view complete genome as text file” link and “maps of the chloroplast genome” link), each of which is incorporated herein by reference (J. Maul, J. W. Lilly, and D. B. Stem, unpublished results; revised Jan. 28, 2002; to be published as GenBank Ace. No. AF396929; and Maul, J. E., et al. (2002) The Plant Cell, Vol. 14 (2659-2679)). Generally, the nucleotide sequence of the chloroplast genomic DNA that is selected for use is not a portion of a gene, including a regulatory sequence or coding sequence; it is not a gene that if disrupted, due to the homologous recombination event, would produce a deleterious effect with respect to the chloroplast. For example, a deleterious effect on the replication of the chloroplast genome or to a plant cell containing the chloroplast. In this respect, the website containing the C. reinhardtii chloroplast genome sequence also provides maps showing coding and non-coding regions of the chloroplast genome, thus facilitating selection of a sequence useful for constructing a vector (also described in Maul, J. E., et al. (2002) The Plant Cell, Vol. 14 (2659-2679)). For example, the chloroplast vector, p322, is a clone extending from the Eco (Eco RI) site at about position 143.1 kb to the Xho (Xho I) site at about position 148.5 kb (see, world wide web, at the URL “biology.duke.edu/chlamy_genome/chloro.html”, and clicking on “maps of the chloroplast genome” link, and “140-150 kb” link: also accessible directly on world wide web at URL “biology.duke.edu/chlam-y/chloro/chloro140.html”).

The entire nuclear genome of C. reinhardtii is described in Merchant, S. S., et al., Science (2007), 318(5848):245-250.

A vector utilized herein also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, and sequences that encode a selectable marker, for example. As such, the vector can contain, for example, one or more cloning sites such as a multiple cloning site, which can, but need not, be positioned such that an exogenous or endogenous polynucleotide can be inserted into the vector and operatively linked to a desired element. The vector also can contain a prokaryote origin of replication (ori), for example, an E. coli ori or a cosmid ori, thus allowing passage of the vector in a prokaryote host cell, as well as in a plant chloroplast, as desired.

A regulatory element or regulatory control sequence, broadly refers to a nucleotide sequence that regulates the transcription or translation of a polynucleotide or the localization of a polypeptide to which it is operatively linked. The phrases “regulatory element” and “regulatory control sequence” can be used interchangeably throughout the disclosure. Examples of regulatory elements include, but are not limited to, an RBS, a promoter, enhancer, transcription terminator, an initiation (start) codon, a splicing signal for intron excision and maintenance of a correct reading frame, a STOP codon, an amber or ochre codon, and an IRES. Additionally, a regulatory element can comprise a cell compartmentalization signal (for example, a sequence that targets a polypeptide to the cytosol, nucleus, chloroplast membrane, or cell membrane). Such signals are well known in the art and have been widely reported (see, for example, U.S. Pat. No. 5,776,689).

Any of the expression vectors herein can comprise a regulatory control sequence. A regulatory control sequence may include for example, promoter(s), operator(s), repressor(s), enhancer(s), transcription termination sequence(s), sequence(s) that regulate translation, or other regulatory control sequence(s) that are compatible with the host cell and control the expression of the nucleic acid molecules. In some cases, a regulatory control sequence includes transcriptional control sequence(s) that are able to control, modulate, or effect the initiation, elongation, and/or termination of transcription. For example, a regulatory control sequence can increase the transcription and translation rate and/or efficiency of a gene or gene product in an organism, wherein expression of the gene or gene product is upregulated resulting (directly or indirectly) in the increased production of a desired product. The regulatory control sequence may also result in the increase of production of a product by increasing the stability of a gene or gene product.

A regulatory control sequence can be exogenous or endogenous. The regulatory control sequence may encode one or more polypeptides which are enzymes that promote expression and production of a desired product(s). For example, an exogenous regulatory control sequence may be derived from another species of the same genus of the organism (for example, another algal species) and encode a synthase in an algae. In another example, an exogenous regulatory control sequence can be derived from an organism in which an expression vector comprising the regulatory control sequence is to be expressed.

Regulatory control sequences can be used that effect inducible or constitutive expression. For example, algal regulatory control sequences can be used, and can be of nuclear, viral, extrachromosomal, mitochondrial, or chloroplastic origin.

Suitable regulatory control sequences can include those naturally associated with the nucleotide sequence to be expressed (for example, an algal promoter operably linked to an algal nucleotide sequence in nature). Suitable regulatory control sequences can also include regulatory control sequences not naturally associated with the nucleic acid molecule to be expressed (for example, an algal promoter of one species operatively linked to a nucleotide sequence of another organism or algal species).

To determine whether a putative regulatory control sequence is suitable for use, the putative regulatory control sequence can be linked to a nucleic acid molecule that encodes a protein that produces an easily detectable signal. The vector, comprising the putative regulatory control sequence linked to the nucleic acid encoding a protein that produces a detectable signal, is then introduced into an alga or other organism by standard techniques and expression of the protein is monitored. For example, if the nucleic acid molecule encodes a dominant selectable marker, the alga or organism to be used is tested for the ability to grow in the presence of a compound for which the marker provides resistance.

In some cases, a regulatory control sequence is a promoter, such as a promoter adapted for expression of a nucleotide sequence in a non-vascular, photosynthetic organism. For example, the promoter may be an algal promoter, for example as described in U.S. Publ. Appl. Nos. 2006/0234368 and 2004/0014174, and in Hallmann, Transgenic Plant J. 1:81-98 (2007). The promoter may be a chloroplast specific promoter or a nuclear promoter. The promoter may be an EF1-α gene promoter or a D promoter. In some embodiments, a polynucleotide of interest is operably linked to an EF1-α gene promoter. In other embodiments, the polynucleotide of interest is operably linked to a D promoter.

A regulatory control sequence described herein can be placed in a variety of locations, including for example, coding and non-coding regions, 5′ untranslated regions (for example, regions upstream from the coding region), and 3′ untranslated regions (for example, regions downstream from the coding region). Thus, in some instances an endogenous or exogenous nucleotide sequence can include one or more 3′ or 5′ untranslated regions, one or more introns, or one or more exons. For example, in some embodiments, a regulatory control sequence can comprise a Cyclotella cryptica acetyl-CoA carboxylase 5′ untranslated regulatory control sequence or a Cyclotella cryptica acetyl-CoA carboxylase 3′-untranslated regulatory control sequence (for example, as described in U.S. Pat. No. 5,661,017).

A regulatory control sequence may also encode chimeric or fusion polypeptides, such as protein AB, or serum albumin A (SAA), that promote the expression of exogenous or endogenous proteins. Other regulatory control sequences include intron sequences that may promote the translation of an exogenous or endogenous sequence.

The regulatory control sequences used in any of the expression vectors described herein may be inducible. Inducible regulatory control sequences, such as promoters, can be inducible by light or an exogenous agent, for example. Other inducible elements are well known in the art and may be adapted for use as described herein. Regulatory control sequences may also be autoregulatable. Examples of autoregulatable regulatory control sequences include those that are autoregulated by, for example, endogenous ATP levels or by a product produced by the organism. The product may form a feedback loop, wherein when the product (for example fuel product, fragrance product, or insecticide product) reaches a certain level in the cell, expression of the product is inhibited. In other embodiments, the level of a metabolite present in the cell inhibits the expression of the product. For example, endogenous ATP produced by the cell as a result of increased energy production used to express the product, may form a feedback loop to inhibit expression of the product. In addition, an expression vector for effecting production of a product in an organism may comprise an inducible regulatory control sequence that is inactivated by an exogenous agent.

It has previously been noted that proper placement of an RBS with respect to a coding sequence, for example, a nucleic acid sequence encoding an ion transporter, results in robust translation in plant chloroplasts (for example, as described in U.S. Application 2004/0014174, incorporated herein by reference), and that an advantage of expressing polypeptides in chloroplasts is that the polypeptides do not proceed through cellular compartments typically traversed by polypeptides expressed from a nuclear genome and therefore, are not subject to certain post-translational modifications such as glycosylation.

Various regulatory control sequences described herein may be combined with other features described herein. For example, an expression vector comprising one or more regulatory control sequences is operatively linked to a nucleotide sequence encoding a polypeptide that, for example, upregulates the production of a desired product.

A vector or other polynucleotide of the present disclosure can include a nucleotide sequence encoding a polypeptide of interest or other selectable marker. The term “selectable marker” refers to a polynucleotide (or encoded polypeptide) that confers a detectable phenotype, for example, salt tolerance. For example, a selectable marker can be a polypeptide that, when present or expressed in a cell, provides a selective advantage (or disadvantage) to the cell containing the marker, for example, the ability to grow in the presence of high concentrations of salt that would otherwise kill the cell.

For example, a selectable marker can provide a means to obtain plant cells that express the specific marker (see, for example, Bock, J. Mol. Biol. 312:425-438, 2001).

Examples of selectable markers that confer salt tolerance in plants, for example, the alga C. reinhardtii include, but are not limited to, ATPases, antiporters, CAX proteins, NHX proteins, SOS1 proteins, Nha proteins, Nap proteins, H-pyrophosphatases, AVP1 proteins, SOS2 proteins, SOS3 proteins, bbc proteins, SCSR proteins, chaperonins, antioxidant enzymes, glutathione peroxidases, ascorbate peroxidases, catalases, alternative oxidases, and superoxide dismutases. A tag can be added to the 5′ or 3′ end of the nucleic acid sequence of interest so that the resulting protein can be, for example, more easily isolated or purified. For example, a Metal Affinity Tag (MAT) can be added to the 3′ end of the open reading frame (ORF), using standard techniques. In one embodiment, a transporter described herein is modified by the addition of an N-terminal strep tag epitope to add in detection of the expression of the transporter. In one embodiment, the proteins encoded by the nucleic acids described herein are modified at the C-terminus by the addition of a Flag-tag epitope to add in the detection of protein expression, and to facilitate protein purification. Affinity tags can be appended to proteins so that they can be purified from their crude biological source using an affinity technique. These include, for example, chitin binding protein (CBP), maltose binding protein (MBP), and glutathione-S-transferase (GST). The poly(His) tag is a widely-used protein tag; it binds to metal matrices. Some affinity tags have a dual role as a solubilization agent, such as MBP, and GST. Chromatography tags are used to alter chromatographic properties of the protein to afford different resolution across a particular separation technique. Often, these consist of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include, but are not limited to, V5-tag, c-myc-tag, and HA-tag. These tags are particularly useful for western blotting and immunoprecipitation experiments, although they also find use in antibody purification. Fluorescence tags are used to give visual readout on a protein. GFP and its variants are the most commonly used fluorescence tags. More advanced applications of GFP include using it as a folding reporter (fluorescent if folded, colorless if not). A tag can comprises an amino acid sequence of PGDYKDDDDKSGENLYFQGHNHRHKHTG (SEQ ID NO: 62) or TGDYKDDDDKSGENLYFQGHNHRHKHTG (SEQ ID NO: 28), for example.

A polynucleotide or nucleic acid molecule of the disclosure, which can be contained in a vector, including any vector of the disclosure, can be introduced into, for example, a plant chloroplast or plant nucleus using any method known in the art. As used herein, the term “introducing” means transferring a polynucleotide or nucleic acid into a cell, including a prokaryote or a plant cell, for example, a plant cell plastid. A polynucleotide can be introduced into a cell by a variety of methods, which are well known in the art and selected, in part, based on the particular host cell. For example, the polynucleotide can be introduced into a plant cell using a direct gene transfer method such as electroporation or microprojectile mediated (biolistic) transformation using a particle gun, the “glass bead method” (see, for example, Kindle, K. L., et al., Proc. Natl. Acad. Sci. USA (1991) 88(5): 1721-1725), vortexing in the presence of DNA-coated microfibers (Dunahay, Biotechniques, 15(3):452-458, 1993), by liposome-mediated transformation, or by transformation using wounded or enzyme-degraded immature embryos (see Potrykus, Ann. Rev. Plant. Physiol. Plant Mol. Biol. 42:205-225, 1991).

Plastid transformation is a routine and well known method for introducing a polynucleotide into a plant cell chloroplast (for example, see U.S. Pat. Nos. 5,451,513, 5,545,817, and 5,545,818; International Publication No.: WO 95/16783; and McBride et al., Proc. Natl. Acad. Sci., USA 91:7301-7305, 1994). Chloroplast transformation involves introducing regions of chloroplast DNA flanking a desired nucleotide sequence into a suitable target cell; using, for example, a biolistic or protoplast transformation method (e.g., calcium chloride or PEG mediated transformation). For example, fifty base pairs to three kilobases of flanking nucleotide sequences of chloroplast genomic DNA allow for the homologous recombination of the desired nucleotide sequence with the chloroplast genome, resulting in the replacement or modification of specific regions of the plastid genome. Using this method, point mutations in the chloroplast 16S rRNA and rps12 genes, which confer resistance to spectinomycin or streptomycin, can be utilized as selectable markers for transformation (for example, as described in Newman et al., Genetics 126:875-888, 1990; Svab et al., Proc. Natl. Acad. Sci., USA 87:8526-8530, 1990: and Staub and Maliga, Plant Cell 4:39-45, 1992), and can result in stable homoplasmic transformants, at a frequency of approximately one per 100 bombardments of target tissues. The presence of cloning sites between these markers provides a convenient nucleotide sequence for the insertion of a desired nucleic acid into a chloroplast vector (for example, as described in Staub and Maliga, EMBO J. 12:601-606, 1993), including a vector of the disclosure. Substantial increases in transformation frequency are obtained by replacement of the recessive rRNA or r-protein antibiotic resistance genes with a dominant selectable marker, for example, the bacterial aadA gene encoding the spectinomycin-detoxifying enzyme aminoglycoside-3′-adenyltransferase (for example, as described in Goldschmidt-Clermont. Nucleic Acids Res 19:4083-4389, 1991: and Svab and Maliga, Proc. Natl. Acad. Sci., USA 90:913-917, 1993). Approximately 15 to 20 cell division cycles following transformation are generally required to reach a homoplasmic state.

It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein.

A direct gene transfer method such as electroporation also can be used to introduce a polynucleotide of the disclosure into a plant protoplast (for example, as described in Fromm et al., Proc. Natl. Acad. Sci., USA 82:5824, 1985). Electroporation involves electrical impulses of high field strength reversibly permeabilizing membranes, thus allowing the introduction of the polynucleotide. Another method that can be used is microinjection, as described in Potrykus and Spangenberg (eds.), Gene Transfer To Plants (Springer Verlag, Berlin, N.Y. 1995). A transformed plant cell containing the introduced polypeptide can be identified by detecting a phenotype due to the introduced polypeptide, for example, expression of a reporter gene or a selectable marker linked to the expression of the introduced polypeptide.

Microprojectile mediated transformation also can be used to introduce a polynucleotide into a plant cell chloroplast (for example, as described in Klein et al., Nature 327:70-73, 1987) or a plant cell nucleus. This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as the BIOLISTIC PD-1000 particle gun (BioRad; Hercules Calif.). Methods for the transformation using biolistic methods are well known (for example, as described in Wan, Plant Physiol. 104:37-48, 1984; Vasil, BioTechnology 11: 1553-1558, 1993; and Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (for example, as described in Duan et al., Nature Biotech. 14:494-498, 1996; and Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, and the glass bead agitation method (for example, as described in Kindle, K. L., et al. (Proc. Natl. Acad. Sci. USA (1991) 88(5):1721-1725).

Protein of Interest

One approach to construction of a genetically manipulated strain of an organism, such as an alga, involves transformation of the alga with a polynucleotide sequence which encodes a protein of interest. In some embodiments, the transgenic alga of the disclosure comprises a first exogenous or endogenous polynucleotide encoding a transporter or a protein that regulates expression of a transporter, wherein expression of the first exogenous or endogenous polynucleotide confers salt tolerance, and a second exogenous or endogenous polynucleotide encoding a protein of interest. The protein of interest can include, but is not limited to, a therapeutic protein, a nutritional protein, an industrial enzyme, a fuel product, a fragrance product, or an insecticide product. The protein of interest can also be a protein that participates in or promotes the synthesis of at least one nutritional, therapeutic, commercial, or fuel product in an organism, for example, a photosynthetic unicellular organism. The protein of interest can also be a protein that facilitates the isolation of at least one nutritional, therapeutic, commercial, or fuel product from an organism, for example, a photosynthetic unicellular organism.

In other aspects, the present disclosure discloses a method of selecting a transformant comprising an exogenous or endogenous polynucleotide sequence encoding a protein of interest. In still other aspects, the present disclosure describes a method for producing one or more biomolecules, comprising growing transgenic alga transformed with a polynucleotide encoding an ion transporter or protein that regulates the expression of an ion transporter, at a concentration of salt that inhibits the growth of non-transformed alga, and harvesting one or more biomolecules from the alga.

Therapeutic Proteins or Products

In some embodiments, the exogenous or endogenous polynucleotide encodes a therapeutic protein or product. Therapeutic proteins are proteins that, for example, are extracted from human cells or engineered in the laboratory for pharmaceutical use. Many therapeutic proteins are recombinant human proteins manufactured using non-human mammalian cell lines that are engineered to express the therapeutic protein. Recombinant proteins are an important class of therapeutics, useful, for example, to replace deficiencies in critical blood borne growth factors and to strengthen the immune system to fight cancer and infectious disease. Therapeutic proteins are also used to relieve patients' suffering from many conditions, including, but not limited to, various cancers, heart attacks, strokes, cystic fibrosis, Gaucher's disease, diabetes (insulin), anaemia (erythropoietin), and haemophilia. Therapeutic proteins can also help prevent or slow down the onset of such conditions. Exemplary therapeutic proteins include erythropoietins, monoclonal antibodies, and interferons.

Nutritional Proteins or Products

In some embodiments, the exogenous or endogenous polynucleotide encodes a nutritional protein or product. Nutritional proteins are proteins of nutritional value. Examples of nutritional proteins include, but are not limited to, albumin, prealbumin, retinol-binding protein, and transferrin.

Industrial Enzymes or Products

In some embodiments, the exogenous or endogenous polynucleotide encodes an industrial enzyme or product. Many enzymes are used in the chemical industry. Examples of industrial enzymes that may be used in the embodiments described herein include, but are not limited to, alpha-amylase, beta-amylase, cellulase, beta-glucanase, beta-glucosidase, dextranase, dextrinase, alpha-galactosidase, glucoamylase, hemmicellulase, invertase, lactase, naringinase, pectinase, pullulanase, acid proteinase, alkaline protease, bromelain, pepsin, aminopeptidase, endo-peptidase, subtilisin, aminoacylase, glutaminase, lysozyme, penicillin acylase, isomerase, alcohol dehydrogenase, amino acid oxidase, catalase, chloroperoxidase, peroxidase, acetolactate decarboxylase, aspartic beta-decarboxylase, histidase, cyclodextrin glycosyltransferase, actinidin, ficin, lipoxygenase, papain, asparaginase, glucose isomerase, penicillin amidase, protease, glucose oxidase, lactase, lipase, Rennet, pectinase, pectin lyase, raffinase, and invertase.

Enzymes may also be used to help produce fuels from renewable sources of biomass. Such enzymes include, for example, cellulases, which convert cellulose fibers from feedstocks, like corn, into sugars. These sugars are subsequently fermented into ethanol by microorganisms. Other exemplary enzymes that can be used in the disclosed embodiments include, but are not limited to, hemicellulases, proteases, ligninases, and amylases.

Products

In some embodiments, the exogenous or endogenous polynucleotide encodes a fuel product or a protein or enzyme involved in making a fuel product. Examples of fuel products include petrochemical products and their precursors, and all other substances that may be useful in the petrochemical industry. Fuel products include, for example, petroleum products, terpenes, isoprenoids, fatty acids, triglycerides, carotenoids, petroleum, petrochemicals, and precursors of any of the above. The fuel products contemplated herein include hydrocarbon products and hydrocarbon derivative products. The fuel product may be used for generating substances, or materials, useful in the petrochemical industry, including petroleum products and petrochemicals. The fuel or fuel products may be used in a combustor such as a boiler, kiln, dryer or furnace. Other examples of combustors are internal combustion engines such as vehicle engines or generators, including gasoline engines, diesel engines, jet engines, and other types of engines. Fuel products may also be used to produce plastics, resins, fibers, elastomers, lubricants, and gels.

In some embodiments, the exogenous or endogenous polynucleotide encodes a synthase. Examples of synthases include, but are not limited to, botryococcene synthase, limonene synthase, 1,8 cineole synthase, α-pinene synthase, camphene synthase. (+)-sabinene synthase, myrcene synthase, abietadiene synthase, taxadiene synthase, farnesyl pyrophosphate synthase, amorphadiene synthase, (E)-α-bisabolene synthase, diapophytoene synthase, or diapophytoene desaturase. Additional examples of enzymes useful in the disclosed embodiments are described in Table 3.

TABLE 3

| Enzyme | Source | NCBI protein ID |
|---|---|---|
| Limonene | M. spicata | 2ONH_A |
| Cineole | S. officinalis | AAC26016 |
| Pinene | A. grandis | AAK83564 |
| Camphene | A. grandis | AAB70707 |
| Sabinene | S. officinalis | AAC26018 |
| Myrcene | A. grandis | AAB71084 |
| Abietadiene | A. grandis | Q38710 |
| Taxadiene | T. brevifolia | AAK83566 |
| FPP | G. gallus | P08836 |
| Amorphadiene | A. annua | AAF61439 |
| Bisabolene | A. grandis | O81086 |
| Diapophytoene | S. aureus | |
| Diapophytoene desaturase | S. aureus | |
| GPPS-LSU | M. spicata | AAF08793 |
| GPPS-SSU | M. spicata | AAF08792 |
| GPPS | A. thaliana | CAC16849 |
| GPPS | C. reinhardtii | EDP05515 |
| FPP | E. coli | NP_414955 |
| FPP | A. thaliana | NP_199588 |
| FPP | A. thaliana | NP_193452 |
| FPP | C. reinhardtii | EDP03194 |
| IPP isomerase | E. coli | NP_417365 |
| IPP isomerase | H. pluvialis | ABB80114 |
| Limonene | L. angustifolia | ABB73044 |
| Monoterpene | S. lycopersicum | AAX69064 |
| Terpinolene | O. basilicum | AAV63792 |
| Myrcene | O. basilicum | AAV63791 |
| Zingiberene | O. basilicum | AAV63788 |
| Myrcene | Q. ilex | CAC41012 |
| Myrcene | P. abies | AAS47696 |
| Myrcene, ocimene | A. thaliana | NP_179998 |
| Myrcene, ocimene | A. thaliana | NP_567511 |
| Sesquiterpene | Z. mays; B73 | AAS88571 |
| Sesquiterpene | A. thaliana | NP_199276 |
| Sesquiterpene | A. thaliana | NP_193064 |
| Sesquiterpene | A. thaliana | NP_193066 |
| Curcumene | P. cablin | AAS86319 |
| Farnesene | M. domestica | AAX19772 |
| Farnesene | C. sativus | AAU05951 |
| Farnesene | C. junos | AAK54279 |
| Farnesene | P. abies | AAS47697 |
| Bisabolene | P. abies | AAS47689 |
| Sesquiterpene | A. thaliana | NP_197784 |
| Sesquiterpene | A. thaliana | NP_175313 |
| GPP Chimera | | |
| GPPS-LSU + SSU fusion | | |
| Geranylgeranyl reductase | A. thaliana | NP_177587 |
| Geranylgeranyl reductase | C. reinhardtii | EDP09986 |
| Chlorophyllidohydrolase | C. reinhardtii | EDP01364 |
| Chlorophyllidohydrolase | A. thaliana | NP_564094 |
| Chlorophyllidohydrolase | A. thaliana | NP_199199 |
| Phosphatase | S. cerevisiae | AAB64930 |
| FPP A118W | G. gallus | |

The enzyme may also be β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (-)-β-pinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-1,3-ene synthase, E-β-ocimene, S-linalool synthase, geraniol synthase, γ-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase.

Biodegradative Enzymes

In some embodiments, the exogenous or endogenous polynucleotide encodes a biodegradative enzyme, which is an enzyme involved in biodegradation. For example, glucanase is an enzyme that degrades glucans, which are important structural compounds in the cell walls of plants and fungi. Glycosidases are enzymes that catalyze the hydrolysis of a glycosidic linkage to generate two smaller sugars. Glycosidases are common enzymes involved in the degradation of biomass such as cellulose and hemicellulose, in anti-bacterial defense strategies (for example, lysozyme damages bacterial cell walls), in pathogenetic mechanisms (for example, viral neuraminidases), and in normal cellular functions (for example, trimming mannosidases involved in N-linked glycoprotein biosynthesis). Examples of biodegradative enzymes that may be used in the present disclosure include, but are not limited to, exo-β-glucanase, endo-β-glucanase, β-glucosidase, endoxylanase or lignase.

Flocculating Moieties

In some embodiments, the exogenous or endogenous polynucleotide encodes a flocculating moiety. Flocculation is a process of contact and adhesion whereby the particles of a dispersion form larger-size clusters. Flocculation can be used for both large and small scale applications. Certain chemical flocculants, such as heavy metals, pose a challenge as the metal may need to be removed from the flocculated organism for downstream processes (for example, enzyme purification, nutriceutical production, or transesterification oftriglycerides) to proceed properly. A flocculation moiety can be incorporated into an organism by transforming the organism with a vector comprising a sequence encoding the flocculating moeity. Techniques involved include, but are not limited to, the development of a suitable expression cassette, insertion (i.e. transformation) of the expression cassette into a host cell, and screening of the host cell for expression of the desired flocculation moiety. Depending on the design of the vector, the flocculation moiety can be constitutively expressed (e.g., at all times) or can be inducibly expressed (e.g., temperature-induced or quorum-induced). Engineered organisms capable of expressing one or more flocculation moieties can be used for flocculation with or without the addition of other compounds. For example, one host organism (e.g., C. reinhardtii) may be transformed so as to produce the FhuA protein from E. coli and a second host organism—the same or a different species than the first organism—may be transformed so as to produce the T5 phage tail protein, pb5. FhuA and pb5 form a very stable 1:1 stoichiometric complex, thus, by combining the two transformed host cells at a desired time, or by controlling expression of the two flocculation moieties in the different strains to only express the flocculation moieties at a desired time, binding between the two moieties will cause flocculate via the interaction of the two moieties.

A flocculating moiety will typically be expressed such that it is present on the outer surface of the host cell (e.g., cell wall and/or cell membrane). In some instances, a flocculation moiety is one member of a protein binding pair. Protein pairs forming strong protein-protein complexes are useful as flocculation moieties. Self-aggregating proteins, proteins capable of forming multimeric complexes are also useful as flocculants. Carbohydrate moieties of glycoproteins (e.g., arabinosyl, galactosyl, mannosyl, and rhamnosyl residues), membrane and/or cell wall carbohydrates (e.g., alginic acid, xylanes, mannanes, agarose, carrageenan, porphyran, and furcelleran), and proteins increasing the production of certain carbohydrates or glycolipids may also be used to induce flocculation, as certain classes of carbohydrates are known components of protein complex formation.

Flocculation moieties can also be recombinantly expressed in host cells and purified to a useful level (e.g., homogeneity). The purified flocculants can be added to a target cell culture to cause flocculation. Such flocculants typically will not pose the same challenges as the use of heavy metal flocculants described above, because the flocculants should not be toxic and should not interfere with downstream processes. A recombinant lectin can be produced by a host cell (e.g. secreted or produced on the surface), collected, and introduced into a culture of an organism to be flocculated.

In one embodiment of the present disclosure, c-type lectin is expressed on the cell wall of C. reinhardtii, which induces flocculation by binding to a glycopeptide on the surface of C. reinhardtii cells. Examples of cell surface moieties include, but are not limited to, lysophosphatidic acid, c-type lectin, Gal/GalNAc, O-linked sugars, O-linked polysaccharides, GlcNAc, phospholipase A2, GalNAc-SO₄, sialic acid, glycosphingolipids, glucose monomycolate, lipoarabinomannan, phosphatidyl inositols, hexosyl-1-phosphoisoprenoids, mannosyl-phosphodolicols, α-galactosylceramide, and terminal galactosides. Examples of carbohydrate binding proteins include, but are not limited to, CD-SIGN, dectin-1, dectin-2, HECL, langerin, layilin, mincle, MMGL, E-selection, P-selectin, L-selectin, DEC-205, Endo 180, mannose receptors, phospholipase A2 receptors, sialoadhesin (siglec-1), siglec-2, siglec-3, siglec-4, siglec-5, siglec-6, siglec-7, siglec-8, siglec-9, siglec-10, siglec-11, or galectins.

Another category of proteins which may be utilized as flocculation moieties in the present disclosure are antibodies. For example, antibodies against known cell surface antigens expressed on an organism can be used. For example, C. reinhardtii may be transformed to inducibly express an anti-Fus1 antibody, which detects Fus1 protein on the external surface of fertilization tubules of C. reinhardtii. The antibodies useful for the present disclosure may be univalent, multivalent, or polyvalent. Other antibodies against various glycoproteins are known in the art (for example, as described in Matsuda et. al., J. Plant. Res., 100:373-384, 1987; and Musgrave et al., Planta, 170:328-335, 1987).

In one aspect, the present disclosure provides a method for increasing salt tolerance of an organism, for example, a eukaryotic microalga, comprising introducing an exogenous or endogenous nucleic acid sequence into the eukaryotic microalga, wherein the exogenous or endogenous nucleic acid sequence encodes an ion transporter or a protein that regulates the expression of a ion transporter, to produce a eukaryotic microalga having increased salt tolerance.

In some embodiments, the method further comprises plating the eukaryotic microalga on solid or semisolid selection media or inoculating the eukaryotic microalga into a liquid selection media, wherein the selection media comprises a concentration of salt that does not permit growth of the organism (eukaryotic microalga) not comprising the exogenous or endogenous sequence, and selecting at least one eukaryotic microalga comprising the exogenous or endogenous sequence, by the viability of the eukaryotic microalga on or in the selection media. In some embodiments, the exogenous or endogenous sequence encodes an ion transporter. For example, the ion transporter can be an ATPase, an antiporter, or an H+ pyrophosphatase.

In another aspect, the present disclosure provides a method of selecting a transformant comprising an exogenous or endogenous polynucleotide sequence encoding a protein of interest, comprising: (a) introducing a first exogenous or endogenous polynucleotide encoding a protein of interest into an alga, (b) introducing a second exogenous or endogenous polynucleotide encoding a protein of interest into the alga, wherein the second exogenous or endogenous sequence confers salt tolerance; (c) plating the alga on solid or semisolid selection media or inoculating the alga into liquid selection media, wherein the selection media comprises a concentration of salt that does not permit growth of alga not comprising the second exogenous or endogenous sequence conferring salt tolerance; and (d) selecting at least one alga comprising the second exogenous or endogenous sequence by the viability of the alga on or in the selection medium.

In some embodiments of the disclosed methods, the exogenous or endogenous sequence comprises one or more polynucleotides, which encode for one or more polypeptide(s). A polypeptide can be operatively linked to a second, third, fourth, fifth, sixth, seventh, eighth, ninth, tenth and/or subsequent polypeptide. For example, several enzymes in a hydrocarbon production pathway may be linked, either directly or indirectly, such that products produced by one enzyme in the pathway, once produced, are in close proximity to the next enzyme in the pathway.

The first and the second exogenous or endogenous polynucleotides can be on different nucleic acid molecules or on the same nucleic acid molecule or polynucleotide. In some embodiments, the second exogenous or endogenous polynucleotide encodes a transporter, a protein that regulates the expression of a transporter, a bbc protein or a functional homolog thereof, a SCSR protein or a functional homolog thereof, a chaperonin, or an antioxidant enzyme. The second exogenous or endogenous polynucleotide may encode an ion transporter, such as an ATPase, an antiporter, or a H+ pyrophosphatase.

For transformation of an alga, for example, C. reinhardtii, a nucleic acid construct which comprises both a selectable marker, e.g. salt tolerance, and one or more genes of interest can be used. In some embodiments, transformation of chloroplasts is performed by co-transformation of chloroplasts with two constructs: one containing the selectable marker and a second containing the gene(s) of interest. The gene of interest can encode a therapeutic protein, a nutritional protein, an industrial enzyme, a protein that participates in or promotes the synthesis of at least one nutritional, therapeutic, commercial, or fuel product, or a protein that facilitates the isolation of at least one nutritional, therapeutic, commercial, or fuel product. The gene of interest may also be a fuel product, a fragrance product, or an insecticide product.

In yet another aspect, the present disclosure provides a method for producing one or more biomolecules, comprising: (a) growing transgenic alga transformed with a polynucleotide encoding an ion transporter or protein that regulates the expression of an ion transporter, at a concentration of salt that inhibits the growth of non-transformed alga; and (b) harvesting one or more biomolecules from the alga.

A product or a protein of interest as disclosed herein, including, but not limited to, a therapeutic protein, a nutritional protein, an industrial enzyme, a fuel product, a fragrance product, an insecticide product, a protein that participates in or promotes the synthesis of at least one nutritional, therapeutic, commercial, fuel, fragrance, or insecticide product, or a protein that facilitates the isolation of at least one nutritional, therapeutic, commercial, fuel, fragrance, or insecticide product, may be produced by a method that comprises the steps of: growing transgenic alga transformed with a first polynucleotide encoding an ion transporter or protein that regulates the expression of an ion transporter and a second polynucleotide encoding the protein of interest, at a concentration of salt that inhibits the growth of non-transformed alga. Transformation can occur using any method known in the art or described herein. The growing/culturing step can occur in suitable medium, such as one that has minerals and/or vitamins, for example. The methods disclosed herein can further comprise the step of harvesting one or more proteins of interest from the alga. The methods described herein may further comprise the step of providing to the organism a source of inorganic carbon, such as flue gas. In some instances, the inorganic carbon source provides all of the carbon necessary for making the product (for example, a fuel product).

Also provided herein is a method for producing a product or a protein of interest that comprises: transforming an organism with an expression vector comprising a nucleic acid sequence encoding a protein of interest, growing the organism, and collecting the product or protein from the organism. Any of the vectors described herein can be used in the disclosed methods. A vector can be used to add additional biosynthetic capacity to an organism or to modify an existing biosynthetic pathway within the organism, either with the intent of increasing or allowing the production of a molecule by the organism.

Growth of Organisms

Organisms can be cultured or grown in conventional fermentation bioreactors, which include, but are not limited to, batch, fed-batch, cell recycle, and continuous fermentors. Furthermore, organisms may be cultured in photobioreactors (for example, as described in U.S. Appl. Publ. No. 2005/0260553; U.S. Pat. No. 5,958,761; and U.S. Pat. No. 6,083,740). Culturing or growing can also be conducted in shaker flasks, test tubes, microtiter dishes, and petri plates. Culturing is carried out at a temperature, pH, and oxygen content appropriate for the recombinant cell. Determining the proper culturing conditions are well within the expertise of one of ordinary skill in the art.

A host organism may be grown in outdoor open water, such as ponds, the ocean, sea, rivers, waterbeds, marsh water, shallow pools, lakes, reservoirs, for example. When grown in water, the organisms can be contained in a halo like object comprising of lego-like particles. The halo object encircles the algae and allows it to retain nutrients from the water beneath while keeping it in open sunlight.

In some instances, organisms can be grown in containers wherein each container comprises 1 or 2 or a plurality of organisms. The containers can be configured to float on water. For example, a container can be filled by a combination of air and water to make the container and the host organism(s) in it buoyant. A host organism that is adapted to grow in fresh water can thus be grown in salt water (for example, the ocean) and vice versa. This mechanism allows for automatic death of the organism if there is any damage to the container.

In some instances a plurality of containers can be contained within a halo-like structure as described above. For example, up to 100, 1,000, 10,000, 100,000, or 1,000,000 containers can be arranged in a meter-square of a halo-like structure.

An organism, for example, a host algae transformed to produce a protein described herein, for example, a transporter, can be grown on land, e.g., ponds, aqueducts, landfills, or in closed or partially closed bioreactor systems. Algae can also be grown directly in water, for example, in oceans, seas, lakes, rivers, and reservoirs. In embodiments where algae are mass cultured, the algae can be grown in high-density photobioreactors. Methods of mass culturing algae are known in the art. For example, algae can be grown in high density photobioreactors (for example, as described in Lee et al, Biotech. Bioengineering 44:1161-1167, 1994) and other bioreactors (such as those for sewage and waste water treatments) (for example, as described in Sawayama et al, Appl. Micro. Biotech., 41:729-731, 1994). Additionally, algae may be mass cultured to remove heavy metals (for example, as described in Wilkinson, Biotech. Letters, 11:861-864, 1989), hydrogen (for example, as described in U.S. Patent Application Publication No. 20030162273), and pharmaceutical compounds.

The photosynthetic organism (e.g. genetically modified algae) can be grown under any suitable condition, for example under conditions which permit photosynthesis or in the absence of light.

In some embodiments, the product or protein of interest (for example a therapeutic protein, nutritional protein, industrial enzyme, fuel product, fragrance product, or insecticide product) is collected by harvesting the organism. The product may then be extracted from the organism.

The product-containing biomass can be harvested from its growth environment (e.g. lake, pond, photobioreactor, or partially closed bioreactor system) using any suitable method. Non-limiting examples of harvesting techniques are centrifugation or flocculation. Once harvested, the product-containing biomass can be subjected to a drying process. Alternately, an extraction step may be performed on wet biomass. The product-containing biomass can be dried using any suitable method. Non-limiting examples of drying methods include sunlight, rotary dryers, flash dryers, vacuum dryers, ovens, freeze dryers, hot air dryers, microwave dryers and superheated steam dryers. After the drying process the product-containing biomass can be referred to as a dry or semi-dry biomass. The moisture content of the dry or semi-dry biomass can be up to about 20%, about 15%, about 10%, about 5%, about 4%, about 3%, about 2% or about 1% (wt/wt).

The following examples are intended to provide illustrations of the application of the present invention. The following examples are not intended to completely define or otherwise limit the scope of the invention.

### Example 1

### Nuclear Transformation of C. reinhardtii with a Gene that Confers Salt Tolerance

In this example a polynucleotide encoding NHX1 protein is introduced into C. reinhardtii. The plasmid construct contains the gene encoding NHX1 regulated by the 5′ UTR and promoter sequence for the HSP70A/rbcS2 gene from C. reinhardtii, and the 3′ UTR sequence for the rbcS2 gene from C. reinhardtii. The hygromycin resistance gene is expressed as a selectable marker. The hygromycin resistance gene and NHX1 coding regions are physically linked in-frame, resulting in a chimeric single open reading frame (ORF). A Metal Affinity Tag (MAT) and FLAG epitope tag are added to the 3′ end of the ORF, using standard techniques. The transgene cassette is flanked by segments of an appropriate nuclear genomic locus of C. reinhardtii for genomic integration of the transgene via homologous recombination. Electroporation, which is a known technique in the art, is used for nuclear transformation. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations are carried out on C. reinhardtii strain CC1690 (mt+). Cells that have been successfully transformed with the NHX1 gene are tolerant to higher concentrations of salt. Cells are grown to late log phase (approximately 7 days) in the presence of 500 mM NaCl in TAP medium (as described in Gorman and Levine, Proc. Natl. Acad. Sci., USA 54:1665-1669, 1965, which is incorporated herein by reference) at 23° C. under constant illumination of 450 Lux on a rotary shaker set at 100 rpm. All transformations are carried out under high salt selection (greater than 200 mM) in which salt resistance is conferred by the presence of the NHX1 gene.

PCR is used to identify transformed strains. For PCR analysis, 10⁶ algae cells (from agar plate or liquid culture) are suspended in 10 mM EDTA and heated to 95° C. for 10 minutes, then cooled to near 23° C. A PCR cocktail consisting of reaction buffer, MgCl₂, dNTPs, PCR primer pair(s), DNA polymerase, and water is prepared. Algae lysate in EDTA is added to provide a template for the reaction. The magnesium concentration is varied to compensate for the amount and concentration of algae lysate in EDTA added. Annealing temperature gradients are employed to determine optimal annealing temperature for specific primer pairs.

To identify strains that contain the NHX1 gene, a primer pair is used in which one primer anneals to a site within the rbcS2 5′UTR and the other primer anneals within the NHX1 coding segment. Desired clones are those that yield a PCR product of expected size. Cultivation of C. reinhardtii transformants for expression of NHX1 is carried out in liquid TAP medium containing 500 mM NaCl at 23° C. in the dark on a rotary shaker set at 100 rpm, unless stated otherwise. Cultures are maintained at a density of 1×10⁷ cells per ml for at least 48 hr prior to harvest.

To determine if the NHX11 gene leads to expression of the NHX1 protein in transformed algae cells, soluble proteins are immunopreciptated and visualized by Western blot. Briefly, 500 mls of algae cell culture is harvested by centrifugation at 4000×g at 4° C. for 15 min. The supernatant is decanted and the cells are resuspended in 10 mls of lysis buffer (100 mM Tris-HCl, pH=8.0, 300 mM NaCl, 2% Tween-20). Cells were lysed by sonication (10×30 sec at 35% power). Lysate is clarified by centrifugation at 14,000×g at 4° C. for 1 hour. The supernatant is removed and incubated with anti-FLAG antibody-conjugated agarose resin at 4° C. for 10 hours. Resin is separated from the lysate by gravity filtration and washed 3× with wash buffer (100 mM Tris-HCl, pH=8.0, 300 mM NaCl, 2% Tween-20). Results from Western blot analysis show that NHX1 protein is produced.

### Example 2

### Nuclear Transformation of C. reinhardtii with Limonene Synthase Gene

In this example, C. reinhardtii is transformed with a first exogenous polynucleotide encoding an ENA1 protein and a second exogenous polynucleotide encoding a limonene synthase. The gene encoding limonene synthase and the gene encoding ENA1 that confers salt tolerance to the transformed algal cells are both regulated by the 5′ UTR and promoter sequence for the HSP70A/rbcS2 gene from C. reinhardtii and the 3′ UTR sequence of the rbcS2 gene from C. reinhardtii. The transgene cassette is flanked by segments of an appropriate nuclear genomic locus of C. reinhardtii for genomic integration of the transgene via homologous recombination. Electroporation, which is a known technique in the art, is used for nuclear transformation. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations are carried out on C. reinhardtii strain CC1690 (mt+). Cells are grown to late log phase (approximately 7 days) in the presence of 3 mM lithium salt in TAP medium (as described in Gorman and Levine, Proc. Natl. Acad. Sci., USA 54:1665-1669, 1965, which is incorporated herein by reference) at 23° C. under constant illumination of 450 Lux on a rotary shaker set at 100 rpm. All transformants are selected under high salt concentration (3 mM Li+ salt) to which resistance is conferred by the presence of the ENA1 gene.

PCR is used to identify transformed strains. For PCR analysis, 10⁶ algae cells (from agar plate or liquid culture) are suspended in 10 mM EDTA and heated to 95° C. for 10 minutes, then cooled to near 23° C. A PCR cocktail consisting of reaction buffer, MgCl₂, dNTPs, PCR primer pair(s), DNA polymerase, and water is prepared. Algae lysate in EDTA is added to provide a template for the reaction. The magnesium concentration is varied to compensate for the amount and concentration of algae lysate in EDTA added. Annealing temperature gradients are employed to determine optimal annealing temperature for specific primer pairs.

To identify strains that contain the limonene synthase gene, a primer pair is used in which one primer anneals to a site within the rbcS2 5′UTR and the other primer anneals within the limonene synthase coding segment. Desired clones are those that yield a PCR product of expected size.

Cultivation of C. reinhardtii transformants for expression of limonene synthase is carried out in liquid TAP medium containing 3 mM lithium salt at 23° C. in the dark on a rotary shaker set at 100 rpm, unless stated otherwise. Cultures are maintained at a density of 1×10⁷ cells per ml for at least 48 hr prior to harvest.

To determine if the limonene synthase gene leads to expression of the limonene synthase in transformed algae cells, both soluble proteins are immunopreciptated and visualized by Western blot. Briefly, 500 mls of algae cell culture is harvested by centrifugation at 4000×g at 4° C. for 15 min. The supernatant is decanted and the cells resuspended in 10 mls of lysis buffer (100 mM Tris-HCl, pH=8.0, 300 mM NaCl, 2% Tween-20). Cells are lysed by sonication (10×30 sec at 35% power). Lysate is clarified by centrifugation at 14,000×g at 4° C. for 1 hour. The supernatant is removed and incubated with anti-FLAG antibody-conjugated agarose resin at 4° C. for 10 hours. Resin is separated from the lysate by gravity filtration and washed 3× with wash buffer (100 mM Tris-HCl, pH=8.0, 300 mM NaCl, 2% Tween-20). Results from Western blot analysis show that limonene synthase is produced.

To determine whether limonene synthase produced is a functional enzyme, limonene production from GPP (geranyl diphosphate) is examined. Briefly, 50 uls of the limonene synthase-bound agarose (same samples prepared above) is suspended in 300 uls of reaction buffer (25 mM HEPES, pH=7.2, 100 mM KCl, 10 mM MnCl2, 10% glycerol, and 5 mM DTT) with 0.33 mg/mls GPP and transferred to a glass vial. The reaction is overlaid with heptane and incubated at 23° C. for 12 hours. The reaction is quenched and extracted by vortexing the mixture. 0.1 mls of heptane is removed and the sample is analyzed by GC-MS.

Limonene synthase activity from crude cell lysates is also examined. Briefly, 50 mls of algae cell culture is harvested by centrifugation at 4000×g at 4° C. for 15 min. The supernatant is decanted and the cells are resuspended in 0.5 mls of reaction buffer (25 mM HEPES, pH=7.2, 100 mM KCl, 10 mM MnCl₂, 10% glycerol, and 5 mM DTT). Cells are lysed by sonication (10×30 sec at 35% power). 0.33 mg/mls of GPP is added to the lysate and the mixture is transferred to a glass vial. The reaction is overlaid with heptane and incubated at 23° C. for 12 hours. The reaction is quenched and extracted by vortexing the mixture. 0.1 mls of heptane is removed and the sample is analyzed by GC-MS.

### Example 3

### Production of a Flocculation Moiety by C. reinhardtii

In this example, a nucleic acid encoding a C-type lectin with a fused secretion signal under control of a quorum-sensing promoter is introduced into C. reinhardtii along with the SOS1 gene that confers salt tolerance in the transformed C. reinhardtii. The construct contains the C-type lectin encoding gene under control of the 5′ UTR and promoter sequence for the HSP70A/rbcS2 gene from C. reinhardtii and the 3′ UTR for the rbcS2 gene from C. reinhardtii. The construct also contains the SOS1 gene, which is regulated by the 5′ UTR and promoter sequence for the HSP70/rbcS2 gene from C. reinhardtii and the 3′ UTR sequence for the rbcS2 gene from C. reinhardtii. The zeocin resistance gene is expressed separately driven by a beta-2 tubulin promoter. The transgene cassette is flanked by segments of an appropriate nuclear genomic locus of C. reinhardtii for genomic integration of the transgene via homologous recombination. Electroporation, which is a known technique in the art, is used for nuclear transformation. All DNA manipulations carried out in the construction of this transforming DNA are essentially as described by Sambrook et al., Molecular Cloning: A Laboratory Manual (as described in Cold Spring Harbor Laboratory Press 1989) and Cohen et al., Meth. Enzymol. 297, 192-208, 1998.

For these experiments, all transformations are carried out on C. reinhardtii strain CC1690 (mt+). Cells are grown to late log phase (approximately 7 days) in the presence of 300 mM NaCl in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. All transformations are carried out under high salt selection (300 mM NaCl), in which resistance is conferred by the SOS1 gene.

PCR is used to identify transformed strains as described in details in Examples 1 and 2. The transformed algae possess salt tolerance and produce the c-type lectin.

To produce the c-type lectin, cells of this strain are grown under standard conditions. Upon reaching appropriate cell density (2×10⁶), expression of C-type lectin is induced. The C-type lectin binds to one or more cell surface carbohydrates and/or glycoproteins, resulting in a gradual and increasing flocculation of the culture. Flocculation occurs, presumably, due to the binding of the recombinant C-type lectin flocculation moiety with one or more naturally occurring flocculation moieties present on the surface of the cells.

After flocculation, one or more products are collected from the flocculated cells and/or the liquid environment. The cells are ground and the cell wall portion is separated using an affinity column which binds to the recombinant C-type lectin. The isolated lectin can then be added to another culture expressing a C-type lectin-compatible flocculation moiety to induce further flocculation.

### Example 4

### Selecting Gene Targets for Stress Resistance

Eight genes (SR1-SR8) were chosen based on the ability of the various genes to provide salt resistance to other organisms, other than photosynthetic microorganisms. SR1-SR8 are either the protein that was described or a homolog of the protein that was described.

GPX (SR1), BBC1 (SR7), CW80Cd404 (SR8) were all identified as Chlamydomonas genes conferring salt tolerance to E. coli by Miyasaka, et al. (2000) World Journal of Microbiology and Biotechnology, Vol 16:23-29. Also, BBC1 (SR7) was further analysed by Tanaka, et al. (2001) Curr Micro 42, 173-177. SR2 and SR3 are homologs of SR1. NHX1 from Arabidposis thaliana (SR5) was described by Tian, et al. (2006) African Journal of Biotechnology, Vol. 5, Issue 11, pp. 1041-1044; Apse, M. P., et al. (1999) Salt tolerance conferred by overexpression of a vacuolar NaC/HC antiport in Arabidopsis, Science 285, 1256-1258; Zhang, H. X. and Blumwald, E. (2001) Transgenic salt-tolerant tomato plants accumulate salt in foliage but not in fruit, Nat. Biotechnol. 19, 765-768; Zhang, H. X. et al. (2001) Engineering salt-tolerant Brassica plants: characterization of yield and seed oil quality in transgenic plants with increased vacuolar sodium accumulation, Proc. Natl. Acad. Sci. U.S.A. 98. 12832-12836; Yin, X. Y. et al. (2004) Production and analysis of transgenic maize with improved salt tolerance by the introduction of AtNHX1 gene, Acta Bot. Sin. 46, 854-861; and Xue, Z. Y. et al. (2004) Enhanced salt tolerance of transgenic wheat (Tritivum aestivum L.) expressing a vacuolar Na+/H+ antiporter gene with improved grain yields in saline soils in the field and a reduced level of leaf Na+. Plant Sci. 167, 849-859. A. gmelini NHX1 (SR4) was described in Ohta, M. et al. (2002) Introduction of a Na+/H+ antiporter gene from Atriplex gmelini confers salt tolerance in rice, FEBS Lett. 532, 279-282. Arabidopsis SOS1 (SR6) was described by Shi, H. et al. (2003) Overexpression of a plasma membrane Na+/H+ antiporter gene improves salt tolerance in Arabidopsis thaliana, Nat. Biotechnol. 21, 81-85.

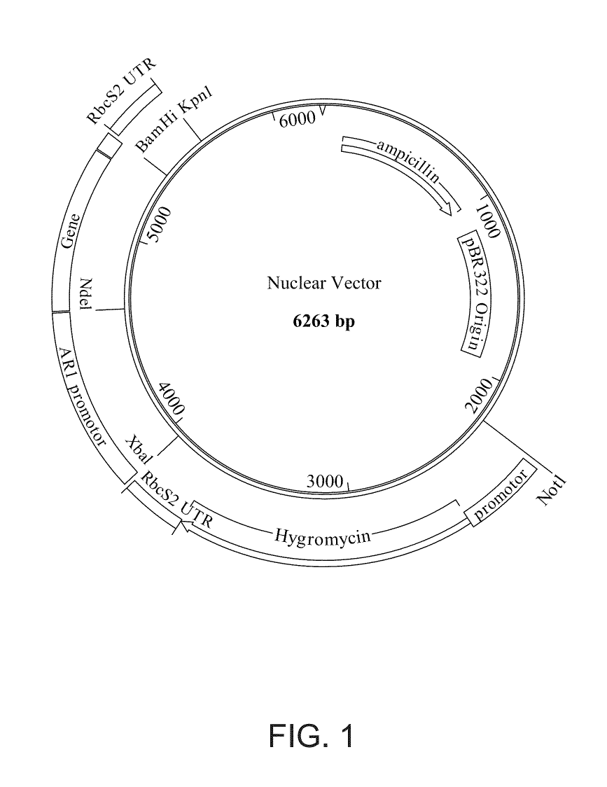

All of the 8 sequences were synthesized by DNA2.0 (Menlo Park, Calif., USA) and were codon optimized to reflect the codon usage in the nuclear genome of Chlamydomonas reinhardtii. The nucleic acid sequences encoding the SR1-SR8 genes were each individually cloned into the nuclear vector shown in FIG. 1, between the NdeI site and the BamHI site. The following nucleic acid sequences were individually cloned into the nuclear vector; SEQ ID NO: 24, SEQ ID NO: 29, SEQ ID NO: 33, SEQ ID NO: 37, SEQ ID NO: 41, SEQ ID NO: 45, SEQ ID NO: 49, and SEQ ID NO: 53.

Nuclear transformations were carried out for all eight genes as described in the Example 5 to Example 12 below. The transformants were either selected for on media containing Hygromycin (20 ug/ml) to select for integration of the nuclear vector into the host nuclear genome, or media containing both Hygromycin and salt selection to select for expression of the SR gene.

Transformants were grown to saturation in 200 ul liquid cultures in 96-well plates. Transformants were then subcultured into 200 ul liquid cultures in 96-well plates containing varying levels of salt. In TAP media, the concentrations of added NaCl used were 100 mM, 200 mM, 250 mM, and 300 mM. In greenhouse (G) media buffered with 50 mM CHESS pH 9.0, the concentrations of NaCl used were 50 mM, 75 mM, and 100 mM.

Transformants that grew in the presence of salt were scaled up for growth in 6-well plates to confirm the phenotype. Transformants that continued to show the salt tolerance phenotype from the 6-well plates were scaled up for growth curves in 50 ml flasks. 50 ml cultures were diluted to identical cell densities in media containing various levels of added NaCl. In TAP media, the concentrations of added NaCl used were 0 mM, 100 mM, 200 mM, 250 mM, and 300 mM. In G media buffered with 50 mM CHESS pH 9.0, the concentrations of added NaCl used were 0 mM, 50 mM, 75 mM, and 100 mM. The growth of the transformants were measured by cell density of the culture over time (for example, up to 14 days). A transformant was determined to be salt resistant if it showed a faster growth rate than an untransformed alga in the presence of salt.

To confirm that the salt resistant phenotype is due to the expression of the SR gene, a Western blot is performed to detect the presence of an SR protein. An additional means of confirming the expression of the SR gene is by PCR and RTPCR to show transcription of the gene. A method of linking the presence of the gene to the salt resistant phenotype is by mating transformants with the untransformed alga. Any progeny that do not contain the SR gene should not be salt tolerant, while progeny that contain the SR gene should be salt tolerant. One skilled in the art would realize that segregation of progeny is not always 100%.

The results from the transformations are shown below in Table 4. The numbers below are taken from four separate transformations and reflect transformants grown in both TAP and G media as described above.

TABLE 4

| | Number of | Number of | Number of |
|---|---|---|---|
| | transformants | transformants | transformants that |
| | screened in 96-well | screened in 6-well | showed salt resistance |
| SR gene | plates. | plates. | in 6-well plates. |
| SR1 | 384 | 14 | 4 |
| SR2 | 207 | 22 | 2 |
| SR3 | 204 | 17 | 4 |
| SR4 | 198 | 13 | 3 |
| SR5 | 8 | 0 | 0 |
| SR6 | 0 | 0 | 0 |
| SR7 | 202 | 28 | 1 |
| SR8 | 271 | 25 | 6 |

### Example 5

### Nuclear Transformation of C. reinhardtii with a SR8 Gene that Confers Salt Tolerance

In this example a polynucleotide (SEQ ID NO: 53) encoding SR8 protein (SEQ ID NO: 54) was introduced into C. reinhardtii (CC1690). The plasmid construct (as shown in FIG. 1) contained the gene encoding SR8 that is regulated by the 5′ UTR and promoter sequence for the HSP70A/rbcS2 gene from C. reinhardtii and the 3′ UTR sequence for the rbcS2 gene from C. reinhardtii. A Metal Affinity Tag (MAT), a protease cleavage site (TEV) and FLAG epitope tag were added to the 3′ end of the ORF, using standard techniques. The amino acid sequence of the FLAG-TEV-MAT tag is shown in SEQ ID NO: 28. The same plasmid construct contained the hygromycin resistance gene expressed as a selectable marker regulated by the beta-Tubulin promoter and 5′UTR and rbcs2 3′UTR from C. reinhardtii. The transgene cassette can be flanked by segments of an appropriate nuclear genomic locus of C. reinhardtii for genomic integration of the transgene via homologous recombination if desired. Electroporation, which is a known technique in the art, was used for nuclear transformation. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

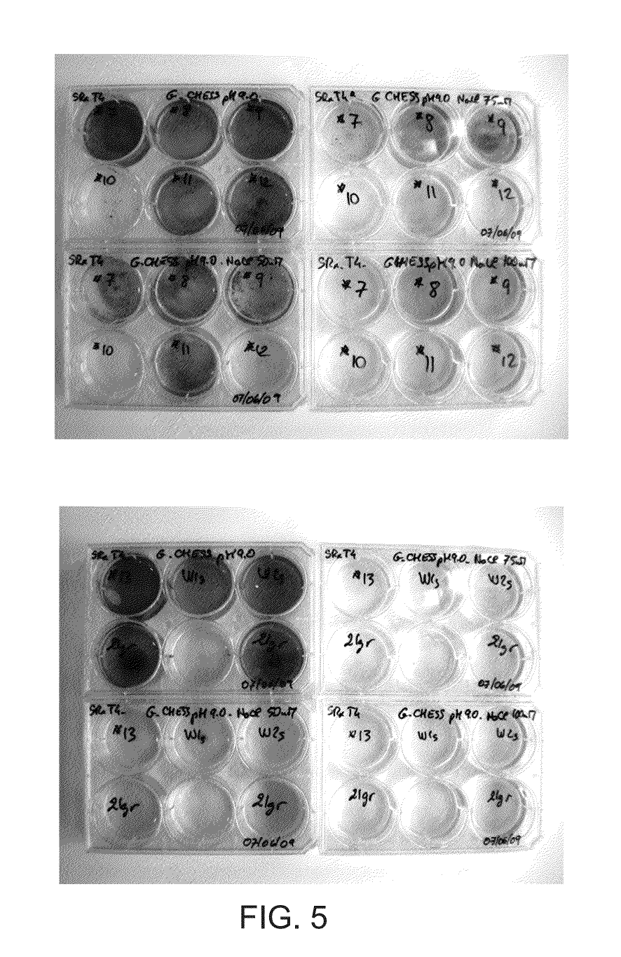

For these experiments, all transformations were carried out on C. reinhardtii strain CC1690 (mt+). Cells were grown to mid log phase in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Cells were harvested by centrifugation at 4,000×g at 4° C. for 10 min. The supernatant was decanted and cells were resuspended in TAP medium containing 40 mM Sucrose to a final concentration of 3×10̂8 cells/ml for subsequent transformation by electroporation. DNA for use in transformation was first linearized by restriction digest using an enzyme that only has one recognition site within the plasmid construct. DNA for transformation was added to 250 ul cells and placed in an 0.4 cm electroporation cuvette on ice. Conditions for electroporation were 800V, 25 uF, infinite resistance using exponential decay electroporation on a BIORAD gene pulser electroporator. Cells that were successfully transformed with SR8 gene were tolerant to higher concentrations of salt. Transformants were either selected for on media containing Hygromycin (20 ug/ml) or media containing both Hygromycin and salt selection sufficient to prevent growth of the parental strain (greater than 200 mM for TAP media). Strains that grew under these initial selection conditions were grown to saturation in 200 ul liquid cultures in 96-well format in TAP media. These cultures were further tested for salt tolerance by subculture into G media buffered with CHESS at pH9.0 containing varying amounts of salt (0, 50, 75, 100 mM added NaCl). Cultures that grew in the presence of salt were scaled up for growth in 6-well plates to confirm the phenotype, and an exemplary screen is shown in FIG. 5. Both the top and bottom panel show four 6-well plates. The upper left plate shows cultures grown in G media buffered with CHESS at pH9.0 containing 0 mM added NaCl. The lower left plate shows cultures grown in G media buffered with CHESS at pH9.0 containing 50 mM added NaCl. The upper right plate shows cultures grown in G media buffered with CHESS at pH9.0 containing 75 mM added NaCl. The lower right plate shows cultures grown in G media buffered with CHESS at pH9.0 containing 100 mM added NaCl. Dark media indicates growth of the algae and clear media indicates no growth. Top panel: the top row of each of the four plates contain cultures of algae transformed with SR8, showing growth in media containing up to at least 100 mM added NaCl. Lower panel: the lower row of each of the four plates, containing the marking “21 gr” contain cultures of the untransformed algae, and do not show growth in media containing greater than 50 mM added NaCl.

Strains that show high salt tolerance (growth in higher levels of salt than the wild type strain) were chosen for further analysis.

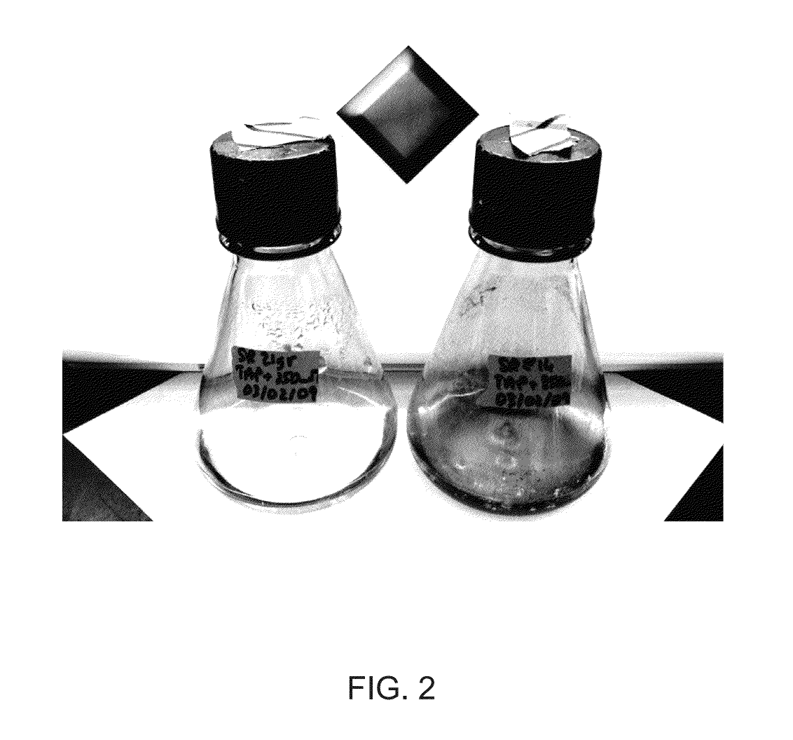

An example of one of the transformants that was further analyzed is shown in FIG. 2. FIG. 2 shows two flasks, one of untransformed Chlamydomonas (left) and the other of Chlamydomonas transformed with the SR8 gene (right). Both cultures are grown in TAP media plus 250 mM added NaCl. The untransformed culture is inhibited for growth (media remains transparent) while the culture containing SR8 is able to survive (media becomes dark with algal growth). The two cultures were grown for approximately 10 days.

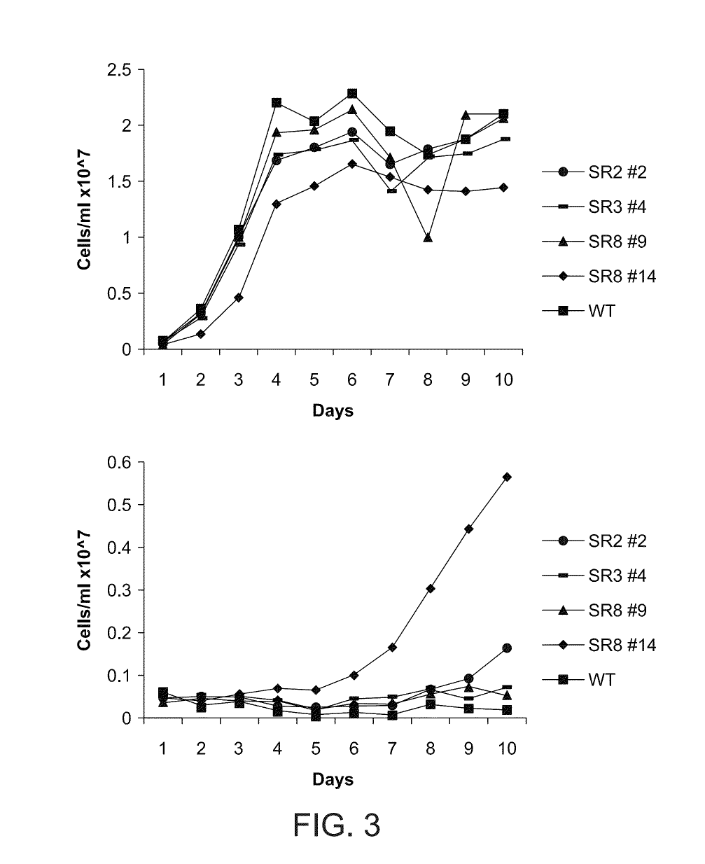

FIG. 3A shows quantitative analysis of the growth rate of transformed algae and control untransformed algae in the absence of salt (TAP media). Both transformed algae and untransformed control algae all grow in the absence of salt and show a similar growth rate. FIG. 3B shows quantitative analysis of the growth rate of transformed algae and control untransformed algae in the presence of salt (TAP plus 250 mM NaCl). Algae were grown in TAP or TAP plus 250 mM NaCl and cell density measured over time. The graphs show cell density (cells/ml×10⁷) versus time (days). SR8 (#14)(diamond) shows a higher growth rate when compared to the control untransformed algae (WT)(square).

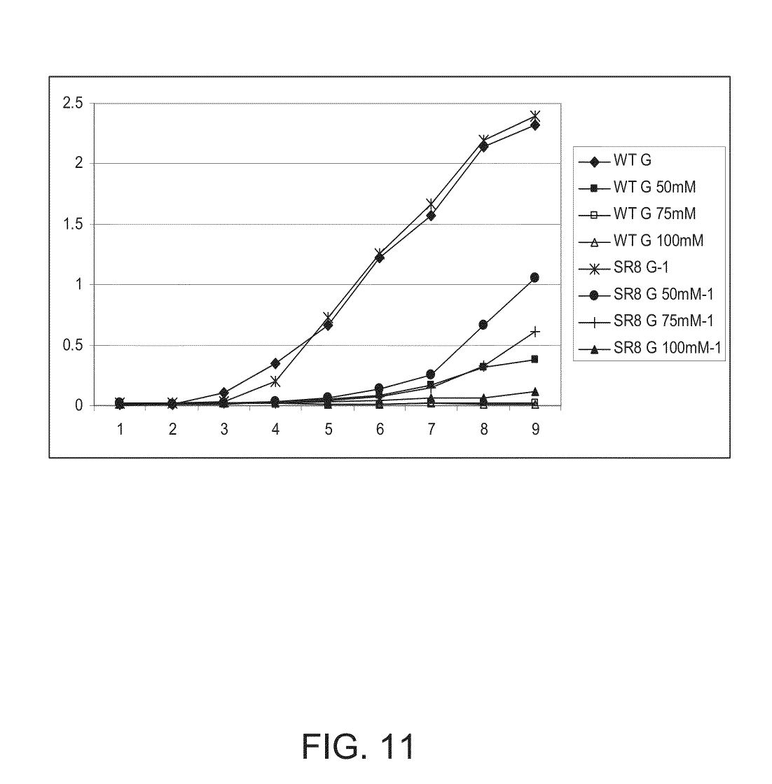

FIG. 11 shows quantitative analysis of the growth rate of transformed algae and control untransformed algae in the absence or presence of salt (grown in G media). Both transformed algae and untransformed control algae are grow in the absence of salt and show a similar growth rate (untransformed, filled diamond; SR8, star). Algae were grown in G or G plus 50 mM, 75 mM, or 100 mM NaCl and cell density measured over time. The graphs show cell density 750 nm (OD) versus time (days). SR8 (filled circle) shows a higher growth rate when compared to the control untransformed algae (WT)(filled square) when grown in G media plus 50 mM NaCl. SR8 (cross) shows a higher growth rate when compared to the control untransformed algae (WT)(open square) when grown in G media plus 75 mM NaCl. Both SR8 (filled triangle) and untransformed algae (open triangle) show a lack of growth in G media plus 100 mM NaCl.

PCR was used to identify transformed strains. For PCR analysis, 10⁶ algae cells (from agar plate or liquid culture) were suspended in 10 mM EDTA and heated to 95° C. for 10 minutes, then cooled to near 23° C. A PCR cocktail consisting of reaction buffer, MgCl₂, DMSO, Betaine, dNTPs, PCR primer pair(s), DNA polymerase, and water was prepared. Algae lysate in EDTA was added to provide template for reaction. Magnesium concentration was varied to compensate for amount and concentration of algae lysate in EDTA added. Annealing temperature gradients were employed to determine optimal annealing temperature for specific primer pairs.

To identify strains that contained the SR8 gene, a primer pair was used in which one primer annealed to a site within the rbcS2 5′UTR and the other primer annealed within the SR8 coding segment. Desired clones were those that yielded a PCR product of expected size.

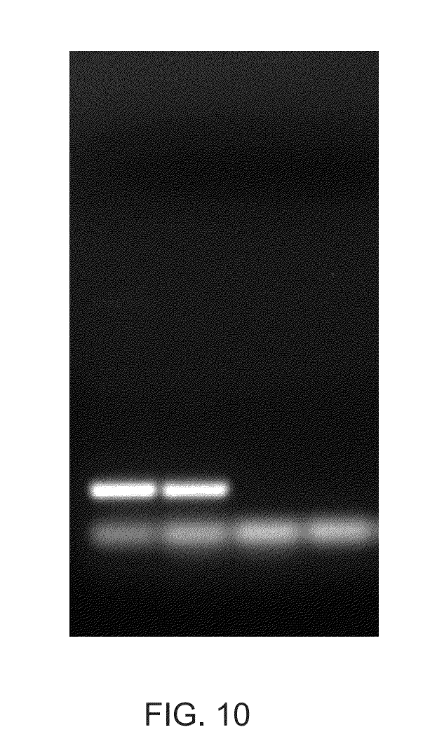

The left two columns of FIG. 10 show PCR product using algae transformed with SR8 as template. Both columns show a band of the expected size for SR8 gene. The right two columns of FIG. 10 show PCR product using untransformed algae as template. No band is seen in the right two columns.

Cultivation of C. reinhardtii transformants for expression of SR8 was carried out in liquid TAP medium containing or not containing 150 mM added NaCl at 23° C. in the light on a rotary shaker set at 100 rpm, unless stated otherwise. Cultures were grown to a cell density of 1×10⁷ cells per ml prior to harvest.

To determine if the SR8 gene led to expression of the SR8 protein in transformed algae cells, soluble proteins are immunopreciptated and visualized by Western blot. Briefly, 500 ml of algae cell culture are harvested by centrifugation at 4000×g at 4° C. for 15 min. The supernatant is decanted and the cells are resuspended in 10 ml of lysis buffer (100 mM Tris-HCl, pH=8.0, 300 mM NaCl, 2% Tween-20). Cells are lysed by sonication (10×30 sec at 35% power). Lysate is clarified by centrifugation at 14,000×g at 4° C. for 1 hour. The supernatant is removed and incubated with anti-FLAG antibody-conjugated agarose resin at 4° C. for 10 hours. Resin is separated from the lysate by gravity filtration and washed 3 times with wash buffer (100 mM Tris-HCl, pH=8.0, 300 mM NaCl, 2% Tween-20). Immunoprecipitated proteins are eluted from the resin and separated by SDS-PAGE and then transferred to PVDF for detection by Western blot.

To determine if the SR8 gene is expressed, RNA for SR8 is detected by Reverse Transcriptase PCR (RT-PCR). Total RNA is isolated from 50 ml of a saturated culture. Cells are harvested by centrifugation and frozen after removal of the supernatant. Frozen cells are resuspended in Concert Plant RNA reagent (Invitrogen) before cell lysis (by bead beating). Lysate is clarified by centrifugation at 12,000×g at 4° C. for 2 minutes. RNA is isolated from the cleared lysate by chloroform extraction and ethanol precipitation. RNA is further purified using a Qiagen Rneasy Mini Kit. DNA contamination is removed by digestion with DNAse enzyme. cDNA is generated using the BIORAD iScript cDNA synthesis kit. cDNA corresponding to the SR8 gene is detected by PCR using primers specific to the SR8 gene.

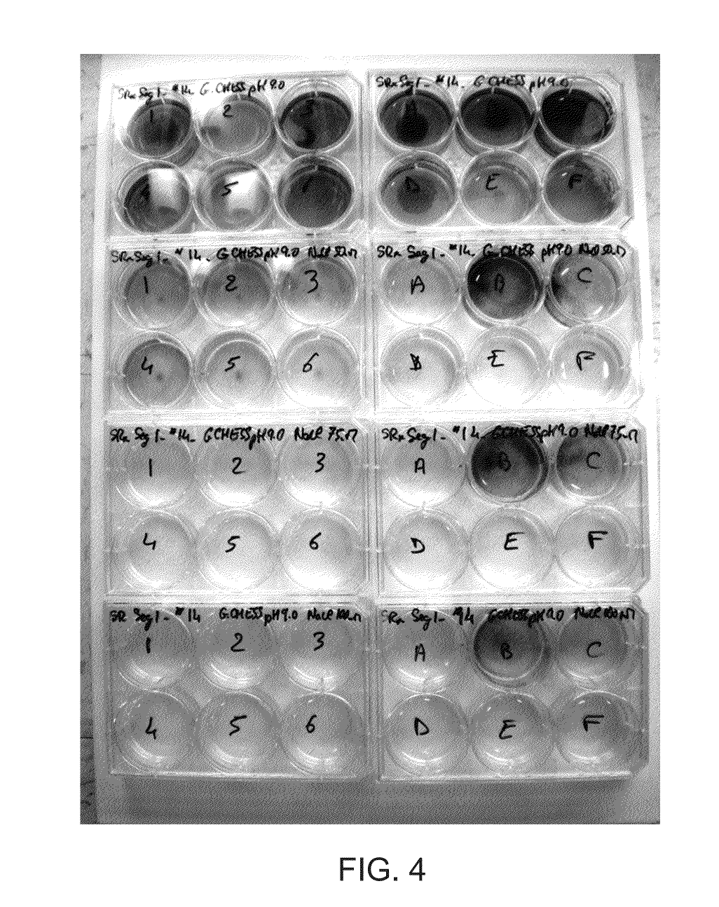

As a further piece of evidence that expression of SR8 leads to the phenotype of salt tolerance, strains containing the SR8 gene were back crossed/mated with a wild type Chlamydomonas strain of the opposite mating type (Chlamydomonas strain CC1691, mt−). Cultures to be mated were grown to early log phase, harvested by centrifugation (4000×g, 10 minutes) and resuspended in media containing no nitrogen source (HSM-NH4) to an equal volume as the original culture. Cultures were then placed at 23° C. under constant illumination of 450 Lux on a rotary shaker set at 100 rpm for 8-16 hours to induce gamete formation. Equal volumes of the mating cultures were mixed and allowed to grow for a further 16-24 hours. Cells from this culture were plated on solid media (HSM-NH4) and placed in light for 5 days. Unmated gametes were killed by chloroform treatment. Plates were placed face down above a chloroform source for 40 seconds. Cells were then cultured in liquid TAP media for 3-5 days before plating on solid media to isolate single colonies. Strains (progeny) that grew were tested for hygromycin resistance. Six strains (progeny) showing hygromycin resistance (thus should contain the SR8 gene and thus should be salt tolerant) and six strains (progeny) showing hygromycin sensitivity (thus should not contain the SR8 gene) were screened for salt tolerance. Strains (progeny) were grown to saturation in liquid media and then diluted in G media buffered with CHESS buffer at pH 9.0 containing 0, 50, 75, 100 mM added NaCl. FIG. 4 shows the results of the mating. The top two rows of cultures were grown in G media buffered with CHESS buffer at pH 9.0 with 0 mM added NaCl; the next two rows of cultures were grown in G media buffered with CHESS buffer at pH 9.0 with 50 mM added NaCl; the next two rows of cultures were grown in G media buffered with CHESS buffer at pH 9.0 with 75 mM added NaCl; and the last two rows of cultures were grown in G media buffered with CHESS buffer at pH 9.0 with 100 mM added NaCl. Dark media indicates growth of the algae and clear media indicates no growth. The left hand three columns show cultures of progeny that were sensitive to hygromycin. The right hand three columns show cultures of progeny that were resistant to hygromycin. All progeny that were sensitive to hygromycin were unable to grow in media containing added NaCl at a concentration above 50 mM. Some progeny that were resistant to hygromycin were also able to grow in media containing added NaCl at a concentration up to at least 100 mM, indicating that the salt resistant phenotype may be the result of the expression of the SR8 gene in the transformed algae.

### Example 6

### Nuclear Transformation of C. reinhardtii with a SR1 Gene that Confers Salt Tolerance

In this example a polynucleotide (SEQ ID NO: 24) encoding SR1 protein (SEQ ID NO: 25) was introduced into C. reinhardtii (CC1690). The plasmid construct (as shown in FIG. 1) contained the gene encoding SR1 that is regulated by the 5′ UTR and promoter sequence for the HSP70A/rbcS2 gene from C. reinhardtii and the 3′ UTR sequence for the rbcS2 gene from C. reinhardtii. A Metal Affinity Tag (MAT), a protease cleavage site (TEV) and FLAG epitope tag were added to the 3′ end of the ORF, using standard techniques. The amino acid sequence of the FLAG-TEV-MAT tag is shown in SEQ ID NO: 28. The same plasmid construct contained the hygromycin resistance gene expressed as a selectable marker regulated by the beta-Tubulin promoter and 5′UTR and rbcs2 3′UTR from C. reinhardtii. The transgene cassette can be flanked by segments of an appropriate nuclear genomic locus of C. reinhardtii for genomic integration of the transgene via homologous recombination if desired. Electroporation, which is a known technique in the art, was used for nuclear transformation. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain CC1690 (mt+). Cells were grown to mid log phase in TAP medium (Gorman and Levine, Proc. Natl. Acad. Sci., USA 54:1665-1669. 1965, which is incorporated herein by reference) at 23° C. under constant illumination of 450 Lux on a rotary shaker set at 100 rpm. Cells were harvested by centrifugation at 4,000×g at 4° C. for 10 min. The supernatant was decanted and cells were resuspended in TAP medium containing 40 mM Sucrose to a final concentration of 3×10̂8 cells/ml for subsequent transformation by electroporation. DNA for use in transformation was first linearized by restriction digest using an enzyme that only has one recognition site within the plasmid construct. DNA for transformation was added to 250 ul cells and placed in an 0.4 cm electroporation cuvette on ice. Conditions for electroporation were 800V, 25 uF, infinite resistance using exponential decay electroporation on a BIORAD gene pulser electroporator. Cells that were successfully transformed with SR8 gene were tolerant to higher concentrations of salt. Transformants were either selected for on media containing Hygromycin (20 ug/ml) or media containing both Hygromycin and salt selection sufficient to prevent growth of the parental strain (greater than 200 mM for TAP media). Strains that grew under these initial selection conditions were grown to saturation in 200 ul liquid cultures in 96-well format in TAP media. These cultures were further tested for salt tolerance by subculture into G media buffered with CHESS at pH9.0 containing varying amounts of salt (0, 50, 75, 100 mM added NaCl). Cultures that grew in the presence of salt were scaled up for growth in 6-well plates to confirm the phenotype. The number of candidates screened is shown above in Table 4.

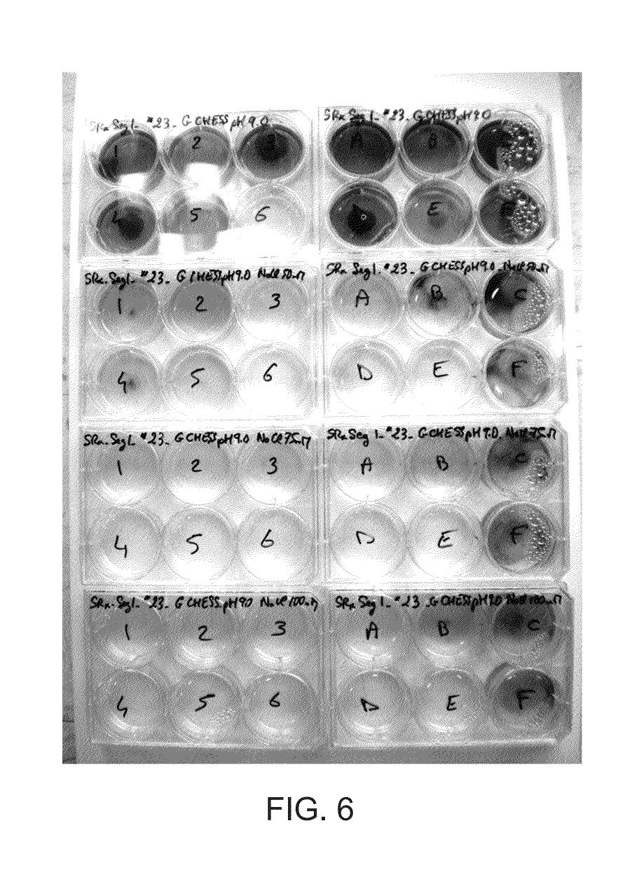

As a further piece of evidence that expression of SR1 leads to the phenotype of salt tolerance, strains containing the SR1 gene were back crossed/mated with a wild type Chlamydomonas strain of the opposite mating type (Chlamydomonas strain CC1691, mt−). Cultures to be mated were grown to early log phase, harvested by centrifugation (4000×g, 10 minutes) and resuspended in media containing no nitrogen source (HSM-NH4) to an equal volume as the original culture. Cultures were then placed at 23° C. under constant illumination of 450 Lux on a rotary shaker set at 100 rpm for 8-16 hours to induce gamete formation. Equal volumes of the mating cultures were mixed and allowed to grow for a further 16-24 hours. Cells from this culture were plated on solid media (HSM-NH4) and placed in light for 5 days. Unmated gametes were killed by chloroform treatment. Plates were placed face down above a chloroform source for 40 seconds. Cells were then cultured in liquid TAP media for 3-5 days before plating on solid media to isolate single colonies. Strains (progeny) that grew were tested for hygromycin resistance. Six strains (progeny) showing hygromycin resistance (thus should contain the SR1 gene and thus should be salt tolerant) and six strains (progeny) showing hygromycin sensitivity (thus should not contain the SR1 gene) were screened for salt tolerance. Strains (progeny) were grown to saturation in liquid media and then diluted in G media buffered with CHESS buffer at pH 9.0 containing 0, 50, 75, 100 mM added NaCl. FIG. 6 shows the results of the mating. The top two rows of cultures were grown in G media buffered with CHESS buffer at pH 9.0 with 0 mM added NaCl: the next two rows of cultures were grown in G media buffered with CHESS buffer at pH 9.0 with 50 mM added NaCl; the next two rows of cultures were grown in G media buffered with CHESS buffer at pH 9.0 with 75 mM added NaCl; and the last two rows of cultures were grown in G media buffered with CHESS buffer at pH 9.0 with 100 mM added NaCl. Dark media indicates growth of the algae and clear media indicates no growth. The left hand three columns show cultures of progeny that were sensitive to hygromycin. The right hand three columns show cultures of progeny that were resistant to hygromycin. All progeny that were sensitive to hygromycin were unable to grow in media containing added NaCl at a concentration above 50 mM. Some progeny that were resistant to hygromycin were also able to grow in media containing added NaCl at a concentration up to at least 100 mM, indicating that the salt resistant phenotype may be the result of the expression of the SR1 gene in the transformed algae. Dark media indicates growth of the algae and clear media indicates no growth.

### Example 7

### Nuclear Transformation of C. reinhardtii with a SR2 Gene that Confers Salt Tolerance

In this example a polynucleotide (SEQ ID NO: 29) encoding SR2 protein (SEQ ID NO: 30) was introduced into C. reinhardtii (CC1690). The plasmid construct (as shown in FIG. 1) contained the gene encoding SR2 that is regulated by the 5′ UTR and promoter sequence for the HSP70A/rbcS2 gene from C. reinhardtii and the 3′ UTR sequence for the rbcS2 gene from C. reinhardtii. A Metal Affinity Tag (MAT), a protease cleavage site (TEV) and FLAG epitope tag were added to the 3′ end of the ORF, using standard techniques. The amino acid sequence of the FLAG-TEV-MAT tag is shown in SEQ ID NO: 28. The same plasmid construct contained the hygromycin resistance gene expressed as a selectable marker regulated by the beta-Tubulin promoter and 5′UTR and rbcs2 3′UTR from C. reinhardtii. The transgene cassette can be flanked by segments of an appropriate nuclear genomic locus of C. reinhardtii for genomic integration of the transgene via homologous recombination if desired. Electroporation, which is a known technique in the art, was used for nuclear transformation. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain CC1690 (mt+). Cells were grown to mid log phase in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Cells were harvested by centrifugation at 4,000×g at 4° C. for 10 min. The supernatant was decanted and cells were resuspended in TAP medium containing 40 mM Sucrose to a final concentration of 3×10̂8 cells/ml for subsequent transformation by electroporation. DNA for use in transformation was first linearized by restriction digest using an enzyme that only has one recognition site within the plasmid construct. DNA for transformation was added to 250 ul cells and placed in an 0.4 cm electroporation cuvette on ice. Conditions for electroporation were 800V, 25 uF, infinite resistance using exponential decay electroporation on a BIORAD gene pulser electroporator. Cells that were successfully transformed with SR2 gene were tolerant to higher concentrations of salt. Transformants were either selected for on media containing Hygromycin (20 ug/ml) or media containing both Hygromycin and salt selection sufficient to prevent growth of the parental strain (greater than 200 mM for TAP media). Strains that grew under these initial selection conditions were grown to saturation in 200 ul liquid cultures in 96-well format in TAP media. These cultures were further tested for salt tolerance by subculture into G media buffered with CHESS at pH9.0 containing varying amounts of salt (0, 50, 75, 100 mM added NaCl). Cultures that grew in the presence of salt were scaled up for growth in 6-well plates to confirm the phenotype. The number of candidates screened is shown above in Table 4.

FIG. 3A shows quantitative analysis of the growth rate of transformed algae and control untransformed algae in the absence of salt (TAP media). Both transformed algae and untransformed control algae all grow in the absence of salt and show a similar growth rate. FIG. 3B shows quantitative analysis of the growth rate of transformed algae and control untransformed algae in the presence of salt (TAP plus 250 mM NaCl). Algae were grown in TAP or TAP plus 250 mM NaCl and cell density measured over time. The graphs show cell density (cells/ml×10⁷) versus time (days). SR2 (#2)(circle) shows a higher growth rate when compared to the control untransformed algae (WT)(square).

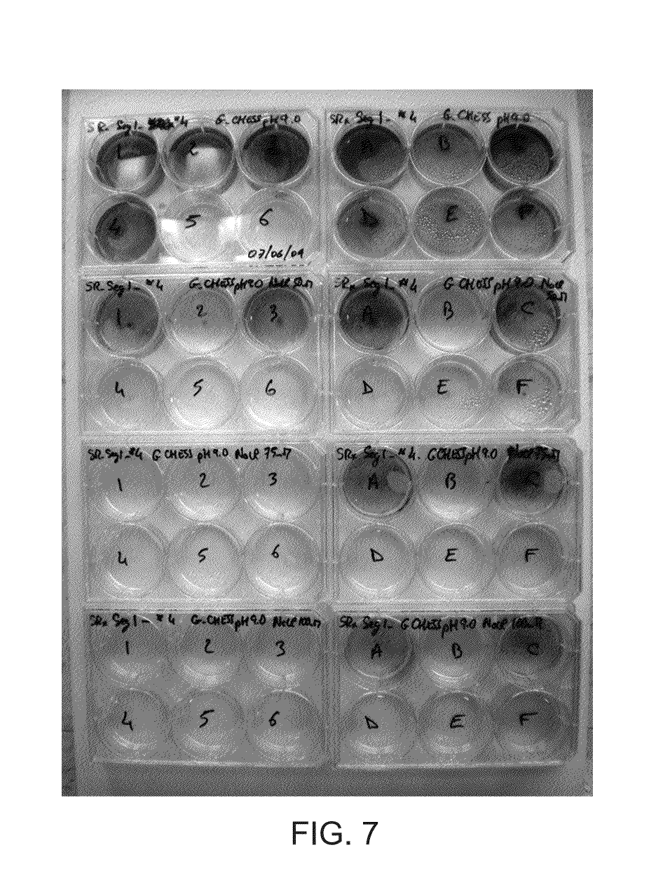

As a further piece of evidence that expression of SR2 leads to the phenotype of salt tolerance, strains containing the SR2 gene were back crossed/mated with a wild type Chlamydomonas strain of the opposite mating type (Chlamydomonas strain CC1691, mt−). Cultures to be mated were grown to early log phase, harvested by centrifugation (4000×g, 10 minutes) and resuspended in media containing no nitrogen source (HSM-NH4) to an equal volume as the original culture. Cultures were then placed at 23° C. under constant illumination of 450 Lux on a rotary shaker set at 100 rpm for 8-16 hours to induce gamete formation. Equal volumes of the mating cultures were mixed and allowed to grow for a further 16-24 hours. Cells from this culture were plated on solid media (HSM-NH4) and placed in light for 5 days. Unmated gametes were killed by chloroform treatment. Plates were placed face down above a chloroform source for 40 seconds. Cells were then cultured in liquid TAP media for 3-5 days before plating on solid media to isolate single colonies. Strains (progeny) that grew were tested for hygromycin resistance. Six strains (progeny) showing hygromycin resistance (thus should contain the SR2 gene and thus should be salt tolerant) and six strains (progeny) showing hygromycin sensitivity (thus should not contain the SR2 gene) were screened for salt tolerance. Strains (progeny) were grown to saturation in liquid media and then diluted in G media buffered with CHESS buffer at pH 9.0 containing 0, 50, 75. 100 mM added NaCl. FIG. 7 shows the results of the mating. The top two rows of cultures were grown in G media buffered with CHESS buffer at pH 9.0 with 0 mM added NaCl; the next two rows of cultures were grown in G media buffered with CHESS buffer at pH 9.0 with 50 mM added NaCl: the next two rows of cultures were grown in G media buffered with CHESS buffer at pH 9.0 with 75 mM added NaCl; and the last two rows of cultures were grown in G media buffered with CHESS buffer at pH 9.0 with 100 mM added NaCl. Dark media indicates growth of the algae and clear media indicates no growth. The left hand three columns show cultures of progeny that were sensitive to hygromycin. The right hand three columns show cultures of progeny that were resistant to hygromycin. All progeny that were sensitive to hygromycin were unable to grow in media containing added NaCl at a concentration above 50 mM. Some progeny that were resistant to hygromycin were also able to grow in media containing added NaCl at a concentration up to at least 100 mM, indicating that the salt resistant phenotype may be the result of the expression of the SR2 gene in the transformed algae. Dark media indicates growth of the algae and clear media indicates no growth.

### Example 8

### Nuclear Transformation of C. reinhardtii with a SR3 Gene that Confers Salt Tolerance

In this example a polynucleotide (SEQ ID NO: 33) encoding SR3 protein (SEQ ID NO: 34) was introduced into C. reinhardtii (CC1690). The plasmid construct (as shown in FIG. 1) contained the gene encoding SR3 that is regulated by the 5′ UTR and promoter sequence for the HSP70A/rbcS2 gene from C. reinhardtii and the 3′ UTR sequence for the rbcS2 gene from C. reinhardtii. A Metal Affinity Tag (MAT), a protease cleavage site (TEV) and FLAG epitope tag were added to the 3′ end of the ORF, using standard techniques. The amino acid sequence of the FLAG-TEV-MAT tag is shown in SEQ ID NO: 28. The same plasmid construct contained the hygromycin resistance gene expressed as a selectable marker regulated by the beta-Tubulin promoter and 5′UTR and rbcs2 3′UTR from C. reinhardtii. The transgene cassette can be flanked by segments of an appropriate nuclear genomic locus of C. reinhardtii for genomic integration of the transgene via homologous recombination if desired. Electroporation, which is a known technique in the art, was used for nuclear transformation. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain CC1690 (mt+). Cells were grown to mid log phase in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Cells were harvested by centrifugation at 4,000×g at 4° C. for 10 min. The supernatant was decanted and cells were resuspended in TAP medium containing 40 mM Sucrose to a final concentration of 3×10̂8 cells/ml for subsequent transformation by electroporation. DNA for use in transformation was first linearized by restriction digest using an enzyme that only has one recognition site within the plasmid construct. DNA for transformation was added to 250 ul cells and placed in an 0.4 cm electroporation cuvette on ice. Conditions for electroporation were 800V, 25 uF, infinite resistance using exponential decay electroporation on a BIORAD gene pulser electroporator. Cells that were successfully transformed with SR3 gene were tolerant to higher concentrations of salt. Transformants were either selected for on media containing Hygromycin (20 ug/ml) or media containing both Hygromycin and salt selection sufficient to prevent growth of the parental strain (greater than 200 mM for TAP media). Strains that grew under these initial selection conditions were grown to saturation in 200 ul liquid cultures in 96-well format in TAP media. These cultures were further tested for salt tolerance by subculture into G media buffered with CHESS at pH9.0 containing varying amounts of salt (0, 50, 75, 100 mM added NaCl). Cultures that grew in the presence of salt were scaled up for growth in 6-well plates to confirm the phenotype. The number of candidates screened is shown above in Table 4.

PCR was used to identify transformed strains. For PCR analysis, 10⁶ algae cells (from agar plate or liquid culture) were suspended in 10 mM EDTA and heated to 95° C. for 10 minutes, then cooled to near 23° C. A PCR cocktail consisting of reaction buffer, MgCl₂. DMSO, Betaine, dNTPs, PCR primer pair(s), DNA polymerase, and water was prepared. Algae lysate in EDTA was added to provide template for reaction. Magnesium concentration was varied to compensate for amount and concentration of algae lysate in EDTA added. Annealing temperature gradients were employed to determine optimal annealing temperature for specific primer pairs.

To identify strains that contained the SR3 gene, a primer pair was used in which one primer annealed to a site within the rbcS2 5′UTR and the other primer annealed within the SR3 coding segment. Desired clones were those that yielded a PCR product of expected size.

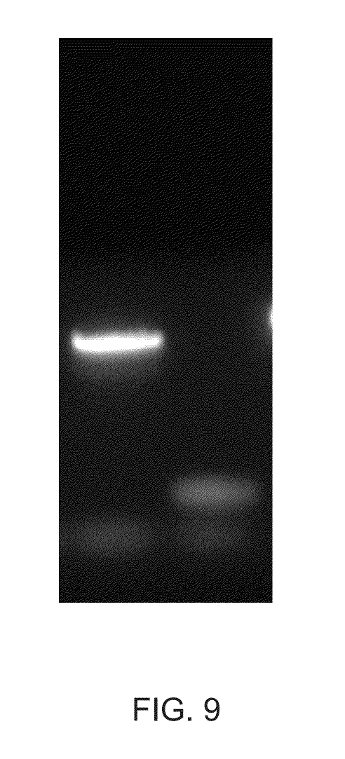

The left column of FIG. 9 shows PCR product using algae transformed with SR3 as template. A band is shown of the expected size for the SR3 gene. The right column of FIG. 9 shows PCR product using untransformed algae as template. No band is seen in this column.

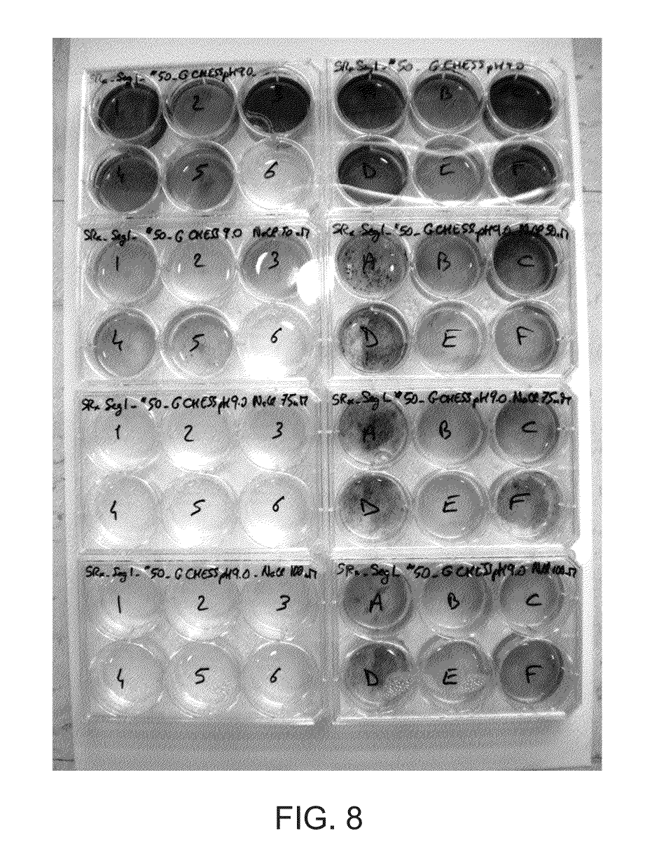

As a further piece of evidence that expression of SR3 leads to the phenotype of salt tolerance, strains containing the SR3 gene were back crossed/mated with a wild type Chlamydomonas strain of the opposite mating type (Chlamydomonas strain CC1691, mt−). Cultures to be mated were grown to early log phase, harvested by centrifugation (4000×g, 10 minutes) and resuspended in media containing no nitrogen source (HSM-NH4) to an equal volume as the original culture. Cultures were then placed at 23° C. under constant illumination of 450 Lux on a rotary shaker set at 100 rpm for 8-16 hours to induce gamete formation. Equal volumes of the mating cultures were mixed and allowed to grow for a further 16-24 hours. Cells from this culture were plated on solid media (HSM-NH4) and placed in light for 5 days. Unmated gametes were killed by chloroform treatment. Plates were placed face down above a chloroform source for 40 seconds. Cells were then cultured in liquid TAP media for 3-5 days before plating on solid media to isolate single colonies. Strains (progeny) that grew were tested for hygromycin resistance. Six strains (progeny) showing hygromycin resistance (thus should contain the SR3 gene and thus should be salt tolerant) and six strains (progeny) showing hygromycin sensitivity (thus should not contain the SR3 gene) were screened for salt tolerance. Strains (progeny) were grown to saturation in liquid media and then diluted in G media buffered with CHESS buffer at pH 9.0 containing 0, 50, 75, 100 mM added NaCl. FIG. 8 shows the results of the mating. The top two rows of cultures were grown in G media buffered with CHESS buffer at pH 9.0 with 0 mM added NaCl; the next two rows of cultures were grown in G media buffered with CHESS buffer at pH 9.0 with 50 mM added NaCl; the next two rows of cultures were grown in G media buffered with CHESS buffer at pH 9.0 with 75 mM added NaCl; and the last two rows of cultures were grown in G media buffered with CHESS buffer at pH 9.0 with 100 mM added NaCl. Dark media indicates growth of the algae and clear media indicates no growth. The left hand three columns show cultures of progeny that were sensitive to hygromycin. The right hand three columns show cultures of progeny that were resistant to hygromycin. All progeny that were sensitive to hygromycin were unable to grow in media containing added NaCl at a concentration above 50 mM. All of the progeny that were resistant to hygromycin were also able to grow in media containing added NaCl at a concentration up to at least 100 mM, indicating that the salt resistant phenotype may be the result of the expression of the SR3 gene in the transformed algae. Dark media indicates growth of the algae and clear media indicates no growth.

### Example 9

### Nuclear Transformation of C. reinhardtii with a SR4 Gene that Confers Salt Tolerance

In this example a polynucleotide (SEQ ID NO: 37) encoding SR4 protein (SEQ ID NO: 38) was introduced into C. reinhardtii (CC1690). The plasmid construct (as shown in FIG. 1) contained the gene encoding SR4 that is regulated by the 5′ UTR and promoter sequence for the HSP70A/rbcS2 gene from C. reinhardtii and the 3′ UTR sequence for the rbcS2 gene from C. reinhardtii. A Metal Affinity Tag (MAT), a protease cleavage site (TEV) and FLAG epitope tag were added to the 3′ end of the ORF, using standard techniques. The amino acid sequence of the FLAG-TEV-MAT tag is shown in SEQ ID NO: 28. The same plasmid construct contained the hygromycin resistance gene expressed as a selectable marker regulated by the beta-Tubulin promoter and 5′UTR and rbcs2 3′UTR from C. reinhardtii. The transgene cassette can be flanked by segments of an appropriate nuclear genomic locus of C. reinhardtii for genomic integration of the transgene via homologous recombination if desired. Electroporation, which is a known technique in the art, was used for nuclear transformation. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297.192-208. 1998.

For these experiments, all transformations were carried out on C. reinhardtii strain CC1690 (mt+). Cells were grown to mid log phase in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Cells were harvested by centrifugation at 4,000×g at 4° C. for 10 min. The supernatant was decanted and cells were resuspended in TAP medium containing 40 mM Sucrose to a final concentration of 3×10̂8 cells/ml for subsequent transformation by electroporation. DNA for use in transformation was first linearized by restriction digest using an enzyme that only has one recognition site within the plasmid construct. DNA for transformation was added to 250 ul cells and placed in an 0.4 cm electroporation cuvette on ice. Conditions for electroporation were 800V, 25 uF, infinite resistance using exponential decay electroporation on a BIORAD gene pulser electroporator. Cells that were successfully transformed with SR4 gene were tolerant to higher concentrations of salt. Transformants were either selected for on media containing Hygromycin (20 ug/ml) or media containing both Hygromycin and salt selection sufficient to prevent growth of the parental strain (greater than 200 mM for TAP media). Strains that grew under these initial selection conditions were grown to saturation in 200 ul liquid cultures in 96-well format in TAP media. These cultures were further tested for salt tolerance by subculture into G media buffered with CHESS at pH9.0 containing varying amounts of salt (0, 50, 75, 100 mM added NaCl). Cultures that grew in the presence of salt were scaled up for growth in 6-well plates to confirm the phenotype. The number of candidates screened is shown above in Table 4.

### Example 10

### Nuclear Transformation of C. reinhardtii with a SR5 Gene that Confers Salt Tolerance

In this example a polynucleotide (SEQ ID NO: 41) encoding SR5 protein (SEQ ID NO: 42) was introduced into C. reinhardtii (CC1690). The plasmid construct (as shown in FIG. 1) contained the gene encoding SR5 that is regulated by the 5′ UTR and promoter sequence for the HSP70A/rbcS2 gene from C. reinhardtii and the 3′ UTR sequence for the rbcS2 gene from C. reinhardtii. A Metal Affinity Tag (MAT), a protease cleavage site (TEV) and FLAG epitope tag were added to the 3′ end of the ORF, using standard techniques. The amino acid sequence of the FLAG-TEV-MAT tag is shown in SEQ ID NO: 28. The same plasmid construct contained the hygromycin resistance gene expressed as a selectable marker regulated by the beta-Tubulin promoter and 5′UTR and rbcs2 3′UTR from C. reinhardtii. The transgene cassette can be flanked by segments of an appropriate nuclear genomic locus of C. reinhardtii for genomic integration of the transgene via homologous recombination if desired. Electroporation, which is a known technique in the art, was used for nuclear transformation. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain CC1690 (mt+). Cells were grown to mid log phase in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Cells were harvested by centrifugation at 4,000×g at 4° C. for 10 min. The supernatant was decanted and cells were resuspended in TAP medium containing 40 mM Sucrose to a final concentration of 3×10̂8 cells/ml for subsequent transformation by electroporation. DNA for use in transformation was first linearized by restriction digest using an enzyme that only has one recognition site within the plasmid construct. DNA for transformation was added to 250 ul cells and placed in an 0.4 cm electroporation cuvette on ice. Conditions for electroporation were 800V, 25 uF, infinite resistance using exponential decay electroporation on a BIORAD gene pulser electroporator. Transformants were either selected for on media containing Hygromycin (20 ug/ml) or media containing both Hygromycin and salt selection sufficient to prevent growth of the parental strain (greater than 200 mM for TAP media). Strains that grew under these initial selection conditions were grown to saturation in 200 ul liquid cultures in 96-well format in TAP media. These cultures were further tested for salt tolerance by subculture into G media buffered with CHESS at pH9.0 containing varying amounts of salt (0, 50, 75, 100 mM added NaCl).

### Example 11

### Nuclear Transformation of C. reinhardtii with a SR6 Gene that Confers Salt Tolerance

In this example a polynucleotide (SEQ ID NO: 45) encoding SR6 protein (SEQ ID NO: 46) was introduced into C. reinhardtii (CC1690). The plasmid construct (as shown in FIG. 1) contained the gene encoding SR6 that is regulated by the 5′ UTR and promoter sequence for the HSP70A/rbcS2 gene from C. reinhardtii and the 3′ UTR sequence for the rbcS2 gene from C. reinhardtii. A Metal Affinity Tag (MAT), a protease cleavage site (TEV) and FLAG epitope tag were added to the 3′ end of the ORF, using standard techniques. The amino acid sequence of the FLAG-TEV-MAT tag is shown in SEQ ID NO: 28. The same plasmid construct contained the hygromycin resistance gene expressed as a selectable marker regulated by the beta-Tubulin promoter and 5′UTR and rbcs2 3′UTR from C. reinhardtii. The transgene cassette can be flanked by segments of an appropriate nuclear genomic locus of C. reinhardtii for genomic integration of the transgene via homologous recombination if desired. Electroporation, which is a known technique in the art, was used for nuclear transformation. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain CC1690 (mt+). Cells were grown to mid log phase in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Cells were harvested by centrifugation at 4,000×g at 4° C. for 10 min. The supernatant was decanted and cells were resuspended in TAP medium containing 40 mM Sucrose to a final concentration of 3×10̂8 cells/ml for subsequent transformation by electroporation. DNA for use in transformation was first linearized by restriction digest using an enzyme that only has one recognition site within the plasmid construct. DNA for transformation was added to 250 ul cells and placed in an 0.4 cm electroporation cuvette on ice. Conditions for electroporation were 800V, 25 uF, infinite resistance using exponential decay electroporation on a BIORAD gene pulser electroporator. Transformants were either selected for on media containing Hygromycin (20 ug/ml) or media containing both Hygromycin and salt selection sufficient to prevent growth of the parental strain (greater than 200 mM for TAP media). Strains that grew under these initial selection conditions were grown to saturation in 200 ul liquid cultures in 96-well format in TAP media. These cultures were further tested for salt tolerance by subculture into G media buffered with CHESS at pH9.0 containing varying amounts of salt (0, 50, 75, 100 mM added NaCl).

### Example 12

### Nuclear Transformation of C. reinhardtii with a SR7 Gene that Confers Salt Tolerance

In this example a polynucleotide (SEQ ID NO: 50) encoding SR7 protein (SEQ ID NO: 51) was introduced into C. reinhardtii (CC1690). The plasmid construct (as shown in FIG. 1) contained the gene encoding SR7 that is regulated by the 5′ UTR and promoter sequence for the HSP70A/rbcS2 gene from C. reinhardtii and the 3′ UTR sequence for the rbcS2 gene from C. reinhardtii. A Metal Affinity Tag (MAT), a protease cleavage site (TEV) and FLAG epitope tag were added to the 3′ end of the ORF, using standard techniques. The amino acid sequence of the FLAG-TEV-MAT tag is shown in SEQ ID NO: 28. The same plasmid construct contained the hygromycin resistance gene expressed as a selectable marker regulated by the beta-Tubulin promoter and 5′UTR and rbcs2 3′UTR from C. reinhardtii. The transgene cassette can be flanked by segments of an appropriate nuclear genomic locus of C. reinhardtii for genomic integration of the transgene via homologous recombination if desired. Electroporation, which is a known technique in the art, was used for nuclear transformation. All DNA manipulations carried out in the construction of this transforming DNA are essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain CC1690 (mt+). Cells were grown to mid log phase in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Cells were harvested by centrifugation at 4,000×g at 4° C. for 10 min. The supernatant was decanted and cells were resuspended in TAP medium containing 40 mM Sucrose to a final concentration of 3×10̂8 cells/ml for subsequent transformation by electroporation. DNA for use in transformation was first linearized by restriction digest using an enzyme that only has one recognition site within the plasmid construct. DNA for transformation was added to 250 ul cells and placed in an 0.4 cm electroporation cuvette on ice. Conditions for electroporation were 800V, 25 uF, infinite resistance using exponential decay electroporation on a BIORAD gene pulser electroporator. Cells that were successfully transformed with SR7 gene were tolerant to higher concentrations of salt. Transformants were either selected for on media containing Hygromycin (20 ug/ml) or media containing both Hygromycin and salt selection sufficient to prevent growth of the parental strain (greater than 200 mM for TAP media). Strains that grew under these initial selection conditions were grown to saturation in 200 ul liquid cultures in 96-well format in TAP media. These cultures were further tested for salt tolerance by subculture into G media buffered with CHESS at pH9.0 containing varying amounts of salt (0, 50, 75, 100 mM added NaCl). Cultures that grew in the presence of salt were scaled up for growth in 6-well plates to confirm the phenotype, and an exemplary screen is shown in FIG. 5. Both the top and bottom panel show four 6-well plates. The upper left plate shows cultures grown in G media buffered with CHESS at pH9.0 containing 0 mM added NaCl. The lower left plate shows cultures grown in G media buffered with CHESS at pH9.0 containing 50 mM added NaCl. The upper right plate shows cultures grown in G media buffered with CHESS at pH9.0 containing 75 mM added NaCl. The lower right plate shows cultures grown in G media buffered with CHESS at pH9.0 containing 100 mM added NaCl. Dark media indicates growth of the algae and clear media indicates no growth. Top panel: the bottom row of each of the four plates, marked “11” contains a culture of algae transformed with SR7, showing growth in media containing up to at least 100 mM added NaCl. Lower panel: the lower row of each of the four plates, containing the marking “21 gr” contain cultures of the untransformed algae, and do not show growth in media containing greater than 50 mM added NaCl.

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A non-vascular photosynthetic organism transformed with an exogenous polynucleotide encoding a protein of SEQ ID NO. 36 or at least 90% sequence identity to SEQ ID NO. 36, wherein said transformed non-vascular photosynthetic organism has a greater salt tolerance than said organism not transformed with said exogenous polynucleotide.

2. The organism of claim 1, wherein said organism is an alga.

3. The organism of claim 2, wherein said organism is an alga of the genus Nannochloropsis, Chlamydomonas, Scenedesmus, or Dunaliella.

4. The organism of claim 1, wherein said organism is a cyanobacterium.

5. The organism of claim 4, wherein said cyanobacterium is of the genus Spirulina.

6. The organism of claim 1, wherein said organism is transformed with a second exogenous polynucleotide.

7. The organism of claim 6, wherein said second exogenous polynucleotide encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a BBC protein or a functional homolog of a BBC protein, or a SCSR protein or a functional homolog of a SCSR protein.

8. The organism of claim 1, wherein said organism is tolerant to a NaCl concentration of at least 25 mM, 50 mM, 75 mM, 100 mM, 150 mM, 200 mM, 250 mM, or 300 mM; or is said NaCl concentration is from 250 mM to 300 mM.

9. A method of increasing the salt tolerance of a non-vascular photosynthetic organism comprising transforming said organism with a polynucleotide encoding a protein of SEQ ID NO. 36 or at least 90% sequence identity to SEQ ID NO. 36, and expressing said protein, wherein said non-vascular photosynthetic organism has an increased salt tolerance as compared to said organism not transformed with said polynucleotide.

10. The method of claim 9, wherein said organism is an alga.

11. The method of claim 10, wherein said organism is an alga of the genus Nannochloropsis, Chlamydomonas, Scenedesmus, or Dunaliella.

12. The method of claim 9, wherein said organism is a cyanobacterium.

13. The method of claim 12, wherein said cyanobacterium is of the genus Spirulina.

14. The method of claim 9, further comprising transforming said organism with a second exogenous polynucleotide sequence.

15. The method of claim 14, wherein said second exogenous polynucleotide sequence encodes for a chaperonin, an antioxidant, a biodegradative enzyme, exo-β-glucanase, endo-β-glucanase, β-glucosidase, endoxylanase, lignase, a flocculating moiety, a botryococcene synthase, a limonene synthase, a 1,8 cineole synthase, a α-pinene synthase, a camphene synthase, a (+)-sabinene synthase, a myrcene synthase, an abietadiene synthase, a taxadiene synthase, a farnesyl pyrophosphate synthase, an amorphadiene synthase, a (E)-α-bisabolene synthase, a diapophytoene synthase, a diapophytoene desaturase, a transporter, a protein that regulates the expression of a transporter, a BBC protein or a functional homolog of a BBC protein, or a SCSR protein or a functional homolog of a SCSR protein.


SEQUENCE LISTING (62 sequences)

SEQ ID NO: 1 - DNA, 2313 bp, Oryza sativa
gagaagagagttttgtagcgagctcgcgcgaatgcgaagccaaccgagagaggtctcgataccaaatcccgatttctcaacctgaatcccccccccacgttcctcgtttcaatctgttcgtctgcgaatcgaattctttgtttttttttctctaattttaccgggaattgtcgaattaggcattcaccaacgagcaagaggggagtggattggttggttaaagctccgcatcttgcggcggaaatctcgctctcttctctgcggtgggtggccggagaagtcgccgccggtgaggcatggggatggaggtggcggcggcgcggctgggggctctgtacacgacctccgactacgcgtcggtggtgtccatcaacctgttcgtcgcgctgctctgcgcctgcatcgtcctcggccacctcctcgaggagaatcgctgggtcaatgagtccatcaccgcgctcatcatcgggctctgcaccggcgtggtgatcttgctgatgaccaaagggaagagctcgcacttattcgtcttcagtgaggatctcttcttcatctacctcctccctccgatcatcttcaatgcaggttttcaggtaaagaaaaagcaattcttccggaatttcatgacgatcacattatttggagccgtcgggacaatgatatcctttttcacaatatctattgctgccattgcaatattcagcagaatgaacattggaacgctggatgtaggagattttcttgcaattggagccatcttttctgcgacagattctgtctgcacattgcaggtcctcaatcaggatgagacaccctttttgtacagtctggtattcggtgaaggtgttgtgaacgatgctacatcaattgtgcttttcaacgcactacagaactttgatcttgtccacatagatgcggctgtcgttctgaaattcttggggaacttcttttatttatttttgtcgagcaccttccttggagtatttgctggattgctcagtgcatacataatcaagaagctatacattggaaggcattctactgaccgtgaggttgcccttatgatgctcatggcttacctttcatatatgctggctgagttgctagatttgagcggcattctcaccgtattcttctgtggtattgtaatgtcacattacacttggcataacgtcacagagagttcaagagttacaacaaagcacgcatttgcaactctgtccttcattgctgagacttttctcttcctgtatgttgggatggatgcattggatattgaaaaatgggagtttgccagtgacagacctggcaaatccattgggataagctcaattttgctaggattggttctgattggaagagctgcttttgtattcccgctgtcgttcttgtcgaacctaacaaagaaggcaccgaatgaaaaaataacctggagacagcaagttgtaatatggtgggctgggctgatgagaggagctgtgtcgattgctcttgcttacaataagtttacaagatctggccatactcagctgcacggcaatgcaataatgatcaccagcaccatcactgtcgttctttttagcactatggtatttgggatgatgacaaagccattgatcaggctgctgctaccggcctcaggccatcctgtcacctctgagccttcatcaccaaagtccctgcattctcctctcctgacaagcatgcaaggttctgacctcgagagtacaaccaacattgtgaggccttccagcctccggatgctcctcaccaagccgacccacactgtccactactactggcgcaagttcgacgacgcgctgatgcgaccgatgtttggcgggcgcgggttcgtgcccttctcccctggatcaccaaccgagcagagccatggaggaagatgaacagtgcaaagaaatgagaatggaatggttgatgaggagaatacatgtaaaatgtgacagcaaaagagagaaggcaagttttgggtttgtagagtttggctgctgctaatgagttgttgatagtgcctatattcttcagaacttcagatggtgcctcaccaaggcctaagagccaggaggaccttctgataatggttcgggatgattggtttgttctgtcaggatgaaccctagtgagtgacacagggtgatgtgctccgacaacctgtaaattttgtagattaacagccccatttgtacctgtctaccatctttagttggcgggtgttctttcctagttgccaccctgcatgtaaaatgaaattctccgccaaaatagatttgtgtgtataataattttgcttggttg

SEQ ID NO: 2 - protein, 535 aa, Oryza sativa
MGMEVAAARLGALYTTSDYASVVSINLFVALLCACIVLGHLLEENRWVNESITALIIGLCTGVVILLMTKGKSSHLFVFSEDLFFIYLLPPIIFNAGFQVKKKQFFRNFMTITLFGAVGTMISFFTISIAAIAIFSRMNIGTLDVGDFLAIGAIFSATDSVCTLQVLNQDETPFLYSLVFGEGVVNDATSIVLFNALQNFDLVHIDAAVVLKFLGNFFYLFLSSTFLGVFAGLLSAYIIKKLYIGRHSTDREVALMMLMAYLSYMLAELLDLSGILTVFFCGIVMSHYTWHNVTESSRVTTKHAFATLSFIAETFLFLYVGMDALDIEKWEFASDRPGKSIGISSILLGLVLIGRAAFVFPLSFLSNLTKKAPNEKITWRQQVVIWWAGLMRGAVSIALAYNKFTRSGHTQLHGNAIMITSTITVVLFSTMVFGMMTKPLIRLLLPASGHPVTSEPSSPKSLHSPLLTSMQGSDLESTTNIVRPSSLRMLLTKPTHTVHYYWRKFDDALMRPMFGGRGFVPFSPGSPTEQSHGGR

SEQ ID NO: 3 - DNA, 1614 bp, Arabidopsis thaliana
atgttggattctctagtgtcgaaactgccttcgttatcgacatctgatcacgcttctgtggttgcgttgaatctctttgttgcacttctttgtgcttgtattgttcttggtcatcttttggaagagaatagatggatgaacgaatccatcaccgccttgttgattgggctaggcactggtgttaccattttgttgattagtaaaggaaaaagctcgcatcttctcgtctttagtgaagatcttttcttcatatatcttttgccacccattatattcaatgcagggtttcaagtaaaaaagaagcagtttttccgcaatttcgtgactattatgctttttggtgctgttgggactattatttcttgcacaatcatatctctaggtgtaacacagttctttaagaagttggacattggaacctttgacttgggtgattatcttgctattggtgccatatttgctgcaacagattcagtatgtacactgcaggttctgaatcaagacgagacacctttgctttacagtcttgtattcggagagggtgttgtgaatgatgcaacgtcagttgtggtcttcaacgcgattcagagctttgatctcactcacctaaaccacgaagctgcttttcatcttcttggaaacttcttgtatttgtttctcctaagtaccttgcttggtgctgcaaccggtctgataagtgcgtatgttatcaagaagctatactttggaaggcactcaactgaccgagaggttgcccttatgatgcttatggcgtatctttcttatatgcttgctgagcttttcgacttgagcggtatcctcactgtgtttttctgtggtattgtgatgtcccattacacatggcacaatgtaacggagagctcaagaataacaacaaagcatacctttgcaactttgtcatttcttgcggagacatttattttcttgtatgttggaatggatgccttggacattgacaagtggagatccgtgagtgacacaccgggaacatcgatcgcagtgagctcaatcctaatgggtctggtcatggttggaagagcagcgttcgtctttccgttatcgtttctatctaacttagccaagaagaatcaaagcgagaaaatcaactttaacatgcaggttgtgatttggtggtctggtctcatgagaggtgctgtatctatggctcttgcatacaacaagtttacaagggccgggcacacagatgtacgcgggaatgcaatcatgatcacgagtacgataactgtctgtctttttagcacagtggtgtttggtatgctgaccaaaccactcataagctacctattaccgcaccagaacgccaccacgagcatgttatctgatgacaacaccccaaaatccatacatatccctttgttggaccaagactcgttcattgagccttcagggaaccacaatgtgcctcggcctgacagtatacgtggcttcttgacacggcccactcgaaccgtgcattactactggagacaatttgatgactccttcatgcgacccgtctttggaggtcgtggctttgtaccctttgttccaggttctccaactgagagaaaccctcctgatcttagtaaggct

SEQ ID NO: 4 - DNA, 1341 bp, Arabidopsis thaliana
atgacaaagaaaatgagaagagtgggcaagtacgaggttggtcgcacaataggtgaaggaacctttgctaaggttaagtttgcgaggaacacagacactggtgataatgtagccatcaaaattatggctaagagtacaatacttaagaacagaatggttgatcagataaaaagagagatatctataatgaagattgttcgtcacccgaacatagtgaggttgtatgaggtgttggcgagtccttcgaaaatatatatagttttggagtttgtgacaggaggagagctctttgatagaattgttcataaagggaggcttgaagaaagtgagtctcggaaatactttcaacagcttgtagatgctgttgctcattgtcactgcaagggtgtttaccaccgtgacctaaagccagaaaatcttttactcgatacaaatggaaatctgaaggtttcggatttcggactcagtgcattgcctcaggaaggagtagaacttctgcgtgacacatgtggaactccgaactatgtagctccagaggtacttagtggacagggttacgatggttcagcagctgatatttggtcttgcggggttattcttttcgttatattggctggatatttacctttttccgagacggatcttccagggttgtacagaaaaataaatgcagcagagttttcttgtccaccgtggttttccgcagaagtgaagtttttaatacataggatacttgaccccaatcccaaaacacgtattcaaattcaaggaatcaagaaagatccttggttcagattaaattatgtgcctatacgagcaagggaagaagaagaagtgaatttggatgatattcgtgcagtttttgatggaattgagggcagttatgtagcggagaatgtagagagaaatgatgaagggcccctgatgatgaatgcctttgagatgattaccttatcacaaggcttaaatttatctgcactatttgacaggcgacaggattttgttaaaaggcaaacccgttttgtttctcgaagggaacctagtgagataattgctaacattgaggctgtagcgaactcaatgggttttaagtctcatacacgaaacttcaagacaaggctcgagggattatcttcgatcaaggccggacagttagctgttgtgatagagatttacgaggtggcaccatcgcttttcatggtagacgtaagaaaggctgctggtgaaactcttgaatatcacaagttctacaagaagctatgttcgaaactggaaaacataatatggagggcaacagaaggaataccaaagtcagagattctcagaacaatcacgttttga

SEQ ID NO: 5 - protein, 446 aa, Arabidopsis thaliana
MTKKMRRVGKYEVGRTIGEGTFAKVKFARNTDTGDNVAIKIMAKSTILKNRMVDQIKREISIMKIVRHPNIVRLYEVLASPSKIYIVLEFVTGGELFDRIVHKGRLEESESRKYFQQLVDAVAHCHCKGVYHRDLKPENLLLDTNGNLKVSDFGLSALPQEGVELLRDTCGTPNYVAPEVLSGQGYDGSAADIWSCGVILFVILAGYLPFSETDLPGLYRKINAAEFSCPPWFSAEVKFLIHRILDPNPKTRIQIQGIKKDPWFRLNYVPIRAREEEEVNLDDIRAVFDGIEGSYVAENVERNDEGPLMMNAFEMITLSQGLNLSALFDRRQDFVKRQTRFVSRREPSEIIANIEAVANSMGFKSHTRNFKTRLEGLSSIKAGQLAVVIEIYEVAPSLFMVDVRKAAGETLEYHKFYKKLCSKLENIIWRATEGIPKSEILRTITF

SEQ ID NO: 6 - DNA, 927 bp, Arabidopsis thaliana
atgacaaagaaaatgagaagagtgggcaagtacgaggttggtcgcacaataggtgaaggaacctttgctaaggttaagtttgcgaggaacacagacactggtgataatgtagccatcaaaattatggctaagagtacaatacttaagaacagaatggttgatcagataaaaagagagatatctataatgaagattgttcgtcacccgaacatagtgaggttgtatgaggtgttggcgagtccttcgaaaatatatatagttttggagtttgtgacaggaggagagctctttgatagaattgttcataaagggaggcttgaagaaagtgagtctcggaaatactttcaacagcttgtagatgctgttgctcattgtcactgcaagggtgtttaccaccgtgacctaaagccagaaaatcttttactcgatacaaatggaaatctgaaggtttcggatttcggactcagtgcattgcctcaggaaggagtagaacttctgcgtgacacatgtggaactccgaactatgtagctccagaggtacttagtggacagggttacgatggttcagcagctgatatttggtcttgcggggttattcttttcgttatattggctggatatttacctttttccgagacggatcttccagggttgtacagaaaaataaatgcagcagagttttcttgtccaccgtggttttccgcagaagtgaagtttttaatacataggatacttgaccccaatcccaaaacacgtattcaaattcaaggaatcaagaaagatccttggttcagattaaattatgtgcctatacgagcaagggaagaagaagaagtgaatttggatgatattcgtgcagtttttgatggaattgagggcagttatgtagcggagaatgtagagagaaatgatgaagggcccctgtga

SEQ ID NO: 7 - protein, 308 aa, Arabidopsis thaliana
MTKKMRRVGKYEVGRTIGEGTFAKVKFARNTDTGDNVAIKIMAKSTILKNRMVDQIKREISIMKIVRHPNIVRLYEVLASPSKIYIVLEFVTGGELFDRIVHKGRLEESESRKYFQQLVDAVAHCHCKGVYHRDLKPENLLLDTNGNLKVSDFGLSALPQEGVELLRDTCGTPNYVAPEVLSGQGYDGSAADIWSCGVILFVILAGYLPFSETDLPGLYRKINAAEFSCPPWFSAEVKFLIHRILDPNPKTRIQIQGIKKDPWFRLNYVPIRAREEEEVNLDDIRAVFDGIEGSYVAENVERNDEGPL

SEQ ID NO: 8 - DNA, 990 bp, Arabidopsis thaliana
atgacaaagaaaatgagaagagtgggcaagtacgaggttggtcgcacaataggtgaaggaacctttgctaaggttaagtttgcgaggaacacagacactggtgataatgtagccatcaaaattatggctaagagtacaatacttaagaacagaatggttgatcagataaaaagagagatatctataatgaagattgttcgtcacccgaacatagtgaggttgtatgaggtgttggagagtccttcgaaaatatatatagttttggagtttgtgacaggaggagagctctttgatagaattgttcataaagggaggcttgaagaaagtgagtctcggaaatactttcaacagcttgtagatgctgttgctcattgtcactgcaagggtgtttaccaccgtgacctaaagccagaaaatcttttactcgatacaaatggaaatctgaaggtttcggatttcggactcagtgcattgcctcaggaaggagtagaacttctgcgtgacacatgtggaactccgaactatgtagctccagaggtacttagtggacagggttacgatggttcagcagctgatatttggtcttgcggggttattcttttcgttatattggctggatatttacctttttccgagacggatcttccagggttgtacagaaaaataaatgcagcagagttttcttgtccaccgtggttttccgcagaagtgaagtttttaatacataggatacttgaccccaatcccaaaacacgtattcaaattcaaggaatcaagaaagatccttggttcagattaaattatgtgcctatacgagcaagggaagaagaagaagtgaatttggatgatattcgtgcagtttttgatggaattgagggcagttatgtagaggagaatgtagagagaaatgatgaagggcccctgatgatgaatgcctttgagatgattaccttatcacaaggcttaaatttatctgcactatttgactga

SEQ ID NO: 9 - protein, 329 aa, Arabidopsis thaliana
MTKKMRRVGKYEVGRTIGEGTFAKVKFARNTDTGDNVAIKIMAKSTILKNRMVDQIKREISIMKIVRHPNIVRLYEVLASPSKIYIVLEFVTGGELFDRIVHKGRLEESESRKYFQQLVDAVAHCHCKGVYHRDLKPENLLLDTNGNLKVSDFGLSALPQEGVELLRDTCGTPNYVAPEVLSGQGYDGSAADIWSCGVILFVILAGYLPFSETDLPGLYRKINAAEFSCPPWFSAEVKFLIHRILDPNPKTRIQIQGIKKDPWFRLNYVPIRAREEEEVNLDDIRAVFDGIEGSYVAENVERNDEGPLMMNAFEMITLSQGLNLSALFD

SEQ ID NO: 10 - DNA, 1278 bp, Arabidopsis thaliana
atgacaaagaaaatgagaagagtgggcaagtacgaggttggtcgcacaataggtgaaggaacctttgctaaggttaagtttgcgaggaacacagacactggtgataatgtagccatcaaaattatggctaagagtacaatacttaagaacagaatggttgatcagataaaaagagagatatctataatgaagattgttcgtcacccgaacatagtgaggttgtatgaggtgttggcgagtccttcgaaaatatatatagttttggagtttgtgacaggaggagagctctttgatagaattgttcataaagggaggcttgaagaaagtgagtctcggaaatactttcaacagcttgtagatgctgttgctcattgtcactgcaagggtgtttaccaccgtgacctaaagccagaaaatcttttactcgatacaaatggaaatctgaaggtttcggatttcggactcagtgcattgcctcaggaaggagtagaacttctgcgtgacacatgtggaactccgaactatgtagctccagaggtacttagtggacagggttacgatggttcagcagctgatatttggtcttgcggggttattcttttcgttatattggctggatatttacctttttccgagacggatcttccagggttgtacagaaaaataaatgcagcagagttttcttgtccaccgtggttttccgcagaagtgaagtttttaatacataggatacttgaccccaatcccaaaacacgtattcaaattcaaggaatcaagaaagatccttggttcagattaaattatgtgcctatacgagcaagggaagaagaagaagtgaatttggatgatattcgtgcagtttttgatggaattgagggcagttatgtagcggagaatgtagagagaaatgatgaagggcccctgaggcgacaggattttgttaaaaggcaaacccgttttgtttctcgaagggaacctagtgagataattgctaacattgaggctgtagcgaactcaatgggttttaagtctcatacacgaaacttcaagacaaggctcgagggattatcttcgatcaaggccggacagttagctgttgtgatagagatttacgaggtggcaccatcgcttttcatggtagacgtaagaaaggctgctggtgaaactcttgaatatcacaagttctacaagaagctatgttcgaaactggaaaacataatatggagggcaacagaaggaataccaaagtcagagattctcagaacaatcacgttttga

SEQ ID NO: 11 - protein, 425 aa, Arabidopsis thaliana
MTKKMRRVGKYEVGRTIGEGTFAKVKFARNTDTGDNVAIKIMAKSTILKNRMVDQIKREISIMKIVRHPNIVRLYEVLASPSKIYIVLEFVTGGELFDRIVHKGRLEESESRKYFQQLVDAVAHCHCKGVYHRDLKPENLLLDTNGNLKVSDFGLSALPQEGVELLRDTCGTPNYVAPEVLSGQGYDGSAADIWSCGVILFVILAGYLPFSETDLPGLYRKINAAEFSCPPWFSAEVKFLIHRILDPNPKTRIQIQGIKKDPWFRLNYVPIRAREEEEVNLDDIRAVFDGIEGSYVAENVERNDEGPLRRQDFVKRQTRFVSRREPSEIIANIEAVANSMGFKSHTRNFKTRLEGLSSIKAGQLAVVIEIYEVAPSLFMVDVRKAAGETLEYHKFYKKLCSKLENIIWRATEGIPKSEILRTITF

SEQ ID NO: 12 - DNA, 669 bp, Arabidopsis thaliana
atgggctgctctgtatcgaagaagaagaagaagaatgcaatgcgaccaccgggatatgaggatcccgagcttcttgcatccgtcacgccattcacggtagaagaagtggaggctttgtatgaactgttcaagaagctaagcagctcaattatcgatgatggtcttattcataaggaagaatttcagctggctttattcagaaataggaaccggaggaatctcttcgctgatcggatatttgatgtatttgatgtgaagcgaaatggagtgatcgagtttggtgaatttgtccggtccttaggtgtcttccatccaagcgcgccggtccatgaaaaagtcaaatttgctttcaagttgtacgatttacgacaaactggattcatcgagcgagaagaattgaaagagatggtagtagcgcttcttcacgaatccgaactagttctttccgaagatatgattgaagtaatggtggataaggctttcgtgcaagcagaccgcaaaaacgacggaaaaatcgatatagatgaatggaaagactttgtatccttgaatccatcgctcatcaagaacatgactttgccatatctaaaggacataaataggacgtttccaagtttcgtttcatcttgtgaagaggaagaaatggaattgcaaaacgtatcttcctaa

SEQ ID NO: 13 - protein, 222 aa, Arabidopsis thaliana
MGCSVSKKKKKNAMRPPGYEDPELLASVTPFTVEEVEALYELFKKLSSSIIDDGLIHKEEFQLALFRNRNRRNLFADRIFDVFDVKRNGVIEFGEFVRSLGVFHPSAPVHEKVKFAFKLYDLRQTGFIEREELKEMVVALLHESELVLSEDMIEVMVDKAFVQADRKNDGKIDIDEWKDFVSLNPSLIKNMTLPYLKDINRTFPSFVSSCEEEEMELQNVSS

SEQ ID NO: 14 - DNA, 489 bp, Chlamydomonas reinhardtii
atggcgaaccccgagttttacggcctgtcgacgaccacgctttcgggacagccctttcctttcaaggaccttgagggcaaggcagtcctaatcgtgaacgtggctagcaagtgcggctttacgccacaatacaagggtctggaggagctgtaccaacagtacaaggaccgggggctcgtcatccttggcttcccctgcaaccagtttggaggccaggagcctggggacgctagcgccatcggggagttctgccagcgcaatttcggcgttacgttccccattatggagaagtcggacgtgaacggcaacgacgccaaccccgtgttcaagtacctgaagagccaaaagaagcagttcatgatggagatgattaaatggaactttgagaagttcctggtggacaagagcggccaggtggtggcgcgcttcagcagcatggctacccccgccagcctggcgccggagattgagaaggtgctgaacgcgtaa

SEQ ID NO: 15 - DNA, 483 bp, Artificial Sequence
  Synthetic DNA sequence
gcgaaccccgagttttacggcctgtccacgaccacgctttcgggacagccctttcctttcaaggaccttgagggcaaggcagtcctaatcgtgaacgtggcaagcaagtgcggctttacgccacaatacaagggtctggaggagctgtaccaacagtacaaggaccgggggctcgtcatccttggcttcccctgcaaccagtttggaggccaggagcctggggacgcaagcgccatcggggagttctgccagcgcaatttcggcgttacgttccccattatggagaagtcggacgtgaacggcaacgacgccaaccccgtgttcaagtacctgaagagccaaaagaagcagttcatgatggagatgattaaatggaactttgagaagttcctggtggacaagagcggccaggtggtggcgcgcttcagcagcatggctacccccgccagcctggcgccggagattgagaaggtgctgaacgcg

SEQ ID NO: 16 - protein, 171 aa, Saccharomyces cerevisiae
MGHHHHHHMSEFYKLAPVDKKGQPFPFDQLKGKVVLIVNVASKCGFTPQYKELEALYKRYKDEGFTIIGFPCNQFGHQEPGSDEEIAQFCQLNYGVTFPIMKKIDVNGGNEDPVYKFLKSQKSGMLGLRGIKWNFEKFLVDKKGKVYERYSSLTKPSSLSETIEELLKEVE

SEQ ID NO: 17 - DNA, 792 bp, Chlamydomonas
atgtccgccgacgccgagaagcagtcgctgctggcgacgggcgtgcccgcgcacgctgcgggcgatgcgccgaaggtcgcgccgcgcgagtggcgccaccgctggtacgccatcctcggcgactgctccgcgcccgacgtcgtgtcatgcctgctggcgtggaagcttccgtttgtggcgtgggcgtggaaccagaaccgcgcactggggatgtcgttctggcgcgagctgctgcgcttcgcggtcatcgtcgttggctttgtggtggccacgcacgtcgcgtactgcggcgtcatgatggccatgtgcccggagatccatgaccgcgatggtgccagcgtcgacggcggcccaggcatgatgcgcaagctgctgcacatgcaccagcaccacagtcaccaccacgacgacgactccaccgacgactccaccgacagccacgaccacggcatgtggggcgaagacggcccgcacggcatcccgagggagtgcgtcgcgcgcgtcgcgccagcctacgtggccatcaccggcgtcttcctcgcgctcgcggtctacatgaccctgttcttcgcacgccgccgcacagcgctgcgcgagcgctacggcatcgccggcaccgcgcgcgaggactgcctgctgtacgcgttctgcacgccgtgcgcgctcgcacaggagacgcgcacgctcatccacgagcaggtgcacgacggcatctggtacggcgcgctgccgggcgtcgcgccgccggccgcgacggtcgccgcccccgcgccgcagaagatggcggtgtga

SEQ ID NO: 18 - DNA, 573 bp, Artificial Sequence
  Codon optimized sequence
atggcgaaccccgagttttacggcctgtccacgaccacgctttcgggacagccctttcctttcaaggaccttgagggcaaggcagtcctaatcgtgaacgtggcaagcaagtgcggctttacgccacaatacaagggtctggaggagctgtaccaacagtacaaggaccgggggctcgtcatccttggcttcccctgcaaccagtttggaggccaggagcctggggacgcaagcgccatcggggagttctgccagcgcaatttcggcgttacgttccccattatggagaagtcggacgtgaacggcaacgacgccaaccccgtgttcaagtacctgaagagccaaaagaagcagttcatgatggagatgattaaatggaactttgagaagttcctggtggacaagagcggccaggtggtggcgcgcttcagcagcatggctacccccgccagcctggcgccggagattgagaaggtgctgaacgcgaccggtgactacaaggacgacgacgacaagagcggcgagaacctgtactttcagggccacaaccaccgccacaagcacaccggttaa

SEQ ID NO: 19 - DNA, 560 bp, Artificial Sequence
  Codon optimized sequence
atgtcccatttctacgacctggctcctaaggataaggatggtaacccgtttcccttcagcaacctgaagggcaaggtggtgctcgtggtgaacaccgcctccaagtgcggctttacgcctcagtacaaggggctggaggccctgtaccagaagtataaggaccgcggcttcatcatcctgggcttcccgtgcaaccagttcggcaaccaggagccgggctccgacgaggagatcgcccagttctgccaaaagaactacggcgtgaccttcccggtgctggccaagatcaacgtgaacggcgacaacgtggaccctgtctaccagttcctgaagagccagaagaagcagctgggcctggagcgcatcaagtggaacttcgagaagttcctggtgaaccggcaagggcaggtgatcgagcgctacagctccatctcgaagccggagcatctggagaacgacatcgagtcggtcctgaccggtgactacaaggacgacgacgacaaagcggcgagaacctgtactttcagggccacaaccaccgccacaagcacaccggttaa

SEQ ID NO: 20 - DNA, 1752 bp, Artificial Sequence
  Codon optimized sequence
atgtggagccagctctccagcctgctgtcgggtaagatggacgcgctgaccacctcggaccacgcctccgtggtgtcgatgaacctgttcgtggcgctgctgtgcggttgcattgtgatcgggcacctcctggaggagaaccgctggatgaacgagagcattaccgccctgctgatcggtctcgcgaccggcgtggtgatcctcctgatctcgggcggcaagagcagccacctgctggtgttcagcgaggatctgttcttcatttacctgctgccgcccatcatcttcaacgcgggctttcaggtgaagaagaagcagttctttcgcaacttcatcaccatcgtgctgttcggcgccgtgggcacgctggtgagcttcaccatcatttccctcggcgcgctgagcatcttcaagaagctggacatcggcaccctggagctggccgactacctggcgatcggcgctatcttcgcggccacggactcggtgtgcaccctgcaggtgctcaaccaggacgagactcctctcctgtattccctcgtgttcggcgagggggtggtgaacgacgccacgtccgtggtgctctttaacgccatccaaagcttcgacctgacccgcatcgaccaccgcatcgccctgcagttcatgggcaacttcctgtacctgttcatcgcgtccacgattctgggcgcgttcacgggcctgctgagcgcctacatcattaagaagctgtacttcggccgccactcgaccgaccgcgaggtcgcgctgatgatgctgatggcttacctgagctacatgctggccgagctgttctacctgtcgggcattctcacggtcttcttctgcgggatcgtcatgagccactacacctggcacaacgtgacggagtcgtcccgcgtgaccaccaagcacgctttcgctacgctgagcttcgtcgctgaggtgttcctgtttctgtacgtcggtatggacgccctcgacatcgagaagtggcggtttgtgagcgatagccccggcatctcggtcgccgtgagctcgatcctgctgggcctggtgatggtcgggcgcgcggccttcgtgttccccctgagctggctgatgaacttcgccaagaagagccagtccgagaaggtgacgttcaaccagcagatcgtgatctggtgggcgggcctgatgcgcggggcggtgtccatggcgctggcctacaaccaattcactcgctcgggccacacccagctgcgcggcaacgccatcatgattacttcgacgattagcgtggtgctgttctcgactatggtgttcggcctgctcaccaagcccctgatcatgttcctcctgccgcagcccaagcacttcacctcctgctccaccgtgtccgacgtgggcagcccgaagtcctattcgctccctctgctggagggcaaccaggattacgaggtggacgtcggcaacggcaaccatgaggacacgaccgagccccgcactatcgtgcgcccgtcgagcctgcgcatgctgctgaacgctcccacgcataccgtccaccactactggcggaagttcgacgactccttcatgcgccccgtctttggcggccggggcttcgtccctttcgtcccgggttcccccacggagcagtccaccaacaacctggtggaccggaccaccggtgactacaaggacgacgacgacaagagcggcgagaacctgtactttcagggccacaaccaccgccacaagcacaccggttaa

SEQ ID NO: 21 - DNA, 1701 bp, Artificial Sequence
  Codon optimized sequence
atgctggattccctggtgagcaagctgccttcgctgtccacctcggaccacgccagcgtggtggccctgaacctcttcgtcgccctgctgtgcgcgtgcatcgtcctgggccacctgctggaggagaaccgctggatgaacgagagcatcacggcgctgctgatcggcctcgggacgggcgtcacgatcctgctgatctccaagggtaagagctcgcacctcctggtcttctcggaggacctcttcttcatctatctgctgccgccgatcatcttcaacgcgggcttccaggtgaagaagaagcaattcttccgcaacttcgtgacgattatgctgttcggcgcggtggggaccatcatctcctgcacgatcatttcgctgggcgtgacgcagttcttcaagaagctcgacatcggcaccttcgacctgggcgactacctggcgatcggtgccatcttcgccgcgaccgactccgtgtgcaccctgcaggtgctgaaccaggacgagacgcccctgctgtactcgctggtgtttggcgagggcgtggtgaacgatgccacctcggtggtggtgttcaacgctatccagtcgttcgacctgactcacctgaaccacgaggccgcgttccatctgctcgggaactttctgtacctgttcctgctcagcaccctgctgggcgcggctacgggcctgatcagcgcgtacgtgattaagaagctgtacttcggccgccacagcacggaccgggaggtggccctcatgatgctgatggcttacctgagctacatgctggccgagctgttcgacctcagcggcatcctgacggtgtttttctgcggcattgtgatgtcgcactacacctggcacaacgtcaccgagtcgtcgcggatcaccactaagcacacctttgccaccctgagctttctggccgagaccttcatcttcctgtacgtgggcatggacgcgctggacatcgataagtggcgcagcgtcagcgacacccccggcaccagcatcgccgtgagctcgatcctcatgggcctggtcatggtgggccgcgccgcgttcgtcttcccgctgagcttcctgtcgaacctggcgaagaagaaccagtcggagaagatcaacttcaacatgcaggtggtgatttggtggagcggcctcatgcggggcgccgtgtccatggctctcgcgtacaacaagttcacccgcgccggccacaccgacgtgcgcggcaacgcgattatgattaccagcacgatcaccgtgtgcctgttctcgaccgtggtctttgggatgctgactaagcctctgatctcctacctgctgccccatcagaacgccacgacctccatgctgtccgacgacaacaccccgaagtccatccacatccccctcctggaccaggattccttcatcgagccctccggcaaccacaacgtgccccgccccgacagcattcggggcttcctgactcgcccgacccggaccgtgcactactactggcgccaattcgacgacagctttatgcgcccggtgttcggtggtcgcgggttcgtgcccttcgtccccggctccccgacggagcgcaacccgcctgacctgtccaaggctaccggtgactacaaggacgacgacgacaagagcggcgagaacctgtactttcagggccacaaccaccgccacaagcacaccggttaa

SEQ ID NO: 22 - DNA, 3525 bp, Artificial Sequence
  Codon optimized sequence
atgaccaccgtgattgatgctaccatggcttatcgctttctggaggaggccactgacagctcgtcgtccagcagctcgtccaagctggagtcctcccccgtggacgctgtcctgttcgtgggcatgtcgctcgtgctcgggatcgcgtcccggcacctgctgcgcgggactcgcgtgccgtacaccgtggccctcctggtgatcggcattgccctgggctccctggagtacggcgccaagcacaacctgggcaagatcggccacggcatccggatctggaacgagattgacccggagctgctgctcgccgtgttcctgccggccctgctgtttgagtcgagcttcagcatggaggtgcaccagatcaagcgctgcctgggccagatggtcctgctggccgtgccgggtgtgctgatctcgaccgcttgcctgggctcgctcgtcaaggtgaccttcccctacgagtgggactggaagacgtcgctgctgctgggcggcctgctgagcgccaccgacccggtggccgtggtggcgctgctgaaggagctcggtgcgagcaagaagctgagcaccatcatcgagggcgagagcctgatgaacgacggcactgcgatcgtggtgttccagctgttcctgaagatggcgatggggcagaactccgactggtcgagcatcatcaagttcctgctgaaggtggctctcggcgccgtgggcatcggtctcgccttcggcatcgcctcggtcatctggctgaagttcatcttcaacgacacggtgatcgagattacgctgacgattgcggtgtcgtactttgcgtactacaccgcgcaggagtgggccggtgcgtcgggcgtgctgaccgtcatgacgctgggcatgttctacgcggcgttcgcccggacggcgttcaagggcgacagccagaagagcctgcaccacttctgggagatggtggcctacatcgcgaacaccctcatcttcatcctgagcggcgtggtgattgcggagggcatcctcgactccgacaagatcgcctaccagggcaactcgtggcgcttcctgttcctcctgtacgtgtacattcagctgagccgcgtggtggtcgtgggcgtgctctacccgctgctgtgccgctttggctacggcctggactggaaggagtccatcatcctggtgtggagcggcctgcgcggcgccgtcgctctggccctgtcgctgtccgtgaagcagagctcgggtaactcgcacatctccaaggagaccggcaccctgttcctgttcttcaccggcggtattgtcttcctgacgctcatcgtgaacggcagcaccacccagttcgtgctgcgcctgctgcgcatggacatcctgcctgcccccaagaagcgcatcctggagtataccaagtacgagatgctgaacaaggcgctgcgcgcttttcaggacctgggtgacgatgaggagctgggccccgccgattggcccacggtggagagctacatctcgtccctgaaggggagcgagggggagctggtccaccacccccacaacggctcgaagatcggcagcctggaccccaagtcgctcaaggacatccggatgcgctttctgaacggcgtccaggcgacctactgggagatgctggacgaggggcgcatcagcgaggtgaccgccaacatcctcatgcagtccgtggacgaggcgctggaccaggtcagcaccaccctgtgcgattggcgcggtctgaagccccatgtcaacttccctaactactacaacttcctgcactcgaaggtggtgccgcggaagctggtcacctacttcgccgtggagcgcctggagtccgcgtgctacatttcggcggctttcctgcgcgctcacaccatcgcccggcagcagctgtacgacttcctcggcgagtccaacatcggcagcattgtcatcaacgagtcggagaaggagggcgaggaggcgaagaagtttctggagaaggtccggagctccttcccgcaggtgctgcgcgtggtgaagactaagcaggtgacgtacagcgtgctgaaccatctgctgggctacatcgagaacctggagaaggtgggcctcctggaggagaaggagatcgcgcacctgcacgacgccgtgcagaccgggctgaagaagctgctgcgcaacccgcctatcgtgaagctgcccaagctgtcggacatgatcacctcccaccccctgtcggtcgccctgccgccggcgttctgcgagcccctgaagcatagcaagaaggagcccatgaagctgcgcggcgtgacgctgtacaaggaggggagcaagcctacgggcgtctggctgatcttcgacggcatcgtgaagtggaagagcaagatcctgtcgaacaaccactccctccacccgacgttctcgcacgggtccacgctggggctctacgaggtcctgacgggcaagccgtacctgtgcgacctcatcacggactccatggtgctgtgcttcttcattgattccgagaagatcctcagcctgcaatcggacagcactatcgacgatttcctgtggcaggagtcggccctggtgctgctcaagctgctccggcctcagatcttcgagtccgtcgcgatgcaggagctgcgcgcgctcgtgtccacggagagctccaagctgaccacgtacgtgaccggcgagtcgatcgagatcgactgcaactcgatcggcctcctgctggagggcttcgtcaagcccgtcggcatcaaggaggagctgattagctcccccgccgccctgtccccctcgaacggcaaccagtcctttcacaacagctccgaggccagcggcatcatgcgcgtgtcgttcagccagcaggcgacccaatacatcgtggagacgcgggcccgggctatcattttcaacattggcgccttcggtgccgaccgcaccctgcaccgccgcccctccagcctgaccccgccccgctcgtccagctccgaccagctgcagcggagcttccgcaaggagcaccgcgggctgatgtcgtggcccgagaacatctacgcgaagcagcagcaggagattaacaagaccacgctgtccctgtcggagcgcgccatgcagctgagcattttcggctcgatggtgaacgtgtatcgccggagcgtgagcttcggcggcatctacaacaacaagctgcaggacaacctgctgtataagaagctgcctctgaaccccgcgcagggcctcgtgagcgccaagagcgagtccagcatcgtgacgaagaagcagctggagactcgcaagcacgcctgccaactccccctgaagggtgagagctcgacccgccagaacacgatggtggagtccagcgacgaggaggatgaggacgagggcattgtcgtgcgcatcgactcccccagcaagatcgtgttccgcaacgacctcaccggtgactacaaggacgacgacgacaagagcggcgagaacctgtactttcagggccacaaccaccgccacaagcacaccggttaa

SEQ ID NO: 23 - DNA, 711 bp, Artificial Sequence
  Codon optimized sequence
atggttcgtggcaatgatatgcttcccaatggccacttccacaagaagtggcagttccacgtgaagacgtggttcaaccagccggcgcgcaagcagaggaggcgcaacgcccgcgctgagaaggccaaggcgaccttcccacgcccggtcgctggctcgctgaagcccatcgtgcgctgccagaccgtcaagtacaacaccaagcagcgcctgggccgtggcttcaccctggaggagctgaaggaggcgggcatccccgccaagtttgcgcccaccgtgggcatcgccgtggaccaccgccgcaagaaccgctctctggagacgctgcaggccaacgtgcagcgtctcaagacgtaccgcgcgtcgctcgtcatcttcccgcgcaacatgaagaagcccaaggcgtttgaggcgtcggctgctgactgctccgccgcgtcgcaggccaagggcgagctgctgccgctcaagggcaccaagcccgcgctggagctggtcaagatcacggccgacatgaaggagggttcccagtacggcaagctgcgcatcgagcgcgtcaacgcacggctcaagggcatgcgcgagaagcgcgccgcggacgaggcggccaagaaggacgacaagaccggtgactacaaggacgacgacgacaagagcggcgagaacctgtactttcagggccacaaccaccgccacaagcacaccggttaa

SEQ ID NO: 24 - DNA, 576 bp, Artificial Sequence
  Codon optimized sequence
atggcgtcgccattctacgcgctcgccgcgaccgacatcgccggcaaggagtttccgttcgcgcagctgcagggaaaagtggtgctcgtcgttaacgtcgcgagccagtgtggcttcacaccccagtacaagggcctccaggagctgtatgacaagtacaaggacgagggcctggtgatcatcggcttcccgtgcgaccagttcggccatcaggagcccggccaggagtctgagatcgccagcttctgccagaagaacttcggcgtgacgttcccgatgatggccaagatcgaggtaaacggcgacaacacgcacccggtctaccagttcctcaagtcggagaagaagcagctgttcatggagcgcatcaagtggaactttgaaaagttcctgatcaacaagcagggcgaggtcgtggagcgcttctcatcggcgggcgacccaatgaggaacatcgccccggcagtcgccaagctcctggccgaggcaaccggtgattataaggatgatgacgacaagagcggtgagaacctgtacttccagggccacaaccaccgccacaagcataccggttaa

SEQ ID NO: 25 - protein, 191 aa, Chlamydomonas
  MISC_FEATURE (164)..(191): FLAG-TEV-MAT tag
MASPFYALAATDIAGKEFPFAQLQGKVVLVVNVASQCGFTPQYKGLQELYDKYKDEGLVIIGFPCDQFGHQEPGQESEIASFCQKNFGVTFPMMAKIEVNGDNTHPVYQFLKSEKKQLFMERIKWNFEKFLINKQGEVVERFSSAGDPMRNIAPAVAKLLAEATGDYKDDDDKSGENLYFQGHNHRHKHTG

SEQ ID NO: 26 - DNA, 489 bp, Artificial Sequence
  Codon optimized sequence
atggcgtcgccattctacgcgctcgccgcgaccgacatcgccggcaaggagtttccgttcgcgcagctgcagggaaaagtggtgctcgtcgttaacgtcgcgagccagtgtggcttcacaccccagtacaagggcctccaggagctgtatgacaagtacaaggacgagggcctggtgatcatcggcttcccgtgcgaccagttcggccatcaggagcccggccaggagtctgagatcgccagcttctgccagaagaacttcggcgtgacgttcccgatgatggccaagatcgaggtaaacggcgacaacacgcacccggtctaccagttcctcaagtcggagaagaagcagctgttcatggagcgcatcaagtggaactttgaaaagttcctgatcaacaagcagggcgaggtcgtggagcgcttctcatcggcgggcgacccaatgaggaacatcgccccggcagtcgccaagctcctggccgaggca

SEQ ID NO: 27 - protein, 163 aa, Chlamydomonas
MASPFYALAATDIAGKEFPFAQLQGKVVLVVNVASQCGFTPQYKGLQELYDKYKDEGLVIIGFPCDQFGHQEPGQESEIASFCQKNFGVTFPMMAKIEVNGDNTHPVYQFLKSEKKQLFMERIKWNFEKFLINKQGEVVERFSSAGDPMRNIAPAVAKLLAEA

SEQ ID NO: 28 - protein, 28 aa, Artificial Sequence
  FLAG-TEV-MAT tag
TGDYKDDDDKSGENLYFQGHNHRHKHTG

SEQ ID NO: 29 - DNA, 573 bp, Artificial Sequence
  Codon optimized sequence
atggcgaaccccgagttttacggcctgtccacgaccacgctttcgggacagccctttcctttcaaggaccttgagggcaaggcagtcctaatcgtgaacgtggcaagcaagtgcggctttacgccacaatacaagggtctggaggagctgtaccaacagtacaaggaccgggggctcgtcatccttggcttcccctgcaaccagtttggaggccaggagcctggggacgcaagcgccatcggggagttctgccagcgcaatttcggcgttacgttccccattatggagaagtcggacgtgaacggcaacgacgccaaccccgtgttcaagtacctgaagagccaaaagaagcagttcatgatggagatgattaaatggaactttgagaagttcctggtggacaagagcggccaggtggtggcgcgcttcagcagcatggctacccccgccagcctggcgccggagattgagaaggtgctgaacgcgaccggtgattataaggatgacgatgacaagagcggtgagaacctgtacttccagggccacaaccaccgccataagcacaccggttag

SEQ ID NO: 30 - protein, 190 aa, Chlamydomonas reinhardtii
  MISC_FEATURE (163)..(190): FLAG-TEV-MAT tag
MANPEFYGLSTTTLSGQPFPFKDLEGKAVLIVNVASKCGFTPQYKGLEELYQQYKDRGLVILGFPCNQFGGQEPGDASAIGEFCQRNFGVTFPIMEKSDVNGNDANPVFKYLKSQKKQFMMEMIKWNFEKFLVDKSGQVVARFSSMATPASLAPEIEKVLNATGDYKDDDDKSGENLYFQGHNHRHKHTG

SEQ ID NO: 31 - DNA, 486 bp, Artificial Sequence
  Codon optimized sequence
atggcgaaccccgagttttacggcctgtccacgaccacgctttcgggacagccctttcctttcaaggaccttgagggcaaggcagtcctaatcgtgaacgtggcaagcaagtgcggctttacgccacaatacaagggtctggaggagctgtaccaacagtacaaggaccgggggctcgtcatccttggcttcccctgcaaccagtttggaggccaggagcctggggacgcaagcgccatcggggagttctgccagcgcaatttcggcgttacgttccccattatggagaagtcggacgtgaacggcaacgacgccaaccccgtgttcaagtacctgaagagccaaaagaagcagttcatgatggagatgattaaatggaactttgagaagttcctggtggacaagagcggccaggtggtggcgcgcttcagcagcatggctacccccgccagcctggcgccggagattgagaaggtgctgaacgcg

SEQ ID NO: 32 - protein, 162 aa, Chlamydomonas reinhardtii
MANPEFYGLSTTTLSGQPFPFKDLEGKAVLIVNVASKCGFTPQYKGLEELYQQYKDRGLVILGFPCNQFGGQEPGDASAIGEFCQRNFGVTFPIMEKSDVNGNDANPVFKYLKSQKKQFMMEMIKWNFEKFLVDKSGQVVARFSSMATPASLAPEIEKVLNA

SEQ ID NO: 33 - DNA, 561 bp, Artificial Sequence
  Codon optimized sequence
atgtcccatttctacgacctggctcctaaggataaggatggtaacccgtttcccttcagcaacctgaagggcaaggtggtgctcgtggtgaacaccgcctccaagtgcggctttacgcctcagtacaaggggctggaggccctgtaccagaagtataaggaccgcggcttcatcatcctgggcttcccgtgcaaccagttcggcaaccaggagccgggctccgacgaggagatcgcccagttctgccaaaagaactacggcgtgaccttcccggtgctggccaagatcaacgtgaacggcgacaacgtggaccctgtctaccagttcctgaagagccagaagaagcagctgggcctggagcgcatcaagtggaacttcgagaagttcctggtgaaccggcaagggcaggtgatcgagcgctacagctccatctcgaagccggagcatctggagaacgacatcgagtcggtcctgaccggtgactataaggacgacgatgacaagtcgggggagaacctgtatttccaggggcacaaccaccgccacaagcacaccggttaa

SEQ ID NO: 34 - protein, 186 aa, Schizosaccharomyces pombe
  MISC_FEATURE (159)..(186): FLAG-TEV-MAT tag
MSHFYDLAPKDKDGNPFPFSNLKGKVVLVVNTASKCGFTPQYKGLEALYQKYKDRGFIILGFPCNQFGNQEPGSDEEIAQFCQKNYGVTFPVLAKINVNGDNVDPVYQFLKSQKKQLGLERIKWNFEKFLVNRQGQVIERYSSISKPEHLENDIESVLTGDYKDDDDKSGENLYFQGHNHRHKHTG

SEQ ID NO: 35 - DNA, 474 bp, Artificial Sequence
  Codon optimized sequence
atgtcccatttctacgacctggctcctaaggataaggatggtaacccgtttcccttcagcaacctgaagggcaaggtggtgctcgtggtgaacaccgcctccaagtgcggctttacgcctcagtacaaggggctggaggccctgtaccagaagtataaggaccgcggcttcatcatcctgggcttcccgtgcaaccagttcggcaaccaggagccgggctccgacgaggagatcgcccagttctgccaaaagaactacggcgtgaccttcccggtgctggccaagatcaacgtgaacggcgacaacgtggaccctgtctaccagttcctgaagagccagaagaagcagctgggcctggagcgcatcaagtggaacttcgagaagttcctggtgaaccggcaagggcaggtgatcgagcgctacagctccatctcgaagccggagcatctggagaacgacatcgagtcggtcctg

SEQ ID NO: 36 - protein, 158 aa, Schizosaccharomyces pombe
MSHFYDLAPKDKDGNPFPFSNLKGKVVLVVNTASKCGFTPQYKGLEALYQKYKDRGFIILGFPCNQFGNQEPGSDEEIAQFCQKNYGVTFPVLAKINVNGDNVDPVYQFLKSQKKQLGLERIKWNFEKFLVNRQGQVIERYSSISKPEHLENDIESVL

SEQ ID NO: 37 - DNA, 1752 bp, Artificial Sequence
  Codon optimized sequence
atgtggagccagctctccagcctgctgtcgggtaagatggacgcgctgaccacctcggaccacgcctccgtggtgtcgatgaacctgttcgtggcgctgctgtgcggttgcattgtgatcgggcacctcctggaggagaaccgctggatgaacgagagcattaccgccctgctgatcggtctcgcgaccggcgtggtgatcctcctgatctcgggcggcaagagcagccacctgctggtgttcagcgaggatctgttcttcatttacctgctgccgcccatcatcttcaacgcgggctttcaggtgaagaagaagcagttctttcgcaacttcatcaccatcgtgctgttcggcgccgtgggcacgctggtgagcttcaccatcatttccctcggcgcgctgagcatcttcaagaagctggacatcggcaccctggagctggccgactacctggcgatcggcgctatcttcgcggccacggactcggtgtgcaccctgcaggtgctcaaccaggacgagactcctctcctgtattccctcgtgttcggcgagggggtggtgaacgacgccacgtccgtggtgctctttaacgccatccaaagcttcgacctgacccgcatcgaccaccgcatcgccctgcagttcatgggcaacttcctgtacctgttcatcgcgtccacgattctgggcgcgttcacgggcctgctgagcgcctacatcattaagaagctgtacttcggccgccactcgaccgaccgcgaggtcgcgctgatgatgctgatggcttacctgagctacatgctggccgagctgttctacctgtcgggcattctcacggtcttcttctgcgggatcgtcatgagccactacacctggcacaacgtgacggagtcgtcccgcgtgaccaccaagcacgctttcgctacgctgagcttcgtcgctgaggtgttcctgtttctgtacgtcggtatggacgccctcgacatcgagaagtggcggtttgtgagcgatagccccggcatctcggtcgccgtgagctcgatcctgctgggcctggtgatggtcgggcgcgcggccttcgtgttccccctgagctggctgatgaacttcgccaagaagagccagtccgagaaggtgacgttcaaccagcagatcgtgatctggtgggcgggcctgatgcgcggggcggtgtccatggcgctggcctacaaccaattcactcgctcgggccacacccagctgcgcggcaacgccatcatgattacttcgacgattagcgtggtgctgttctcgactatggtgttcggcctgctcaccaagcccctgatcatgttcctcctgccgcagcccaagcacttcacctcctgctccaccgtgtccgacgtgggcagcccgaagtcctattcgctccctctgctggagggcaaccaggattacgaggtggacgtcggcaacggcaaccatgaggacacgaccgagccccgcactatcgtgcgcccgtcgagcctgcgcatgctgctgaacgctcccacgcataccgtccaccactactggcggaagttcgacgactccttcatgcgccccgtctttggcggccggggcttcgtccctttcgtcccgggttcccccacggagcagtccaccaacaacctggtggaccggaccaccggtgactacaaggatgacgatgataagtcgggcgagaacctctacttccagggccacaaccaccgccacaagcacaccggttaa

SEQ ID NO: 38 - protein, 583 aa, Atriplex gmelini
  MISC_FEATURE (556)..(583): FLAG-TEV-MAT tag
MWSQLSSLLSGKMDALTTSDHASVVSMNLFVALLCGCIVIGHLLEENRWMNESITALLIGLATGVVILLISGGKSSHLLVFSEDLFFIYLLPPIIFNAGFQVKKKQFFRNFITIVLFGAVGTLVSFTIISLGALSIFKKLDIGTLELADYLAIGAIFAATDSVCTLQVLNQDETPLLYSLVFGEGVVNDATSVVLFNAIQSFDLTRIDHRIALQFMGNFLYLFIASTILGAFTGLLSAYIIKKLYFGRHSTDREVALMMLMAYLSYMLAELFYLSGILTVFFCGIVMSHYTWHNVTESSRVTTKHAFATLSFVAEVFLFLYVGMDALDIEKWRFVSDSPGISVAVSSILLGLVMVGRAAFVFPLSWLMNFAKKSQSEKVTFNQQIVIWWAGLMRGAVSMALAYNQFTRSGHTQLRGNAIMITSTISVVLFSTMVFGLLTKPLIMFLLPQPKHFTSCSTVSDVGSPKSYSLPLLEGNQDYEVDVGNGNHEDTTEPRTIVRPSSLRMLLNAPTHTVHHYWRKFDDSFMRPVFGGRGFVPFVPGSPTEQSTNNLVDRTTGDYKDDDDKSGENLYFQGHNHRHKHTG

SEQ ID NO: 39 - DNA, 1665 bp, Artificial Sequence
  Codon optimized sequence
atgtggagccagctctccagcctgctgtcgggtaagatggacgcgctgaccacctcggaccacgcctccgtggtgtcgatgaacctgttcgtggcgctgctgtgcggttgcattgtgatcgggcacctcctggaggagaaccgctggatgaacgagagcattaccgccctgctgatcggtctcgcgaccggcgtggtgatcctcctgatctcgggcggcaagagcagccacctgctggtgttcagcgaggatctgttcttcatttacctgctgccgcccatcatcttcaacgcgggctttcaggtgaagaagaagcagttctttcgcaacttcatcaccatcgtgctgttcggcgccgtgggcacgctggtgagcttcaccatcatttccctcggcgcgctgagcatcttcaagaagctggacatcggcaccctggagctggccgactacctggcgatcggcgctatcttcgcggccacggactcggtgtgcaccctgcaggtgctcaaccaggacgagactcctctcctgtattccctcgtgttcggcgagggggtggtgaacgacgccacgtccgtggtgctctttaacgccatccaaagcttcgacctgacccgcatcgaccaccgcatcgccctgcagttcatgggcaacttcctgtacctgttcatcgcgtccacgattctgggcgcgttcacgggcctgctgagcgcctacatcattaagaagctgtacttcggccgccactcgaccgaccgcgaggtcgcgctgatgatgctgatggcttacctgagctacatgctggccgagctgttctacctgtcgggcattctcacggtcttcttctgcgggatcgtcatgagccactacacctggcacaacgtgacggagtcgtcccgcgtgaccaccaagcacgctttcgctacgctgagcttcgtcgctgaggtgttcctgtttctgtacgtcggtatggacgccctcgacatcgagaagtggcggtttgtgagcgatagccccggcatctcggtcgccgtgagctcgatcctgctgggcctggtgatggtcgggcgcgcggccttcgtgttccccctgagctggctgatgaacttcgccaagaagagccagtccgagaaggtgacgttcaaccagcagatcgtgatctggtgggcgggcctgatgcgcggggcggtgtccatggcgctggcctacaaccaattcactcgctcgggccacacccagctgcgcggcaacgccatcatgattacttcgacgattagcgtggtgctgttctcgactatggtgttcggcctgctcaccaagcccctgatcatgttcctcctgccgcagcccaagcacttcacctcctgctccaccgtgtccgacgtgggcagcccgaagtcctattcgctccctctgctggagggcaaccaggattacgaggtggacgtcggcaacggcaaccatgaggacacgaccgagccccgcactatcgtgcgcccgtcgagcctgcgcatgctgctgaacgctcccacgcataccgtccaccactactggcggaagttcgacgactccttcatgcgccccgtctttggcggccggggcttcgtccctttcgtcccgggttcccccacggagcagtccaccaacaacctggtggaccggacc

SEQ ID NO: 40 - protein, 555 aa, Atriplex gmelini
MWSQLSSLLSGKMDALTTSDHASVVSMNLFVALLCGCIVIGHLLEENRWMNESITALLIGLATGVVILLISGGKSSHLLVFSEDLFFIYLLPPIIFNAGFQVKKKQFFRNFITIVLFGAVGTLVSFTIISLGALSIFKKLDIGTLELADYLAIGAIFAATDSVCTLQVLNQDETPLLYSLVFGEGVVNDATSVVLFNAIQSFDLTRIDHRIALQFMGNFLYLFIASTILGAFTGLLSAYIIKKLYFGRHSTDREVALMMLMAYLSYMLAELFYLSGILTVFFCGIVMSHYTWHNVTESSRVTTKHAFATLSFVAEVFLFLYVGMDALDIEKWRFVSDSPGISVAVSSILLGLVMVGRAAFVFPLSWLMNFAKKSQSEKVTFNQQIVIWWAGLMRGAVSMALAYNQFTRSGHTQLRGNAIMITSTISVVLFSTMVFGLLTKPLIMFLLPQPKHFTSCSTVSDVGSPKSYSLPLLEGNQDYEVDVGNGNHEDTTEPRTIVRPSSLRMLLNAPTHTVHHYWRKFDDSFMRPVFGGRGFVPFVPGSPTEQSTNNLVDRT

SEQ ID NO: 41 - DNA, 1701 bp, Artificial Sequence
  Codon optimized sequence
atgctggattccctggtgagcaagctgccttcgctgtccacctcggaccacgccagcgtggtggccctgaacctcttcgtcgccctgctgtgcgcgtgcatcgtcctgggccacctgctggaggagaaccgctggatgaacgagagcatcacggcgctgctgatcggcctcgggacgggcgtcacgatcctgctgatctccaagggtaagagctcgcacctcctggtcttctcggaggacctcttcttcatctatctgctgccgccgatcatcttcaacgcgggcttccaggtgaagaagaagcaattcttccgcaacttcgtgacgattatgctgttcggcgcggtggggaccatcatctcctgcacgatcatttcgctgggcgtgacgcagttcttcaagaagctcgacatcggcaccttcgacctgggcgactacctggcgatcggtgccatcttcgccgcgaccgactccgtgtgcaccctgcaggtgctgaaccaggacgagacgcccctgctgtactcgctggtgtttggcgagggcgtggtgaacgatgccacctcggtggtggtgttcaacgctatccagtcgttcgacctgactcacctgaaccacgaggccgcgttccatctgctcgggaactttctgtacctgttcctgctcagcaccctgctgggcgcggctacgggcctgatcagcgcgtacgtgattaagaagctgtacttcggccgccacagcacggaccgggaggtggccctcatgatgctgatggcttacctgagctacatgctggccgagctgttcgacctcagcggcatcctgacggtgtttttctgcggcattgtgatgtcgcactacacctggcacaacgtcaccgagtcgtcgcggatcaccactaagcacacctttgccaccctgagctttctggccgagaccttcatcttcctgtacgtgggcatggacgcgctggacatcgataagtggcgcagcgtcagcgacacccccggcaccagcatcgccgtgagctcgatcctcatgggcctggtcatggtgggccgcgccgcgttcgtcttcccgctgagcttcctgtcgaacctggcgaagaagaaccagtcggagaagatcaacttcaacatgcaggtggtgatttggtggagcggcctcatgcggggcgccgtgtccatggctctcgcgtacaacaagttcacccgcgccggccacaccgacgtgcgcggcaacgcgattatgattaccagcacgatcaccgtgtgcctgttctcgaccgtggtctttgggatgctgactaagcctctgatctcctacctgctgccccatcagaacgccacgacctccatgctgtccgacgacaacaccccgaagtccatccacatccccctcctggaccaggattccttcatcgagccctccggcaaccacaacgtgccccgccccgacagcattcggggcttcctgactcgcccgacccggaccgtgcactactactggcgccaattcgacgacagctttatgcgcccggtgttcggtggtcgcgggttcgtgcccttcgtccccggctccccgacggagcgcaacccgcctgacctgtccaaggctaccggtgactacaaggacgacgacgacaagagcggcgagaacctgtacttccagggccacaaccaccgccacaagcacaccggttaa

SEQ ID NO: 42 - protein, 566 aa, Arabidopsis thaliana
  MISC_FEATURE (539)..(566): FLAG-TEV-MAT tag
MLDSLVSKLPSLSTSDHASVVALNLFVALLCACIVLGHLLEENRWMNESITALLIGLGTGVTILLISKGKSSHLLVFSEDLFFIYLLPPIIFNAGFQVKKKQFFRNFVTIMLFGAVGTIISCTIISLGVTQFFKKLDIGTFDLGDYLAIGAIFAATDSVCTLQVLNQDETPLLYSLVFGEGVVNDATSVVVFNAIQSFDLTHLNHEAAFHLLGNFLYLFLLSTLLGAATGLISAYVIKKLYFGRHSTDREVALMMLMAYLSYMLAELFDLSGILTVFFCGIVMSHYTWHNVTESSRITTKHTFATLSFLAETFIFLYVGMDALDIDKWRSVSDTPGTSIAVSSILMGLVMVGRAAFVFPLSFLSNLAKKNQSEKINFNMQVVIWWSGLMRGAVSMALAYNKFTRAGHTDVRGNAIMITSTITVCLFSTVVFGMLTKPLISYLLPHQNATTSMLSDDNTPKSIHIPLLDQDSFIEPSGNHNVPRPDSIRGFLTRPTRTVHYYWRQFDDSFMRPVFGGRGFVPFVPGSPTERNPPDLSKATGDYKDDDDKSGENLYFQGHNHRHKHTG

SEQ ID NO: 43 - DNA, 1614 bp, Artificial Sequence
  Codon optimized sequence
atgctggattccctggtgagcaagctgccttcgctgtccacctcggaccacgccagcgtggtggccctgaacctcttcgtcgccctgctgtgcgcgtgcatcgtcctgggccacctgctggaggagaaccgctggatgaacgagagcatcacggcgctgctgatcggcctcgggacgggcgtcacgatcctgctgatctccaagggtaagagctcgcacctcctggtcttctcggaggacctcttcttcatctatctgctgccgccgatcatcttcaacgcgggcttccaggtgaagaagaagcaattcttccgcaacttcgtgacgattatgctgttcggcgcggtggggaccatcatctcctgcacgatcatttcgctgggcgtgacgcagttcttcaagaagctcgacatcggcaccttcgacctgggcgactacctggcgatcggtgccatcttcgccgcgaccgactccgtgtgcaccctgcaggtgctgaaccaggacgagacgcccctgctgtactcgctggtgtttggcgagggcgtggtgaacgatgccacctcggtggtggtgttcaacgctatccagtcgttcgacctgactcacctgaaccacgaggccgcgttccatctgctcgggaactttctgtacctgttcctgctcagcaccctgctgggcgcggctacgggcctgatcagcgcgtacgtgattaagaagctgtacttcggccgccacagcacggaccgggaggtggccctcatgatgctgatggcttacctgagctacatgctggccgagctgttcgacctcagcggcatcctgacggtgtttttctgcggcattgtgatgtcgcactacacctggcacaacgtcaccgagtcgtcgcggatcaccactaagcacacctttgccaccctgagctttctggccgagaccttcatcttcctgtacgtgggcatggacgcgctggacatcgataagtggcgcagcgtcagcgacacccccggcaccagcatcgccgtgagctcgatcctcatgggcctggtcatggtgggccgcgccgcgttcgtcttcccgctgagcttcctgtcgaacctggcgaagaagaaccagtcggagaagatcaacttcaacatgcaggtggtgatttggtggagcggcctcatgcggggcgccgtgtccatggctctcgcgtacaacaagttcacccgcgccggccacaccgacgtgcgcggcaacgcgattatgattaccagcacgatcaccgtgtgcctgttctcgaccgtggtctttgggatgctgactaagcctctgatctcctacctgctgccccatcagaacgccacgacctccatgctgtccgacgacaacaccccgaagtccatccacatccccctcctggaccaggattccttcatcgagccctccggcaaccacaacgtgccccgccccgacagcattcggggcttcctgactcgcccgacccggaccgtgcactactactggcgccaattcgacgacagctttatgcgcccggtgttcggtggtcgcgggttcgtgcccttcgtccccggctccccgacggagcgcaacccgcctgacctgtccaaggct

SEQ ID NO: 44 - protein, 538 aa, Arabidopsis thaliana
MLDSLVSKLPSLSTSDHASVVALNLFVALLCACIVLGHLLEENRWMNESITALLIGLGTGVTILLISKGKSSHLLVFSEDLFFIYLLPPIIFNAGFQVKKKQFFRNFVTIMLFGAVGTIISCTIISLGVTQFFKKLDIGTFDLGDYLAIGAIFAATDSVCTLQVLNQDETPLLYSLVFGEGVVNDATSVVVFNAIQSFDLTHLNHEAAFHLLGNFLYLFLLSTLLGAATGLISAYVIKKLYFGRHSTDREVALMMLMAYLSYMLAELFDLSGILTVFFCGIVMSHYTWHNVTESSRITTKHTFATLSFLAETFIFLYVGMDALDIDKWRSVSDTPGTSIAVSSILMGLVMVGRAAFVFPLSFLSNLAKKNQSEKINFNMQVVIWWSGLMRGAVSMALAYNKFTRAGHTDVRGNAIMITSTITVCLFSTVVFGMLTKPLISYLLPHQNATTSMLSDDNTPKSIHIPLLDQDSFIEPSGNHNVPRPDSIRGFLTRPTRTVHYYWRQFDDSFMRPVFGGRGFVPFVPGSPTERNPPDLSKA

SEQ ID NO: 45 - DNA, 3525 bp, Artificial Sequence
  Codon optimized sequence
atgaccaccgtgattgatgctaccatggcttatcgctttctggaggaggccactgacagctcgtcgtccagcagctcgtccaagctggagtcctcccccgtggacgctgtcctgttcgtgggcatgtcgctcgtgctcgggatcgcgtcccggcacctgctgcgcgggactcgcgtgccgtacaccgtggccctcctggtgatcggcattgccctgggctccctggagtacggcgccaagcacaacctgggcaagatcggccacggcatccggatctggaacgagattgacccggagctgctgctcgccgtgttcctgccggccctgctgtttgagtcgagcttcagcatggaggtgcaccagatcaagcgctgcctgggccagatggtcctgctggccgtgccgggtgtgctgatctcgaccgcttgcctgggctcgctcgtcaaggtgaccttcccctacgagtgggactggaagacgtcgctgctgctgggcggcctgctgagcgccaccgacccggtggccgtggtggcgctgctgaaggagctcggtgcgagcaagaagctgagcaccatcatcgagggcgagagcctgatgaacgacggcactgcgatcgtggtgttccagctgttcctgaagatggcgatggggcagaactccgactggtcgagcatcatcaagttcctgctgaaggtggctctcggcgccgtgggcatcggtctcgccttcggcatcgcctcggtcatctggctgaagttcatcttcaacgacacggtgatcgagattacgctgacgattgcggtgtcgtactttgcgtactacaccgcgcaggagtgggccggtgcgtcgggcgtgctgaccgtcatgacgctgggcatgttctacgcggcgttcgcccggacggcgttcaagggcgacagccagaagagcctgcaccacttctgggagatggtggcctacatcgcgaacaccctcatcttcatcctgagcggcgtggtgattgcggagggcatcctcgactccgacaagatcgcctaccagggcaactcgtggcgcttcctgttcctcctgtacgtgtacattcagctgagccgcgtggtggtcgtgggcgtgctctacccgctgctgtgccgctttggctacggcctggactggaaggagtccatcatcctggtgtggagcggcctgcgcggcgccgtcgctctggccctgtcgctgtccgtgaagcagagctcgggtaactcgcacatctccaaggagaccggcaccctgttcctgttcttcaccggcggtattgtcttcctgacgctcatcgtgaacggcagcaccacccagttcgtgctgcgcctgctgcgcatggacatcctgcctgcccccaagaagcgcatcctggagtataccaagtacgagatgctgaacaaggcgctgcgcgcttttcaggacctgggtgacgatgaggagctgggccccgccgattggcccacggtggagagctacatctcgtccctgaaggggagcgagggggagctggtccaccacccccacaacggctcgaagatcggcagcctggaccccaagtcgctcaaggacatccggatgcgctttctgaacggcgtccaggcgacctactgggagatgctggacgaggggcgcatcagcgaggtgaccgccaacatcctcatgcagtccgtggacgaggcgctggaccaggtcagcaccaccctgtgcgattggcgcggtctgaagccccatgtcaacttccctaactactacaacttcctgcactcgaaggtggtgccgcggaagctggtcacctacttcgccgtggagcgcctggagtccgcgtgctacatttcggcggctttcctgcgcgctcacaccatcgcccggcagcagctgtacgacttcctcggcgagtccaacatcggcagcattgtcatcaacgagtcggagaaggagggcgaggaggcgaagaagtttctggagaaggtccggagctccttcccgcaggtgctgcgcgtggtgaagactaagcaggtgacgtacagcgtgctgaaccatctgctgggctacatcgagaacctggagaaggtgggcctcctggaggagaaggagatcgcgcacctgcacgacgccgtgcagaccgggctgaagaagctgctgcgcaacccgcctatcgtgaagctgcccaagctgtcggacatgatcacctcccaccccctgtcggtcgccctgccgccggcgttctgcgagcccctgaagcatagcaagaaggagcccatgaagctgcgcggcgtgacgctgtacaaggaggggagcaagcctacgggcgtctggctgatcttcgacggcatcgtgaagtggaagagcaagatcctgtcgaacaaccactccctccacccgacgttctcgcacgggtccacgctggggctctacgaggtcctgacgggcaagccgtacctgtgcgacctcatcacggactccatggtgctgtgcttcttcattgattccgagaagatcctcagcctgcaatcggacagcactatcgacgatttcctgtggcaggagtcggccctggtgctgctcaagctgctccggcctcagatcttcgagtccgtcgcgatgcaggagctgcgcgcgctcgtgtccacggagagctccaagctgaccacgtacgtgaccggcgagtcgatcgagatcgactgcaactcgatcggcctcctgctggagggcttcgtcaagcccgtcggcatcaaggaggagctgattagctcccccgccgccctgtccccctcgaacggcaaccagtcctttcacaacagctccgaggccagcggcatcatgcgcgtgtcgttcagccagcaggcgacccaatacatcgtggagacgcgggcccgggctatcattttcaacattggcgccttcggtgccgaccgcaccctgcaccgccgcccctccagcctgaccccgccccgctcgtccagctccgaccagctgcagcggagcttccgcaaggagcaccgcgggctgatgtcgtggcccgagaacatctacgcgaagcagcagcaggagattaacaagaccacgctgtccctgtcggagcgcgccatgcagctgagcattttcggctcgatggtgaacgtgtatcgccggagcgtgagcttcggcggcatctacaacaacaagctgcaggacaacctgctgtataagaagctgcctctgaaccccgcgcagggcctcgtgagcgccaagagcgagtccagcatcgtgacgaagaagcagctggagactcgcaagcacgcctgccaactccccctgaagggtgagagctcgacccgccagaacacgatggtggagtccagcgacgaggaggatgaggacgagggcattgtcgtgcgcatcgactcccccagcaagatcgtgttccgcaacgacctcaccggtgactacaaggacgacgacgacaagagcggcgagaacctgtactttcagggtcacaaccaccgccacaagcacaccggttag

SEQ ID NO: 46 - protein, 1174 aa, Arabidopsis thaliana
  MISC_FEATURE (1147)..(1174): FLAG-TEV-MAT tag
MTTVIDATMAYRFLEEATDSSSSSSSSKLESSPVDAVLFVGMSLVLGIASRHLLRGTRVPYTVALLVIGIALGSLEYGAKHNLGKIGHGIRIWNEIDPELLLAVFLPALLFESSFSMEVHQIKRCLGQMVLLAVPGVLISTACLGSLVKVTFPYEWDWKTSLLLGGLLSATDPVAVVALLKELGASKKLSTIIEGESLMNDGTAIVVFQLFLKMAMGQNSDWSSIIKFLLKVALGAVGIGLAFGIASVIWLKFIFNDTVIEITLTIAVSYFAYYTAQEWAGASGVLTVMTLGMFYAAFARTAFKGDSQKSLHHFWEMVAYIANTLIFILSGVVIAEGILDSDKIAYQGNSWRFLFLLYVYIQLSRVVVVGVLYPLLCRFGYGLDWKESIILVWSGLRGAVALALSLSVKQSSGNSHISKETGTLFLFFTGGIVFLTLIVNGSTTQFVLRLLRMDILPAPKKRILEYTKYEMLNKALRAFQDLGDDEELGPADWPTVESYISSLKGSEGELVHHPHNGSKIGSLDPKSLKDIRMRFLNGVQATYWEMLDEGRISEVTANILMQSVDEALDQVSTTLCDWRGLKPHVNFPNYYNFLHSKVVPRKLVTYFAVERLESACYISAAFLRAHTIARQQLYDFLGESNIGSIVINESEKEGEEAKKFLEKVRSSFPQVLRVVKTKQVTYSVLNHLLGYIENLEKVGLLEEKEIAHLHDAVQTGLKKLLRNPPIVKLPKLSDMITSHPLSVALPPAFCEPLKHSKKEPMKLRGVTLYKEGSKPTGVWLIFDGIVKWKSKILSNNHSLHPTFSHGSTLGLYEVLTGKPYLCDLITDSMVLCFFIDSEKILSLQSDSTIDDFLWQESALVLLKLLRPQIFESVAMQELRALVSTESSKLTTYVTGESIEIDCNSIGLLLEGFVKPVGIKEELISSPAALSPSNGNQSFHNSSEASGIMRVSFSQQATQYIVETRARAIIFNIGAFGADRTLHRRPSSLTPPRSSSSDQLQRSFRKEHRGLMSWPENIYAKQQQEINKTTLSLSERAMQLSIFGSMVNVYRRSVSFGGIYNNKLQDNLLYKKLPLNPAQGLVSAKSESSIVTKKQLETRKHACQLPLKGESSTRQNTMVESSDEEDEDEGIVVRIDSPSKIVFRNDLTGDYKDDDDKSGENLYFQGHNHRHKHTG

SEQ ID NO: 47 - DNA, 3438 bp, Artificial Sequence
  Codon optimized sequence
atgaccaccgtgattgatgctaccatggcttatcgctttctggaggaggccactgacagctcgtcgtccagcagctcgtccaagctggagtcctcccccgtggacgctgtcctgttcgtgggcatgtcgctcgtgctcgggatcgcgtcccggcacctgctgcgcgggactcgcgtgccgtacaccgtggccctcctggtgatcggcattgccctgggctccctggagtacggcgccaagcacaacctgggcaagatcggccacggcatccggatctggaacgagattgacccggagctgctgctcgccgtgttcctgccggccctgctgtttgagtcgagcttcagcatggaggtgcaccagatcaagcgctgcctgggccagatggtcctgctggccgtgccgggtgtgctgatctcgaccgcttgcctgggctcgctcgtcaaggtgaccttcccctacgagtgggactggaagacgtcgctgctgctgggcggcctgctgagcgccaccgacccggtggccgtggtggcgctgctgaaggagctcggtgcgagcaagaagctgagcaccatcatcgagggcgagagcctgatgaacgacggcactgcgatcgtggtgttccagctgttcctgaagatggcgatggggcagaactccgactggtcgagcatcatcaagttcctgctgaaggtggctctcggcgccgtgggcatcggtctcgccttcggcatcgcctcggtcatctggctgaagttcatcttcaacgacacggtgatcgagattacgctgacgattgcggtgtcgtactttgcgtactacaccgcgcaggagtgggccggtgcgtcgggcgtgctgaccgtcatgacgctgggcatgttctacgcggcgttcgcccggacggcgttcaagggcgacagccagaagagcctgcaccacttctgggagatggtggcctacatcgcgaacaccctcatcttcatcctgagcggcgtggtgattgcggagggcatcctcgactccgacaagatcgcctaccagggcaactcgtggcgcttcctgttcctcctgtacgtgtacattcagctgagccgcgtggtggtcgtgggcgtgctctacccgctgctgtgccgctttggctacggcctggactggaaggagtccatcatcctggtgtggagcggcctgcgcggcgccgtcgctctggccctgtcgctgtccgtgaagcagagctcgggtaactcgcacatctccaaggagaccggcaccctgttcctgttcttcaccggcggtattgtcttcctgacgctcatcgtgaacggcagcaccacccagttcgtgctgcgcctgctgcgcatggacatcctgcctgcccccaagaagcgcatcctggagtataccaagtacgagatgctgaacaaggcgctgcgcgcttttcaggacctgggtgacgatgaggagctgggccccgccgattggcccacggtggagagctacatctcgtccctgaaggggagcgagggggagctggtccaccacccccacaacggctcgaagatcggcagcctggaccccaagtcgctcaaggacatccggatgcgctttctgaacggcgtccaggcgacctactgggagatgctggacgaggggcgcatcagcgaggtgaccgccaacatcctcatgcagtccgtggacgaggcgctggaccaggtcagcaccaccctgtgcgattggcgcggtctgaagccccatgtcaacttccctaactactacaacttcctgcactcgaaggtggtgccgcggaagctggtcacctacttcgccgtggagcgcctggagtccgcgtgctacatttcggcggctttcctgcgcgctcacaccatcgcccggcagcagctgtacgacttcctcggcgagtccaacatcggcagcattgtcatcaacgagtcggagaaggagggcgaggaggcgaagaagtttctggagaaggtccggagctccttcccgcaggtgctgcgcgtggtgaagactaagcaggtgacgtacagcgtgctgaaccatctgctgggctacatcgagaacctggagaaggtgggcctcctggaggagaaggagatcgcgcacctgcacgacgccgtgcagaccgggctgaagaagctgctgcgcaacccgcctatcgtgaagctgcccaagctgtcggacatgatcacctcccaccccctgtcggtcgccctgccgccggcgttctgcgagcccctgaagcatagcaagaaggagcccatgaagctgcgcggcgtgacgctgtacaaggaggggagcaagcctacgggcgtctggctgatcttcgacggcatcgtgaagtggaagagcaagatcctgtcgaacaaccactccctccacccgacgttctcgcacgggtccacgctggggctctacgaggtcctgacgggcaagccgtacctgtgcgacctcatcacggactccatggtgctgtgcttcttcattgattccgagaagatcctcagcctgcaatcggacagcactatcgacgatttcctgtggcaggagtcggccctggtgctgctcaagctgctccggcctcagatcttcgagtccgtcgcgatgcaggagctgcgcgcgctcgtgtccacggagagctccaagctgaccacgtacgtgaccggcgagtcgatcgagatcgactgcaactcgatcggcctcctgctggagggcttcgtcaagcccgtcggcatcaaggaggagctgattagctcccccgccgccctgtccccctcgaacggcaaccagtcctttcacaacagctccgaggccagcggcatcatgcgcgtgtcgttcagccagcaggcgacccaatacatcgtggagacgcgggcccgggctatcattttcaacattggcgccttcggtgccgaccgcaccctgcaccgccgcccctccagcctgaccccgccccgctcgtccagctccgaccagctgcagcggagcttccgcaaggagcaccgcgggctgatgtcgtggcccgagaacatctacgcgaagcagcagcaggagattaacaagaccacgctgtccctgtcggagcgcgccatgcagctgagcattttcggctcgatggtgaacgtgtatcgccggagcgtgagcttcggcggcatctacaacaacaagctgcaggacaacctgctgtataagaagctgcctctgaaccccgcgcagggcctcgtgagcgccaagagcgagtccagcatcgtgacgaagaagcagctggagactcgcaagcacgcctgccaactccccctgaagggtgagagctcgacccgccagaacacgatggtggagtccagcgacgaggaggatgaggacgagggcattgtcgtgcgcatcgactcccccagcaagatcgtgttccgcaacgacctc

SEQ ID NO: 48 - protein, 1146 aa, Arabidopsis thaliana
MTTVIDATMAYRFLEEATDSSSSSSSSKLESSPVDAVLFVGMSLVLGIASRHLLRGTRVPYTVALLVIGIALGSLEYGAKHNLGKIGHGIRIWNEIDPELLLAVFLPALLFESSFSMEVHQIKRCLGQMVLLAVPGVLISTACLGSLVKVTFPYEWDWKTSLLLGGLLSATDPVAVVALLKELGASKKLSTIIEGESLMNDGTAIVVFQLFLKMAMGQNSDWSSIIKFLLKVALGAVGIGLAFGIASVIWLKFIFNDTVIEITLTIAVSYFAYYTAQEWAGASGVLTVMTLGMFYAAFARTAFKGDSQKSLHHFWEMVAYIANTLIFILSGVVIAEGILDSDKIAYQGNSWRFLFLLYVYIQLSRVVVVGVLYPLLCRFGYGLDWKESIILVWSGLRGAVALALSLSVKQSSGNSHISKETGTLFLFFTGGIVFLTLIVNGSTTQFVLRLLRMDILPAPKKRILEYTKYEMLNKALRAFQDLGDDEELGPADWPTVESYISSLKGSEGELVHHPHNGSKIGSLDPKSLKDIRMRFLNGVQATYWEMLDEGRISEVTANILMQSVDEALDQVSTTLCDWRGLKPHVNFPNYYNFLHSKVVPRKLVTYFAVERLESACYISAAFLRAHTIARQQLYDFLGESNIGSIVINESEKEGEEAKKFLEKVRSSFPQVLRVVKTKQVTYSVLNHLLGYIENLEKVGLLEEKEIAHLHDAVQTGLKKLLRNPPIVKLPKLSDMITSHPLSVALPPAFCEPLKHSKKEPMKLRGVTLYKEGSKPTGVWLIFDGIVKWKSKILSNNHSLHPTFSHGSTLGLYEVLTGKPYLCDLITDSMVLCFFIDSEKILSLQSDSTIDDFLWQESALVLLKLLRPQIFESVAMQELRALVSTESSKLTTYVTGESIEIDCNSIGLLLEGFVKPVGIKEELISSPAALSPSNGNQSFHNSSEASGIMRVSFSQQATQYIVETRARAIIFNIGAFGADRTLHRRPSSLTPPRSSSSDQLQRSFRKEHRGLMSWPENIYAKQQQEINKTTLSLSERAMQLSIFGSMVNVYRRSVSFGGIYNNKLQDNLLYKKLPLNPAQGLVSAKSESSIVTKKQLETRKHACQLPLKGESSTRQNTMVESSDEEDEDEGIVVRIDSPSKIVFRNDL

SEQ ID NO: 49 - DNA, 711 bp, Artificial Sequence
  Codon optimized sequence
atggttcgtggcaatgatatgcttcccaatggccacttccacaagaagtggcagttccacgtgaagacgtggttcaaccagccggcgcgcaagcagaggaggcgcaacgcccgcgctgagaaggccaaggcgaccttcccacgcccggtcgctggctcgctgaagcccatcgtgcgctgccagaccgtcaagtacaacaccaagcagcgcctgggccgtggcttcaccctggaggagctgaaggaggcgggcatccccgccaagtttgcgcccaccgtgggcatcgccgtggaccaccgccgcaagaaccgctctctggagacgctgcaggccaacgtgcagcgtctcaagacgtaccgcgcgtcgctcgtcatcttcccgcgcaacatgaagaagcccaaggcgtttgaggcgtcggctgctgactgctccgccgcgtcgcaggccaagggcgagctgctgccgctcaagggcaccaagcccgcgctggagctggtcaagatcacggccgacatgaaggagggttcccagtacggcaagctgcgcatcgagcgcgtcaacgcacggctcaagggcatgcgcgagaagcgcgccgcggacgaggcggccaagaaggacgacaagaccggtgattataaggacgacgacgataagagcggcgagaacctgtacttccaagggcacaaccaccggcacaagcacaccggttaa

SEQ ID NO: 50 - protein, 236 aa, Chlamydomonas
  MISC_FEATURE (209)..(236): FLAG-TEV-MAT tag
MVRGNDMLPNGHFHKKWQFHVKTWFNQPARKQRRRNARAEKAKATFPRPVAGSLKPIVRCQTVKYNTKQRLGRGFTLEELKEAGIPAKFAPTVGIAVDHRRKNRSLETLQANVQRLKTYRASLVIFPRNMKKPKAFEASAADCSAASQAKGELLPLKGTKPALELVKITADMKEGSQYGKLRIERVNARLKGMREKRAADEAAKKDDKTGDYKDDDDKSGENLYFQGHNHRHKHTG

SEQ ID NO: 51 - DNA, 624 bp, Artificial Sequence
  Codon optimized sequence
atggttcgtggcaatgatatgcttcccaatggccacttccacaagaagtggcagttccacgtgaagacgtggttcaaccagccggcgcgcaagcagaggaggcgcaacgcccgcgctgagaaggccaaggcgaccttcccacgcccggtcgctggctcgctgaagcccatcgtgcgctgccagaccgtcaagtacaacaccaagcagcgcctgggccgtggcttcaccctggaggagctgaaggaggcgggcatccccgccaagtttgcgcccaccgtgggcatcgccgtggaccaccgccgcaagaaccgctctctggagacgctgcaggccaacgtgcagcgtctcaagacgtaccgcgcgtcgctcgtcatcttcccgcgcaacatgaagaagcccaaggcgtttgaggcgtcggctgctgactgctccgccgcgtcgcaggccaagggcgagctgctgccgctcaagggcaccaagcccgcgctggagctggtcaagatcacggccgacatgaaggagggttcccagtacggcaagctgcgcatcgagcgcgtcaacgcacggctcaagggcatgcgcgagaagcgcgccgcggacgaggcggccaagaaggacgacaag

SEQ ID NO: 52 - protein, 208 aa, Chlamydomonas
MVRGNDMLPNGHFHKKWQFHVKTWFNQPARKQRRRNARAEKAKATFPRPVAGSLKPIVRCQTVKYNTKQRLGRGFTLEELKEAGIPAKFAPTVGIAVDHRRKNRSLETLQANVQRLKTYRASLVIFPRNMKKPKAFEASAADCSAASQAKGELLPLKGTKPALELVKITADMKEGSQYGKLRIERVNARLKGMREKRAADEAAKKDDK

SEQ ID NO: 53 - DNA, 876 bp, Artificial Sequence
  Codon optimized sequence
atgtccgccgacgcggagaagcagtccctgctggccacgggcgtgccggcccacgccgctggggacgcgccgaaggtggcgccccgcgagtggcgccatcgctggtacgcgatcctcggtgattgctcggcccccgacgtggtctcgtgcctcctggcctggaagctgcccttcgtcgcgtgggcctggaaccagaaccgcgccctgggcatgagcttctggcgcgagctgctgcgcttcgcggtcatcgtggtgggctttgtggtggcgacgcacgtggcgtactgcggcgtcatgatggctatgtgccccgagatccacgaccgggacggcgctagcgtggacggcggccccggcatgatgcgcaagctgctgcacatgcaccagcaccactcccaccaccatgacgacgacagcaccgacgactcgacggacagccacgaccacggcatgtggggcgaggatggcccccacggcatccctcgcgagtgcgtggcgcgcgtggccccggcctacgtggccattacgggtgtgttcctggccctggcggtgtacatgaccctcttcttcgctcggcgccgcacggcgctgcgcgagcgctacggcattgccgggaccgcccgggaggattgcctgctgtacgccttctgcaccccttgcgcgctggctcaggagacccggactctgatccacgagcaggtgcacgacggtatctggtatggcgcgctgccgggcgtcgcgccgcctgccgcgaccgtcgccgcgcccgcgccccaaaagatggccgtgaccggtgactacaaggacgacgacgacaagagcggcgagaacctgtactttcagggccacaaccaccgccacaagcacaccggttaa

SEQ ID NO: 54 - protein, 291 aa, Chlamydomonas
  MISC_FEATURE (264)..(291): FLAG-TEV-MAT tag
MSADAEKQSLLATGVPAHAAGDAPKVAPREWRHRWYAILGDCSAPDVVSCLLAWKLPFVAWAWNQNRALGMSFWRELLRFAVIVVGFVVATHVAYCGVMMAMCPEIHDRDGASVDGGPGMMRKLLHMHQHHSHHHDDDSTDDSTDSHDHGMWGEDGPHGIPRECVARVAPAYVAITGVFLALAVYMTLFFARRRTALRERYGIAGTAREDCLLYAFCTPCALAQETRTLIHEQVHDGIWYGALPGVAPPAATVAAPAPQKMAVTGDYKDDDDKSGENLYFQGHNHRHKHTG

SEQ ID NO: 55 - DNA, 789 bp, Artificial Sequence
  Codon optimized sequence
atgtccgccgacgcggagaagcagtccctgctggccacgggcgtgccggcccacgccgctggggacgcgccgaaggtggcgccccgcgagtggcgccatcgctggtacgcgatcctcggtgattgctcggcccccgacgtggtctcgtgcctcctggcctggaagctgcccttcgtcgcgtgggcctggaaccagaaccgcgccctgggcatgagcttctggcgcgagctgctgcgcttcgcggtcatcgtggtgggctttgtggtggcgacgcacgtggcgtactgcggcgtcatgatggctatgtgccccgagatccacgaccgggacggcgctagcgtggacggcggccccggcatgatgcgcaagctgctgcacatgcaccagcaccactcccaccaccatgacgacgacagcaccgacgactcgacggacagccacgaccacggcatgtggggcgaggatggcccccacggcatccctcgcgagtgcgtggcgcgcgtggccccggcctacgtggccattacgggtgtgttcctggccctggcggtgtacatgaccctcttcttcgctcggcgccgcacggcgctgcgcgagcgctacggcattgccgggaccgcccgggaggattgcctgctgtacgccttctgcaccccttgcgcgctggctcaggagacccggactctgatccacgagcaggtgcacgacggtatctggtatggcgcgctgccgggcgtcgcgccgcctgccgcgaccgtcgccgcgcccgcgccccaaaagatggccgtg

SEQ ID NO: 56 - protein, 263 aa, Chlamydomonas
MSADAEKQSLLATGVPAHAAGDAPKVAPREWRHRWYAILGDCSAPDVVSCLLAWKLPFVAWAWNQNRALGMSFWRELLRFAVIVVGFVVATHVAYCGVMMAMCPEIHDRDGASVDGGPGMMRKLLHMHQHHSHHHDDDSTDDSTDSHDHGMWGEDGPHGIPRECVARVAPAYVAITGVFLALAVYMTLFFARRRTALRERYGIAGTAREDCLLYAFCTPCALAQETRTLIHEQVHDGIWYGALPGVAPPAATVAAPAPQKMAV

SEQ ID NO: 57 - DNA, 2199 bp, Chlamydomonas reinhardtii
atggcgctcacgaacgccattatcgctgcaacccgtgcacaagagcaaaattttgatgaggactcagggcgaatgcgcgaaatcggcgcggcccgcgaggcccgcggcgagggccttgccaaaggccccggcggtggaggaggatatggcagcagtggtggaggtttcggcatcgtgcatgctaggactgcctcagaggtggcgagtaccggtccgtcacagtgggcgctactgcaagagcgcattaccgcagctcagggctccgtgcccgacccggcctccgacgacgtggccaggttgatgcgatccatcttcatgcagcacctaatgtctggcgcgcctgagtactcaaagtacttcaagaatgacatacgcatgatgcagcaagaggcggagttgcagaaacaggcggccaaggaggcagaggcctcggcatcggggcaccgccgcatgtccacggcgggcggcagcgccggcggcgcttccgatgccgccggcagcccatacagcgcttccgcaggcaggacagcatcgcagccgcaattgcgcccggaccaccaccacaacgacccgccgcccaacatattcgcgtctttgtaccgcccctgcaagcacgcgctggcgcggtatcgcgcatcgccgcttcgggccaagatatacctgacgctctcgcaccccgagtacaacgcagtcgcattcacgttcggcatcttcgtcatgcttgtcatcctgctgaacacggctgtgttctgcattgagagtgtgccgcgctgggagaacacgccgctgtacgaccggcttgtcatcgtggactacgtgtgcctgggtatcttcacggtggagttcgtggcgcggctggtgacgtgcagcagcctgacgcacttctggctcaacgcaatgaactggatcgacttcttcgccatcgcgcccttctacctcgagctcatgatcgtggggccggacgcaggaaaccaggctgcctcccagacgcgcatcatccgggtgctgcgcctgctgcgtgtgctgcgcctcatgcgcgcctccaccaggttccgcaacctgcaggtggtggtggacgcgctggtggccagtggcgacgtgctgggcatgctggtgttcctgctgctggtgctgctggtggtgtcggccaccatcatctactttgtggagcaggcgctggtggaggggtcctggttcgactccataccgctcaccatctactacatgcacgtgacactgaccaccaccggctacggcgacttctatcccgtgtcggcctggggccgcttcatcgcgggcgtgttcatgctgttgtgcatggtgacgctatcgctgcccatcagcgtgatcggcggcaacttctccaacatgtggggccgctacacacatatccgcgacggcattgagcgcagcggcgtggcctggagcaacttcatcaagctgcgcggcaccgccacgaaacactgcgcggccatggacgacctcattgacatcataaaccgcgtcaagtgcgcgctggaagacggcacccgcggcggcggtgctgtgggccagccgggcgccgacggcctcaaggccctggtggacgacctggccggactgcagttcgagctggaggccattaacacccggcgaggcagcggcaacggcggggcaggtggcagcgcgcacggccccggcggcggcgggcagcagggccaggggcaaggggcgccggatgaggtgcggctggcacagctgcgcggtgtggccgcgtcactgcagaagcgcgtggagtcggcgcgggcgcagcacgccgagctgcaggcgttgctgcacgtgtcggggcggctggtgagcaaggacgtgactgagaagctggacaagctgcacggcctgcacaaggagatggcgggctgggcgctggacggcggcttcatcgcgggacacgccgggctgctgctgtcggacctgagggcgctgcgtgaggtggtccaggagcacagccgggcgcacggcgaccagctggagggggacggcgagcacgggcacgaggcggtggacacgaacggccggcggtccctattcggctgggtgggcggcaagagcgagcgggccgacggggacggcgacggcccgcgccagctcgaccccgagtcggaggaggaggaggaggcgcgcgcagcaggcaaggacccgccgaaggcgatcaaggtgtaa

SEQ ID NO: 58 - protein, 732 aa, Chlamydomonas reinhardtii
MALTNAIIAATRAQEQNFDEDSGRMREIGAAREARGEGLAKGPGGGGGYGSSGGGFGIVHARTASEVASTGPSQWALLQERITAAQGSVPDPASDDVARLMRSIFMQHLMSGAPEYSKYFKNDIRMMQQEAELQKQAAKEAEASASGHRRMSTAGGSAGGASDAAGSPYSASAGRTASQPQLRPDHHHNDPPPNIFASLYRPCKHALARYRASPLRAKIYLTLSHPEYNAVAFTFGIFVMLVILLNTAVFCIESVPRWENTPLYDRLVIVDYVCLGIFTVEFVARLVTCSSLTHFWLNAMNWIDFFAIAPFYLELMIVGPDAGNQAASQTRIIRVLRLLRVLRLMRASTRFRNLQVVVDALVASGDVLGMLVFLLLVLLVVSATIIYFVEQALVEGSWFDSIPLTIYYMHVTLTTTGYGDFYPVSAWGRFIAGVFMLLCMVTLSLPISVIGGNFSNMWGRYTHIRDGIERSGVAWSNFIKLRGTATKHCAAMDDLIDIINRVKCALEDGTRGGGAVGQPGADGLKALVDDLAGLQFELEAINTRRGSGNGGAGGSAHGPGGGGQQGQGQGAPDEVRLAQLRGVAASLQKRVESARAQHAELQALLHVSGRLVSKDVTEKLDKLHGLHKEMAGWALDGGFIAGHAGLLLSDLRALREVVQEHSRAHGDQLEGDGEHGHEAVDTNGRRSLFGWVGGKSERADGDGDGPRQLDPESEEEEEARAAGKDPPKAIKV

SEQ ID NO: 59 - DNA, 2196 bp, Artificial Sequence
  Codon optimized sequence
atggctctgactaacgctattattgcggctacgcgcgctcaggagcagaacttcgacgaggactcgggccggatgcgcgagatcggggctgcgcgggaggctcgcggggagggcctggcgaagggtccgggtggtgggggcggttacggcagcagcggcggtggcttcgggatcgtgcacgcgcgcaccgcctccgaggtggcctccactggcccttcccagtgggccctgctccaggagcgcatcaccgcggctcagggctccgtgcccgatcccgcgagcgacgatgtcgctcgcctgatgcggtcgattttcatgcaacacctgatgtccggcgctcccgagtattccaagtatttcaagaacgacatccggatgatgcagcaggaggccgagctccagaagcaggccgctaaggaggccgaggcgagcgcgagcggccatcggcggatgagcacggctggcggctccgctggcggcgcttccgatgccgccggttccccctactccgccagcgctggccgcaccgccagccaaccccagctgcgcccggaccaccaccataacgatcccccgcctaacatttttgcctccctgtaccggccttgcaagcatgccctcgcgcgctaccgcgcttcgcctctgcgcgcgaagatttacctcaccctgagccacccggagtataacgcggtggcgttcactttcggcatttttgtcatgctggtgatcctgctgaacaccgccgtcttttgcatcgagagcgtccctcggtgggagaacaccccgctgtatgaccgcctggtgatcgtggattacgtctgcctcggcatcttcactgtggagtttgtcgcccggctggtcacctgctcctccctgacccacttttggctgaacgccatgaactggattgatttctttgcgattgcgcctttctatctggagctgatgattgtgggccccgacgccggcaaccaagcggcgtcgcagactcggatcattcgcgtgctccgcctcctgcgggtgctccggctcatgcgcgcgtcgacccgcttccggaacctgcaagtggtggtggatgcgctggtggcgtcgggcgatgtcctggggatgctggtgttcctcctgctggtgctgctggtggtcagcgcgacgatcatttatttcgtggagcaagccctggtcgagggttcctggttcgactcgattccgctcaccatctactatatgcacgtcaccctgacgaccacgggctacggcgacttctaccctgtctccgcctgggggcggttcatcgccggcgtgtttatgctgctgtgcatggtcaccctgtcgctgcccatcagcgtgattgggggcaacttcagcaacatgtggggccggtacacgcacatccgcgacggcattgagcggtcgggtgtggcctggtccaacttcatcaagctccgcgggacggcgaccaagcactgcgccgcgatggacgacctgatcgacatcattaaccgcgtgaagtgcgccctggaggacgggactcgcgggggtggcgccgtcggtcagcccggtgcggacgggctcaaggccctggtggatgacctcgccggcctccagtttgagctggaggcgatcaacactcgccgggggtccggtaacgggggtgcgggtgggtcggcgcatgggcctgggggcggtggtcaacaggggcaaggccagggtgcccccgacgaggtgcgcctcgcccaactgcggggcgtggcggcgtcgctgcaaaagcgggtggagtccgctcgcgcccagcacgccgagctccaggccctgctgcacgtgtcgggtcgcctggtgtcgaaggatgtgacggagaagctcgataagctgcacggcctgcataaggagatggccgggtgggcgctggatgggggctttatcgcgggtcacgcgggcctcctgctcagcgacctgcgcgctctgcgggaggtggtgcaggagcacagccgcgctcatggcgaccagctggagggggacggcgagcacggtcatgaggctgtggatacgaacggtcggcgctcgctgtttggctgggtcggcggcaagtcggagcgggccgatggcgacggcgacgggcctcgccagctcgatcctgagtcggaggaggaggaggaggctcgcgctgcgggtaaggaccctccgaaggctatcaaggtg

SEQ ID NO: 60 - DNA, 2292 bp, Artificial Sequence
  Codon optimized sequence
atgctcgagatggctctgactaacgctattattgcggctacgcgcgctcaggagcagaacttcgacgaggactcgggccggatgcgcgagatcggggctgcgcgggaggctcgcggggagggcctggcgaagggtccgggtggtgggggcggttacggcagcagcggcggtggcttcgggatcgtgcacgcgcgcaccgcctccgaggtggcctccactggcccttcccagtgggccctgctccaggagcgcatcaccgcggctcagggctccgtgcccgatcccgcgagcgacgatgtcgctcgcctgatgcggtcgattttcatgcaacacctgatgtccggcgctcccgagtattccaagtatttcaagaacgacatccggatgatgcagcaggaggccgagctccagaagcaggccgctaaggaggccgaggcgagcgcgagcggccatcggcggatgagcacggctggcggctccgctggcggcgcttccgatgccgccggttccccctactccgccagcgctggccgcaccgccagccaaccccagctgcgcccggaccaccaccataacgatcccccgcctaacatttttgcctccctgtaccggccttgcaagcatgccctcgcgcgctaccgcgcttcgcctctgcgcgcgaagatttacctcaccctgagccacccggagtataacgcggtggcgttcactttcggcatttttgtcatgctggtgatcctgctgaacaccgccgtcttttgcatcgagagcgtccctcggtgggagaacaccccgctgtatgaccgcctggtgatcgtggattacgtctgcctcggcatcttcactgtggagtttgtcgcccggctggtcacctgctcctccctgacccacttttggctgaacgccatgaactggattgatttctttgcgattgcgcctttctatctggagctgatgattgtgggccccgacgccggcaaccaagcggcgtcgcagactcggatcattcgcgtgctccgcctcctgcgggtgctccggctcatgcgcgcgtcgacccgcttccggaacctgcaagtggtggtggatgcgctggtggcgtcgggcgatgtcctggggatgctggtgttcctcctgctggtgctgctggtggtcagcgcgacgatcatttatttcgtggagcaagccctggtcgagggttcctggttcgactcgattccgctcaccatctactatatgcacgtcaccctgacgaccacgggctacggcgacttctaccctgtctccgcctgggggcggttcatcgccggcgtgtttatgctgctgtgcatggtcaccctgtcgctgcccatcagcgtgattgggggcaacttcagcaacatgtggggccggtacacgcacatccgcgacggcattgagcggtcgggtgtggcctggtccaacttcatcaagctccgcgggacggcgaccaagcactgcgccgcgatggacgacctgatcgacatcattaaccgcgtgaagtgcgccctggaggacgggactcgcgggggtggcgccgtcggtcagcccggtgcggacgggctcaaggccctggtggatgacctcgccggcctccagtttgagctggaggcgatcaacactcgccgggggtccggtaacgggggtgcgggtgggtcggcgcatgggcctgggggcggtggtcaacaggggcaaggccagggtgcccccgacgaggtgcgcctcgcccaactgcggggcgtggcggcgtcgctgcaaaagcgggtggagtccgctcgcgcccagcacgccgagctccaggccctgctgcacgtgtcgggtcgcctggtgtcgaaggatgtgacggagaagctcgataagctgcacggcctgcataaggagatggccgggtgggcgctggatgggggctttatcgcgggtcacgcgggcctcctgctcagcgacctgcgcgctctgcgggaggtggtgcaggagcacagccgcgctcatggcgaccagctggagggggacggcgagcacggtcatgaggctgtggatacgaacggtcggcgctcgctgtttggctgggtcggcggcaagtcggagcgggccgatggcgacggcgacgggcctcgccagctcgatcctgagtcggaggaggaggaggaggctcgcgctgcgggtaaggaccctccgaaggctatcaaggtgcccggggattacaaggatgacgacgacaagtccggcgagaacctgtacttccagggccacaaccatcgccataagcacaccggttga

SEQ ID NO: 61 - protein, 763 aa, Chlamydomonas reinhardtii
  MISC_FEATURE (1)..(3): Tag
MLEMALTNAIIAATRAQEQNFDEDSGRMREIGAAREARGEGLAKGPGGGGGYGSSGGGFGIVHARTASEVASTGPSQWALLQERITAAQGSVPDPASDDVARLMRSIFMQHLMSGAPEYSKYFKNDIRMMQQEAELQKQAAKEAEASASGHRRMSTAGGSAGGASDAAGSPYSASAGRTASQPQLRPDHHHNDPPPNIFASLYRPCKHALARYRASPLRAKIYLTLSHPEYNAVAFTFGIFVMLVILLNTAVFCIESVPRWENTPLYDRLVIVDYVCLGIFTVEFVARLVTCSSLTHFWLNAMNWIDFFAIAPFYLELMIVGPDAGNQAASQTRIIRVLRLLRVLRLMRASTRFRNLQVVVDALVASGDVLGMLVFLLLVLLVVSATIIYFVEQALVEGSWFDSIPLTIYYMHVTLTTTGYGDFYPVSAWGRFIAGVFMLLCMVTLSLPISVIGGNFSNMWGRYTHIRDGIERSGVAWSNFIKLRGTATKHCAAMDDLIDIINRVKCALEDGTRGGGAVGQPGADGLKALVDDLAGLQFELEAINTRRGSGNGGAGGSAHGPGGGGQQGQGQGAPDEVRLAQLRGVAASLQKRVESARAQHAELQALLHVSGRLVSKDVTEKLDKLHGLHKEMAGWALDGGFIAGHAGLLLSDLRALREVVQEHSRAHGDQLEGDGEHGHEAVDTNGRRSLFGWVGGKSERADGDGDGPRQLDPESEEEEEARAAGKDPPKAIKVPGDYKDDDDKSGENLYFQGHNHRHKHTG

SEQ ID NO: 62 - protein, 28 aa, Artificial Sequence
  FLAG-TEV-MAT tag
PGDYKDDDDKSGENLYFQGHNHRHKHTG